(19) United States
(12) Patent Application Publication
(10) Pub. No.: US 2023/0241071 A1
(43) Pub. Date: Aug. 3, 2023

(54) COMBINATION TREATMENT OF LIVER DISORDERS

(71) Applicant: Terns Pharmaceuticals, Inc., Foster City, CA (US)

(72) Inventors: Martijn FENAUX, San Mateo, CA (US); Kevin KLUCHER, Foster City, CA (US); Christopher T. JONES, Foster City, CA (US); Thorsten A. KIRSCHBERG, San Carlos, CA (US); Yujin WANG, San Jose, CA (US)

(21) Appl. No.: 17/985,738

(22) Filed: Nov. 11, 2022

Related U.S. Application Data
(60) Provisional application No. 63/263,933, filed on Nov. 11, 2021.
(60) Provisional application No. 63/349,977, filed on Jun. 7, 2022.

(51) Int. Cl. A61K 31/53; A61K 9/00; A61P 1/16; A61K 31/506

(52) U.S. Cl. CPC A61K 31/53; A61K 9/0053; A61P 1/16; A61K 31/506

(57) ABSTRACT

Provided herein are methods for treating liver disorders, including non-alcoholic steatohepatitis (NASH), and symptoms and manifestations thereof, in a patient which utilize, among others, a combination treatment of an SSAO inhibitor and a THR-β agonist. Also provided are fixed-dose combinations of an SSAO inhibitor and a THR-β agonist for use in treating NASH.

### CROSS REFERENCE TO RELATED APPLICATIONS

This application claims the benefit of and priority to U.S. Provisional Application Nos. 63/263,933 filed on Nov. 11, 2021, and 63/349,977 filed on Jun. 7, 2022. The contents of the aforementioned patent applications are incorporated herein by reference in their entirety for all purposes.

### FIELD OF THE INVENTION

This invention relates to methods and compositions for treating liver disorder in a patient.

### BACKGROUND

Fatty liver disease (FLD) encompasses a spectrum of disease states characterized by excessive accumulation of fat in the liver often accompanied with inflammation. FLD can lead to non-alcoholic fatty liver disease (NAFLD), which may be characterized by insulin resistance. If untreated, NAFLD can progress to a persistent inflammatory response or non-alcoholic steatohepatitis (NASH), progressive liver fibrosis, and eventually to cirrhosis. In Europe and the US, NAFLD is the second most common reason for liver transplantation. Accordingly, the need for treatment is urgent, but due to the lack of obvious symptoms to the patient, patients may lack the motivation to maintain treatment regimens, particularly burdensome treatment regimens, such as injected medicines, medications that are administered many times a day, or any that produce dangerous or irritating side effects. There is currently no approved treatment of NASH.

### BRIEF SUMMARY

Provided herein are methods and compositions for treating a liver disorder such as NASH in a patient in need thereof. The methods comprise administering to the patient a Semicarbazide-Sensitive Amine Oxidase (SSAO)/Vascular Adhesion Protein-1 (VAP-1) inhibitor and a thyroid hormone receptor beta (THR-β) agonist, as described herein. In some embodiments, the methods comprise administering to the patient a Semicarbazide-Sensitive Amine Oxidase (SSAO)/Vascular Adhesion Protein-1 (VAP-1) inhibitor, a thyroid hormone receptor beta (THR-β) agonist, and a Farnesoid X Receptor (FXR) agonist, as described herein.

In one aspect, the disclosure provides methods of reducing hepatic inflammation in a patient in need thereof, comprising administering to the patient a therapeutically effective amount of an SSAO inhibitor and a therapeutically effective amount of a THR-β agonist. In some embodiments, the disclosure provides methods of reducing hepatic inflammation in a patient in need thereof, comprising administering to the patient a therapeutically effective amount of an SSAO inhibitor, a therapeutically effective amount of a THR-β agonist, and a FXR agonist, as described herein.

In another aspect, the disclosure provides methods of treating a disease or condition characterized by fibrosis of the liver, comprising administering to the patient a therapeutically effective amount of an SSAO inhibitor and a therapeutically effective amount of a THR-β agonist. In some embodiments, the disclosure provides methods of treating a disease or condition characterized by fibrosis of the liver, comprising administering to the patient a therapeutically effective amount of an SSAO inhibitor, a therapeutically effective amount of a THR-β agonist, and a FXR agonist, as described herein.

In another aspect, the disclosure provides methods of treating a disease or condition characterized by hepatic steatosis, comprising administering to the patient a therapeutically effective amount of an SSAO inhibitor and a therapeutically effective amount of a THR-β agonist. In some embodiments, the disclosure provides methods of treating a disease or condition characterized by hepatic steatosis, comprising administering to the patient a therapeutically effective amount of an SSAO inhibitor, a therapeutically effective amount of a THR-β agonist, and a FXR agonist, as described herein.

In another aspect, the disclosure provide methods of treating or preventing NASH in a patient in need thereof, said method comprising administering to the patient a therapeutically effective amount of an SSAO inhibitor and a therapeutically effective amount of a THR-β agonist. In some embodiments, the disclosure provides methods of treating or preventing NASH in a patient in need thereof, said method comprising administering to the patient a therapeutically effective amount of an SSAO inhibitor, a therapeutically effective amount of a THR-β agonist, and a FXR agonist, as described herein. In one embodiment, the patient in need thereof is a patient that suffers from fatty liver disease such as NAFLD. In another embodiment, the patient in need thereof is a patient that suffers from metabolic syndrome.

In some embodiments, the SSAO inhibitor and the THR-β agonist, and optionally the FXR agonist, are administered simultaneously. In some such embodiments, the SSAO inhibitor and the THR-β agonist, and optionally the FXR agonist, are provided as a fixed-dose composition in a single pharmaceutical composition as set forth herein. In other embodiments, the SSAO inhibitor and the THR-β agonist, and optionally the FXR agonist, are administered sequentially. In some embodiments, either or both of the SSAO inhibitor and the THR-β agonist are administered orally. In some embodiments, at least one of the SSAO inhibitor and the THR-β agonist and the FXR agonist are administered orally.

In some embodiments, the patient has a liver disorder and diabetes mellitus. In some embodiments, the patient has a liver disorder and a cardiovascular disorder. In some embodiments, the treatment period is the remaining lifespan of the patient. In some embodiments, the method does not comprise administering an antihistamine, an immunosuppressant, a steroid, rifampicin, an opioid antagonist, or a selective serotonin reuptake inhibitor (SSRI).

In some embodiments, the SSAO inhibitor is administered once daily. In some embodiments, the SSAO inhibitor is administered twice daily. In some embodiments, the THR-β agonist is administered once daily. In some embodiments, the THR-β agonist is administered twice daily. In some embodiments, the FXR agonist is administered once daily. In some embodiments, the FXR agonist is administered twice daily. In some embodiments, the administration comprises administering the SSAO inhibitor daily for a treatment period of one or more weeks. In some embodiments, the administration comprises administering the THR-β agonist daily for a treatment period of one or more weeks. In some embodiments, the administration comprises administering the FXR agonist daily for a treatment period of one or more weeks. In some embodiments, the administration comprises administering the SSAO inhibitor daily and the THR-β agonist daily for a treatment period of one or more weeks. In some embodiments, the administration comprises administering the SSAO inhibitor daily, the THR-β agonist daily, and the FXR agonist daily for a treatment period of one or more weeks.

A variety of different SSAO inhibitors and THR-β agonists, and optionally FXR agonists, can be used to achieve the beneficial effects observed on liver disease as discussed herein. For instance, in some embodiments, the SSAO inhibitor administered to the patient in need thereof is a compound of Formula (I)

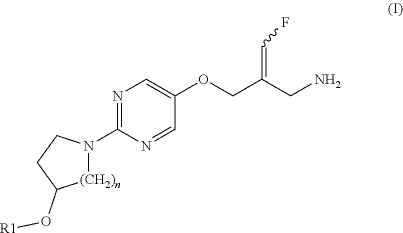

wherein:
n is 1 or 2; and

### R1 is H or —CH₃,

or a pharmaceutically acceptable salt thereof.

In some embodiments, the SSAO inhibitor administered to the patient in need thereof is a compound of Formula (I), where n is 1, or a pharmaceutically acceptable salt thereof. In another embodiment, the SSAO inhibitor is a compound of Formula (I), where n is 2, or a pharmaceutically acceptable salt thereof.

In some embodiments, the SSAO inhibitor administered to the patient in need thereof is a compound of Formula (I), where R1 is H, or a pharmaceutically acceptable salt thereof. In yet another embodiment, the present invention provides a compound of Formula (II), where R1 is —CH₃, or a pharmaceutically acceptable salt thereof.

In some embodiments, the SSAO inhibitor administered to the patient in need thereof is

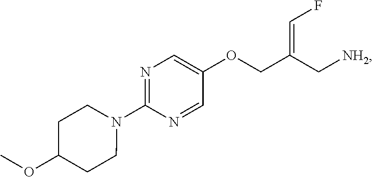 [SMILES: COC1CCN(c2ncc(OC/C(=C/F)CN)cn2)CC1]

which is (E)-3-fluoro-2-(((2-(4-methoxypiperidin-1-yl)pyrimidin-5-yl)oxy)methyl)prop-2-en-1-amine, or a pharmaceutically acceptable salt thereof. In some embodiments, the SSAO inhibitor administered to the patient in need thereof is (E)-3-fluoro-2-(((2-(4-methoxypiperidin-1-yl)pyrimidin-5-yl)oxy)methyl)prop-2-en-1-amine dihydrochloride. In some embodiments, the SSAO inhibitor administered to the patient in need thereof is (E)-3-fluoro-2-(((2-(4-methoxypiperidin-1-yl)pyrimidin-5-yl)oxy)methyl)prop-2-en-1-amine 4-methylbenzenesulfonate.

In some embodiments, the THR-β agonist administered to the patient in need thereof is resmetirom (MGL-3196). In some embodiments, the THR-β agonist is administered to the patient in need thereof VK2809 (by Viking Therapeutics). In some embodiments, the THR-β agonist administered to the patient in need thereof is sobetirome. In some embodiments, the THR-β agonist administered to the patient in need thereof is eprotirome. In some embodiments, the THR-β agonist administered to the patient in need thereof is ALG-055009 (by Aligo). In some embodiments, the THR-β agonist administered to the patient in need thereof is CNPT-101101. In some embodiments, the THR-β agonist administered to the patient in need thereof is CNPT-101207. In some embodiments, the THR-β agonist administered to the patient in need thereof is ASC41 (by Ascletis).

In some embodiments, the THR-β agonist is a compound of Formula (II)

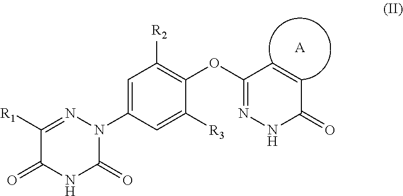

wherein:
R₁ is selected from the group consisting of hydrogen, cyano, substituted or unsubstituted C₁₋₆ alkyl, and substituted or unsubstituted C₃₋₆ cycloalkyl, the substituent being selected from the group consisting of halogen atoms, hydroxy, and C₁₋₆ alkoxy;
R₂ and R₃ are each independently selected from the group consisting of halogen atoms and substituted or unsubstituted C₁₋₆ alkyl, the substituent being selected from the group consisting of halogen atoms, hydroxy, and C₁₋₆ alkoxy;
ring A is a substituted or unsubstituted saturated or unsaturated C₅₋₁₀ aliphatic ring, or a substituted or unsubstituted C₅₋₁₀ aromatic ring, the substituent being one or more substances selected from the group consisting of hydrogen, halogen atoms, hydroxy, —OCF₃, —NH₂, —NHC₁₋₄ alkyl, —N(C₁₋₄ alkyl)₂, —CONH₂, —CONHC₁₋₄ alkyl, —CON(C₁₋₄ alkyl)₂, —NHCOC₁₋₄ alkyl, C₁₋₆ alkyl, C₁₋₆ alkoxy or C₃₋₆ cycloalkyl, and when two substituents are contained, the two substituents can form a ring structure together with the carbon connected thereto; and the halogen atoms are selected from the group consisting of F, Cl and Br,
or a pharmaceutically acceptable salt thereof.

In some embodiments, the THR-β agonist administered to the patient in need thereof is a compound of Formula (IIa)

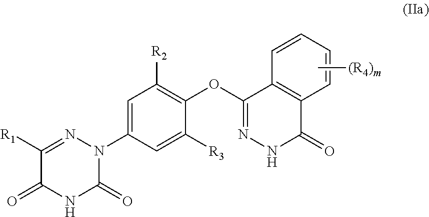

wherein:
R₁ to R₃ are defined as detailed herein for Formula (II);
R₄ is selected from the group consisting of hydrogen, halogen atoms, hydroxy, —OCF₃, —NH₂, —NHC₁₋₄ alkyl, —N(C₁₋₄ alkyl)₂, —CONH₂, —CONHC₁₋₄ alkyl, —CON(C₁₋₄ alkyl)₂, —NHCOC₁₋₄ alkyl, C₁₋₆ alkyl, C₁₋₆ alkoxy and C₃₋₆ cycloalkyl;
m is an integer from the range 1 to 4; and
the halogen atoms are selected from the group consisting of F, Cl and Br.
or a pharmaceutically acceptable salt thereof.

In some embodiments, wherein R₄ is selected from the group consisting of hydrogen, halogen atoms, hydroxy, —OCF₃, C₁₋₆ alkyl, C₁₋₆ alkoxy and C₃₋₆ cycloalkyl; and m is an integer from the range 1 to 3.

In some embodiments, wherein R₁ is selected from the group consisting of hydrogen, cyano, and substituted or unsubstituted C₁₋₆ alkyl, the substituent being selected from the group consisting of halogen atoms, hydroxy, and C₁₋₆ alkoxy; and the halogen atoms are selected from the group consisting of F, Cl and Br.

In some embodiments, the THR-β agonist is

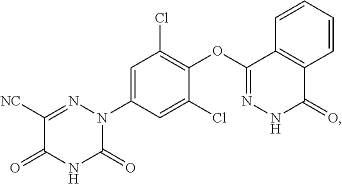 [SMILES: N#Cc1nn(-c2cc(Cl)c(Oc3n[nH]c(=O)c4ccccc34)c(Cl)c2)c(=O)[nH]c1=O]

which is 2-(3,5-dichloro-4-((4-oxo-3,4-dihydrophthalazin-1-yl)oxy)phenyl)-3,5-dioxo-2,3,4,5-tetrahydro-1,2,4-triazine-6-carbonitrile, or a pharmaceutically acceptable salt thereof. In some embodiments, the THR-β agonist is 2-(3,5-dichloro-4-((4-oxo-3,4-dihydrophthalazin-1-yl)oxy)phenyl)-3,5-dioxo-2,3,4,5-tetrahydro-1,2,4-triazine-6-carbonitrile potassium salt. In some embodiments, the THR-β agonist is 2-(3,5-dichloro-4-((4-oxo-3,4-dihydrophthalazin-1-yl)oxy)phenyl)-3,5-dioxo-2,3,4,5-tetrahydro-1,2,4-triazine-6-carbonitrile sodium salt.

In some embodiments, the FXR agonist is obeticholic acid. In some embodiments, the FXR agonist is cilofexor. In some embodiments, the FXR agonist is tropifexor. In some embodiments, the FXR agonist is EYP001 (Vonafexor, proposed INN). In some embodiments, the FXR agonist is MET409 (Metacrine). In some embodiments, the FXR agonist is EDP-305 (by Enanta).

In some embodiments, the FXR agonist is a compound of formula (III):

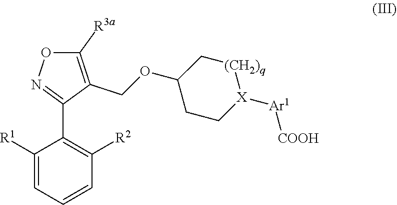

wherein:
q is 1 or 2;
R¹ is chloro, fluoro, or trifluoromethoxy;
R² is hydrogen, chloro, fluoro, or trifluoromethoxy;
R^{3a} is trifluoromethyl, cyclopropyl, or isopropyl;

### X is CH or N,

provided that when X is CH, q is 1; and
Ar¹ is indolyl, benzothienyl, naphthyl, phenyl, benzoisothiazolyl, indazolyl, or pyridinyl, each of which is optionally substituted with methyl or phenyl,
or a pharmaceutically acceptable salt thereof.

In some embodiments, the FXR agonist is

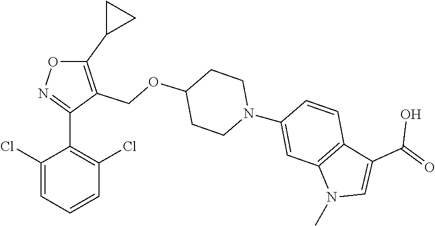 [SMILES: Cn1cc(C(=O)O)c2ccc(N3CCC(OCc4c(-c5c(Cl)cccc5Cl)noc4C4CC4)CC3)cc21]

or a pharmaceutically acceptable salt thereof.

In some embodiments, provided are methods of treating a liver disorder in a patient in need thereof with a Semicarbazide-Sensitive Amine Oxidase (SSAO) inhibitor and a thyroid hormone receptor beta (THR-β) agonist, and optionally an FXR agonist, comprising administering a therapeutically effective amount of the SSAO inhibitor, wherein the SSAO inhibitor is

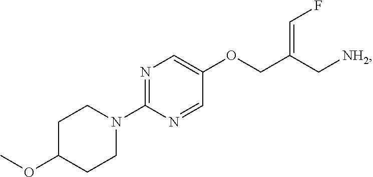 [SMILES: COC1CCN(c2ncc(OC/C(=C/F)CN)cn2)CC1]

or a pharmaceutically acceptable salt thereof, and administering a therapeutically effective amount of the THR-β agonist, wherein the THR-β agonist is

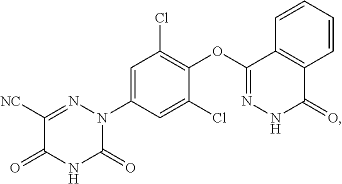 [SMILES: N#Cc1nn(-c2cc(Cl)c(Oc3n[nH]c(=O)c4ccccc34)c(Cl)c2)c(=O)[nH]c1=O]

or a pharmaceutically acceptable salt thereof, and optionally administering a therapeutically effective amount of the FXR agonist, wherein the liver disorder is selected from liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH). In some embodiments, the liver disorder is NASH.

In some embodiments, provided are methods of treating a liver disorder in a patient in need thereof with a Semicarbazide-Sensitive Amine Oxidase (SSAO) inhibitor and a thyroid hormone receptor beta (THR-β) agonist, comprising administering a therapeutically effective amount of the SSAO inhibitor, wherein the SSAO inhibitor is

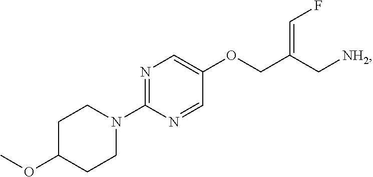 [SMILES: COC1CCN(c2ncc(OC/C(=C/F)CN)cn2)CC1]

or a pharmaceutically acceptable salt thereof, and administering a therapeutically effective amount of the THR-β agonist, wherein the THR-β agonist is

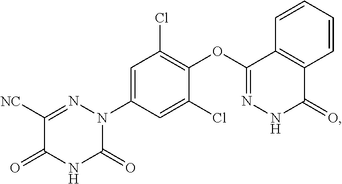 [SMILES: N#Cc1nn(-c2cc(Cl)c(Oc3n[nH]c(=O)c4ccccc34)c(Cl)c2)c(=O)[nH]c1=O]

or a pharmaceutically acceptable salt thereof, and optionally administering a therapeutically effective amount of the FXR agonist, wherein the FXR agonist is

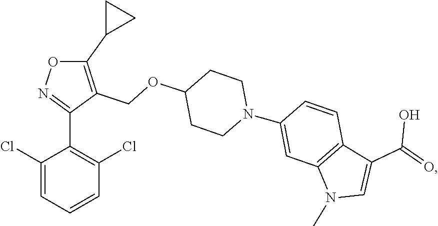 [SMILES: Cn1cc(C(=O)O)c2ccc(N3CCC(OCc4c(-c5c(Cl)cccc5Cl)noc4C4CC4)CC3)cc21]

or a pharmaceutically acceptable salt thereof, wherein the liver disorder is selected from liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH). In some embodiments, the liver disorder is NASH.

In some embodiments, provided are methods of treating a liver disorder in a patient in need thereof with a Semicarbazide-Sensitive Amine Oxidase (SSAO) inhibitor and a thyroid hormone receptor beta (THR-β) agonist, comprising administering a therapeutically effective amount of the SSAO inhibitor, wherein the SSAO inhibitor is

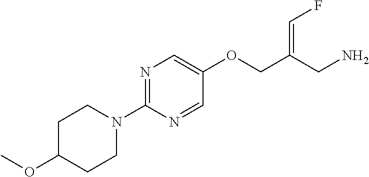 [SMILES: COC1CCN(c2ncc(OC/C(=C/F)CN)cn2)CC1]

4-methylbenzene sulfonate salt, and administering a therapeutically effective amount of the THR-β agonist, wherein the THR-β agonist is

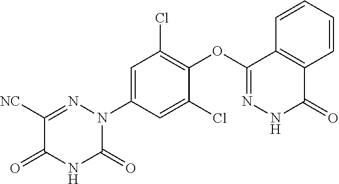 [SMILES: N#Cc1nn(-c2cc(Cl)c(Oc3n[nH]c(=O)c4ccccc34)c(Cl)c2)c(=O)[nH]c1=O]

potassium salt, and optionally administering a therapeutically effective amount of the FXR agonist, wherein the FXR agonist is

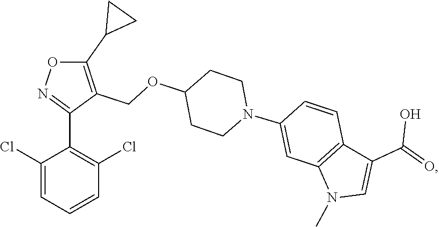 [SMILES: Cn1cc(C(=O)O)c2ccc(N3CCC(OCc4c(-c5c(Cl)cccc5Cl)noc4C4CC4)CC3)cc21]

wherein the liver disorder is selected from liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH). In some embodiments, the liver disorder is NASH.

### BRIEF DESCRIPTION OF THE FIGURES

FIG. 1A shows the plasma SSAO-specific amine oxidase activity compared to baseline of healthy volunteers administered a single dose of placebo or 1, 3, 6, or 10 mg of Compound 1 at 4 hours and 168 hours post dose. FIG. 1B shows a time course of plasma total amine oxidase activity compared to baseline of healthy volunteers administered a single dose of placebo or 1, 3, 6, or 10 mg of Compound 1. FIG. 1C shows a time course of the level of Compound 1 after a single dose of placebo or 1, 3, 6, or 10 mg in healthy volunteers. FIG. 1D shows a time course of plasma methylamine concentration after a single dose of placebo or 1, 3, 6, or 10 mg of Compound 1 in healthy volunteers.

FIG. 2A shows a time course of the concentration of Compound 1 after a single dose of 1, 4, or 10 mg of Compound 1 in healthy volunteers. FIG. 2B shows a time course of the concentration of Compound 1 on the last day of 7 daily doses of 1 mg or 4 mg of Compound 1, and the last day of 14 daily doses of 10 mg of Compound 1.

FIG. 3A shows SSAO-specific amine oxidase activity 24 hours after a first daily dose of 1, 4, or 10 mg Compound 1 in healthy volunteers. FIG. 3B shows a time course of plasma methylamine concentration after a first daily dose of 1, 4, or 10 mg Compound 1 in healthy volunteers. FIG. 3C shows a time course of plasma methylamine concentration after the last day of 7 daily doses of 1 mg or 4 mg of Compound 1, and the last day of 14 daily doses of 10 mg of Compound 1, both in healthy volunteers. FIG. 3D shows a time course of total amine oxidase activity (percent change from baseline) after a single dose of 1, 4, or 10 mg of Compound 1 in healthy volunteers. FIG. 3E shows a 14 day time course of total amine oxidase activity (percent change from baseline) after the 6^{th} of 7 daily doses of 1 mg or 4 mg of Compound 1, or the 13^{th} of 14 daily doses of 10 mg of Compound 1, in healthy volunteers.

FIG. 4A shows the effect of Compound 2 on serum cholesterol in rat hypercholesterolemic model. FIG. 4B shows the effect of Compound 2 on serum triglycerides in rat hypercholesterolemic model.

FIG. 5 shows the effects of Compound 2 on body and organ weight in mouse NASH model.

FIG. 6 shows the effects of Compound 2 on liver steatosis, inflammation, and fibrosis in mouse NASH model.

FIG. 7 shows the effects of Compound 2 on lipids and indicators of liver injury (ALT) in mouse NASH model.

FIG. 8 shows the effects of Compound 2 on expression of genes associated with collagen extracellular matrix and hepatic stellate cell activation.

FIG. 9 shows the plasma concentration of Compound 2 in beagle dogs after administration of a 50 mg capsule of Compound 2 with or without 5 wt % SLS.

FIG. 10 compares the plasma concentration of Compound 2 in beagle dogs after pre-treatment with pentagastrin or famotidine.

FIG. 11 compares the plasma concentration of Compound 2 in beagle dogs under a fasted or fed state.

FIG. 12 shows the heart rate of healthy human patients upon administration of a single dose of placebo or compound 2 at 3 mg, 10 mg, 30 mg, and 60 mg doses.

FIG. 13A shows the plasma concentration of Compound 2 in healthy humans after administration with compound 2 at 3 mg, 10 mg, 30 mg, and 60 mg doses. FIG. 13B correlates the AUC and Cmax to each dose.

FIG. 14A shows the percent change from baseline of sex hormone binding globulin (SHBG) after administration with a single dose of compound 2 at 3 mg, 10 mg, 30 mg, and 60 mg doses. FIG. 14B shows the percent change from baseline of Apolipoprotein B (Apo B) after administration with a single dose of compound 2 at 3 mg, 10 mg, 30 mg, and 60 mg doses.

FIGS. 15A, 15B, and 15C show free T3, T4, and TSH, respectively, on day 15 after 14 days of daily administration of compound 2 or placebo in humans.

FIGS. 16A, 16B, and 16C show percent change in free testosterone, total testosterone, and sex hormone binding globulin (SHBG) from baseline on day 15 after 14 days of daily administration of compound 2 or placebo in humans.

FIG. 17 shows plasma concentrations of Compound 2 over time on days 1 and 14 of a multiple ascending dose study wherein Compound 2 was dosed once daily.

FIG. 18 shows percent change in pharmacodynamics markers (sex hormone binding globulin, ApoB, total cholesterol, LDL-c, HDL-c, and triglycerides) from baseline on day 15 after 14 days of daily administration of compound 2 or placebo in humans.

FIG. 19 shows the levels of Treg and M2 macrophage liver infiltration determined by single-sample gene set enrichment analysis. The analysis was performed on liver RNA sequencing data of CDHFD rats administered NaNO₂ and treated with Compound 3, Compound 1, or the combination of Compound 3 and Compound 1 (*p-value<0.05; ***p-value<0.001).

FIG. 20 shows expression analysis by RNA sequencing for markers of Treg and M2 macrophages in the liver of CDHFD rats administered NaNO₂ and treated with Compound 3, Compound 1, or the combination of Compound 3 and Compound 1. Ikzf2, IKAROS Family Zinc Finger 2 (Treg marker); Foxp3, Forkhead Box P3 (Treg marker); Cd163 (M2 macrophage marker). (*p-value<0.05; **p-value<0.01.)

FIG. 21 shows the number and overlap of differentially expressed genes (DEGs) identified by RNA sequencing analysis in the liver of CDHFD rats administered NaNO₂ and treated with Compound 3, Compound 1, or the combination of Compound 3 and Compound 1, relative to a vehicle NASH control using fold-change and p-value cutoffs of ≥1.5 and 0.01, respectively.

FIG. 22 shows differential gene expression analysis of select biological processes in a mouse model of NASH treated with 3 mg/kg Compound 3 and/or 1 mg/kg Compound 2.

FIG. 23 shows the number and overlap of differentially expressed genes (DEGs) identified in a mouse model of NASH treated with 3 mg/kg Compound 3, 1 mg/kg Compound 2, or 3 mg/kg Compound 3 and 1 mg/kg Compound 2, relative to a vehicle NASH control.

FIG. 24 shows the number and overlap of biological processes that were significantly enriched in a mouse model of NASH treated with 3 mg/kg Compound 3, 1 mg/kg Compound 2, or 3 mg/kg Compound 3 and 1 mg/kg Compound 2, relative to a vehicle NASH control.

FIG. 25 shows liver steatosis, inflammation, and fibrosis, as well as serum triglyceride, total cholesterol, and alanine aminotransferase (ALT) in a mouse model of NASH treated with 3 mg/kg Compound 3, 1 mg/kg Compound 2, or 3 mg/kg Compound 3 and 1 mg/kg Compound 2, relative to a vehicle NASH control.

FIG. 26 shows expression levels of genes associated with FXR and THRβ pathways in a mouse model of NASH treated with 3 mg/kg Compound 3, 1 mg/kg Compound 2, or 3 mg/kg Compound 3 and 1 mg/kg Compound 2, relative to a vehicle NASH control.

FIG. 27 shows mean expression levels (count per million reads, CPM) of genes associated with fibrosis and inflammation pathways after treatment with the combination of Compound 3 and Compound 2, which were determined by RNAseq. *p<0.05, **p<0.01, ***p<0.001, ****p<0.0001 in a mouse model of NASH vs. vehicle (NASH) control.

FIG. 28 shows the study design for Compound 1.

FIG. 29 shows patient demographics and baseline characteristics.

FIG. 30 shows the patient disposition according to the study design.

FIG. 31 shows overall summary of adverse events.

FIG. 32A shows TIMP-1 change from baseline to week 6 and week 12.

FIG. 32B shows results regarding NASH and inflammation biomarkers mean (SE) change from baseline to week 12.

FIG. 33 shows cT1 changes in week 12.

FIG. 34 shows VAP-1/SSAO activity at week 12 relative to baseline, %.

FIG. 35 shows ELF components (TIMP-1, P3NP, HA) change from baseline to week 12.

FIG. 36 shows change in ICAM-1 and VCAM-1 from baseline to week 12.

FIG. 37 shows the overall study of compound 2 in healthy volunteers.

FIG. 38 shows the demographics and baseline characteristics for compound 2.

FIG. 39 shows the plasma concentration-time profile, day 14, for compound 2.

FIG. 40 shows the day 14, PK parameters of compound 2.

FIG. 41 shows the sex hormone binding globulin (SHBG) (percent change from baseline to Day 15).

FIG. 42 shows the LDL-c (percent change from baseline to Day 15).

FIG. 43 shows the percent change from baseline at end of treatment (Day 15) for SHBG and LDL-c per compound 2 dose.

FIG. 44 shows the decreases in total cholesterol (TC), Apo B, and triglycerides (TG) per compound 2 dose.

FIG. 45 shows that the treatment-emergent adverse events were mild and mostly unrelated with no significant changes in vital signs.

FIG. 46A and FIG. 46B show body weight change in male biopsy-confirmed DIO-NASH mice. Compound 3 alone and in combination with Compound 2 reduced body weight. Body weights were measured daily during study. Body weight change during study is shown in FIG. 46A, where dots represent mean body weight change relative to baseline (n=10-16 mice per group). In FIG. 46B, bars represent mean (SD) body weight measured on Week 11 of the study. Lean vehicle control, white; DIO-GAN vehicle control, gray; Compound 3, blue; Compound 2-low, light orange; Compound 2-med, orange; Compound 2-high, dark orange; Combo-low, light purple; Combo-med, purple; Combo-high, dark purple. Statistical comparison to DIO-GAN vehicle control determined by ANOVA followed by Tukey correction for multiple comparisons. *p<0.05; **p<0.01; ***p<0.001; ****p<0.0001.

FIG. 47 shows Discrete weekly food intake during the study. Dots represent mean weekly food intake in grams (n=10-16 mice per group). Lean vehicle control, white; DIO-GAN vehicle control, gray; Compound 3, blue; Compound 2-low, light orange; Compound 2-med, orange; Compound 2-high, dark orange; Combo-low, light purple; Combo-med, purple; Combo-high, dark purple.

FIG. 48A and FIG. 48B shows liver and spleen weight. Compound 3 and Compound 2 alone and in combination significantly reduced hepatomegaly without changes in spleen weight. Bars represent mean (SD) liver (FIG. 48A) and spleen (FIG. 48B) organ weights (n=10-16 mice per group) determined at the end of study. Statistical comparison to DIO-GAN vehicle control determined by ANOVA followed by Tukey correction for multiple comparisons. *p<0.05; **p<0.01; ***p<0.001; ****p<0.0001.

FIG. 49A and FIG. 49B show Body mass composition at baseline. Body mass composition was well balanced across treatment groups at baseline (Week −1). Bars represent mean (SD) fat tissue (FIG. 49A) and lean tissue (FIG. 49B) mass as a percentage of body weight (% BW, n=10-16 mice per group) determined by whole body EchoMRI at Week −1 of the study. Statistical comparison to DIO-GAN vehicle control determined by ANOVA followed by Tukey correction for multiple comparisons. *p<0.05; **p<0.01; ***p<0.001; ****p<0.0001.

FIG. 50A and FIG. 50B show body mass composition at week 11. Compound 3 alone and in combination with Compound 2 significantly reduced fat tissue mass. Bars represent mean (SD) fat tissue (FIG. 50A) and lean tissue (FIG. 50B) mass as a percentage of body weight (% BW, n=10-16 mice per group) determined by whole body EchoMRI at Week 11 of the study. Statistical comparison to DIO-GAN vehicle control determined by ANOVA followed by Tukey correction for multiple comparisons. *p<0.05; **p<0.01; ***p<0.001; ****p<0.0001.

FIG. 51A and FIG. 51B show plasma and liver total cholesterol. Compound 3 and Compound 2 alone and in combination significantly reduced total cholesterol. Bars represent mean (SD) total cholesterol levels measured at the end of study (n=10-16 mice per group) in plasma (FIG. 51A) and liver (FIG. 51B). Statistical comparison to DIO-GAN vehicle control determined by ANOVA followed by Tukey correction for multiple comparisons. *p<0.05; **p<0.01; ***p<0.001; ****p<0.0001.

FIG. 52A and FIG. 52B show plasma and liver triglycerides. Compound 3 alone and in combination with Compound 2 significantly reduced plasma triglycerides. Bars represent mean (SD) triglyceride levels measured at the end of study (n=10-16 mice per group) in plasma (FIG. 52A) and liver (FIG. 52B). Statistical comparison to DIO-GAN vehicle control determined by ANOVA followed by Tukey correction for multiple comparisons. *p<0.05; **p<0.01; ***p<0.001; ****p<0.0001.

FIG. 53A and FIG. 53B shows alanine aminotransferase (ALT) and aspartate aminotransferase (AST) levels. Compound 2 alone significantly reduced Alanine Aminotransferase (ALT) levels. Bars represent mean (SD) ALT (FIG. 53A) and Aspartate Aminotransferase (AST) (FIG. 53B) levels (n=10-16 mice per group) determined at the end of study. Statistical comparison to DIO-GAN vehicle control determined by ANOVA followed by Tukey correction for multiple comparisons. *p<0.05; **p<0.01; ***p<0.001; ****p<0.0001.

FIG. 54 shows alkaline phosphatase levels. Alkaline Phosphatase (ALP) levels were not significantly changed by treatment. Bars represent mean (SD) ALP levels (n=10-16 mice per group) determined at the end of study. Statistical comparison to DIO-GAN vehicle control determined by ANOVA followed by Tukey correction for multiple comparisons. *p<0.05; **p<0.01; ***p<0.001; ****p<0.0001.

FIG. 55A and FIG. 55B show NAFLD Activity Score (NAS) at Baseline and at End of Treatment. The NAFLD Activity Score (NAS) was well balanced at baseline and significantly improved in combination treatment groups. NAS, defined as the composite, unweighted sum of ballooning, steatosis, and lobular inflammation histological scores was determined at baseline (FIG. 55A) and after 12-weeks of treatment (FIG. 55B). Dots represent individual mice in each treatment group (n=14-16). Statistical comparison to DIO-GAN vehicle control determined by ANOVA followed by Tukey correction for multiple comparisons. *p<0.05, **p<0.01, ***p<0.001, ****p<0.0001.

FIG. 56A and FIG. 56B show liver steatosis by histological morphometric analysis. The combination of Compound 3 and Compound 2 resulted in greater reductions in liver steatosis as determined by histological morphometric analysis. Hepatocellular steatosis including the percentage of hepatocytes with lipid droplets and liver lipid content as a percent fractional area (FA) was determined by morphometric analysis of liver histological samples at the end of study. Statistical comparison to DIO-GAN vehicle control determined by ANOVA followed by Tukey correction for multiple comparisons. *p<0.05, **p<0.01, ***p<0.001, ****p<0.0001.

FIG. 57 shows hepatocyte lipid droplet size. Combination treatment significantly reduces hepatocyte lipid droplet size. Lipid droplet size was determined by morphometric analysis of liver histological samples at the end of study. Statistical comparison to DIO-GAN vehicle control determined by ANOVA followed by Tukey correction for multiple comparisons. *p<0.05, **p<0.01, ***p<0.001, ****p<0.0001.

FIG. 58 shows Plasma CK18 M30. Apoptosis biomarker cytokeratin 18 M30 (CK18 M30) levels were not significantly changed by treatment. CK18 M30, an apoptosis biomarker, was measured in plasma samples at the end of study. Statistical comparison to DIO-GAN vehicle control determined by ANOVA followed by Tukey correction for multiple comparisons. *p<0.05, **p<0.01, ***p<0.001, ****p<0.0001.

FIG. 59A and FIG. 59B show liver protein expression of Galectin-3 and smooth muscle actin proteins. Compound 3 treatment reduces expression of Galectin-3 (Gal-3). Expression of Gal-3 (FIG. 59A) and α-smooth muscle actin (α-SMA) (FIG. 59B) was assessed by immunohistochemical (IHC) staining of the livers of treated mice at the end of study. Statistical comparison to DIO-GAN vehicle control determined by ANOVA followed by Tukey correction for multiple comparisons. *p<0.05, **p<0.01, ***p<0.001, ****p<0.0001.

FIG. 60 shows expression of energy and lipid metabolic genes in the liver. Comparison of gene expression values between Compound 2-high and Combo-high treatment groups for select genes involved in energy and lipid metabolism. Liver samples were processed for transcriptomics analysis by RNAseq at termination. Dots represent mean fold-change (n=10 mice per group) expression values relative to DIO-GAN vehicle control. Red and blue coloring indicates the change direction (red=increased expression; blue=decreased expression) between the DIO-GAN vehicle and Lean control groups. Dotted line indicates line of identity. Solid line was generated by linear regression analysis of the fold-change expression values for select genes in the Compound 2-high and Combo-high treatment groups. Select genes involved in energy and lipid metabolism. Squalene epoxidase (Sqle), 7-dehydrocholesterol (Dhcr7), hydroxymethylgluteraryl-CoA synthase (Hmgcs1), and stearoyl-CoA desaturase (Scd1).

FIG. 61A, FIG. 61B, FIG. 61C, and FIG. 61D show expression of select genes involved in energy and lipid metabolism. Liver expression of genes involved in energy and lipid metabolism. Liver samples were processed for transcriptomics analysis by RNAseq at termination. Bars represent mean (SD) expression (FPKM) values for select genes involved in energy and lipid metabolism shown. Squalene epoxidase (Sqle, FIG. 61A), 7-dehydrocholesterol (Dhcr7, FIG. 61B), hydroxymethylgluteraryl-CoA synthase (Hmgcs1, FIG. 61C), and stearoyl-CoA desaturase (Scd1, FIG. 61D). Lean, white (left-most bar); DIO-GAN vehicle control, gray (second from left); Compound 3, blue (third from left); Compound 2-low, light orange (fourth from left); Compound 2-med, orange (fifth from left); Compound 2-high, dark orange (fourth from right); Combo-low, light purple (third from right); Combo-med, purple (second from right); Combo-high, dark purple (right-most bar). Statistical comparison for individual treatment group are shown relative to DIO-GAN vehicle control *p<0.05, **p<0.01, ***p<0.001, ****p<0.0001. Combination treatment groups were additionally compared to Compound 3 (+p<0.05, ++p<0.01, +++p<0.0001, ++++p<0.00001) or their respective Compound 2 single agent treatment groups (i.e., Compound 2-low vs Combo-low); #p<0.05, ##p<0.01, ###p<0.0001, ####p<0.00001.

FIG. 62 shows expression of genes involved in fibrosis and inflammation. Liver expression of genes involved in fibrosis and inflammation. Liver samples were processed for transcriptomics analysis by RNAseq at termination. Bars represent mean (SD) expression (FPKM) values for select genes involved in fibrosis and inflammation. Collagen type I alpha 1 (Col1a1), actin alpha 2 smooth actin (Acta2), Galectin 3 (Lgals3), and melanoma cell adhesion molecule (CD146). Lean, white (left-most bar); DIO-GAN vehicle control, gray (second from left); Compound 3, blue (third from left); Compound 2-low, light orange (fourth from left); Compound 2-med, orange (fifth from left); Compound 2-high, dark orange (fourth from right); Combo-low, light purple (third from right); Combo-med, purple (second from right); Combo-high, dark purple (right-most bar). Statistical comparison for individual treatment group are shown relative to DIO-GAN vehicle control *p<0.05, **p<0.01, ***p<0.001, ****p<0.0001.

### DETAILED DESCRIPTION

### Definitions

As used herein, the following definitions shall apply unless otherwise indicated. Further, if any term or symbol used herein is not defined as set forth below, it shall have its ordinary meaning in the art.

“Comprising” is intended to mean that the compositions and methods include the recited elements, but not exclude others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of, e.g., other ingredients and substantial method steps recited. Embodiments defined by each of these transition terms are within the scope of this invention.

“Combination therapy” or “combination treatment” refers to the use of two or more drugs or agents in treatment, e.g., the use of a compound of formula (I) or (II) as utilized herein together with another agent useful to treat liver disorders, such as NAFLD, NASH, and symptoms and manifestations of each thereof is a combination therapy. Administration in “combination” refers to the administration of two agents (e.g., a compound of formula (I) or (II) as utilized herein, and another agent) in any manner in which the pharmacological effects of both manifest in the patient at the same time. Thus, administration in combination does not require that a single pharmaceutical composition, the same dosage form, or even the same route of administration be used for administration of both agents or that the two agents be administered at precisely the same time. Both agents can also be formulated in a single pharmaceutically acceptable composition. A non-limiting example of such a single composition is an oral composition or an oral dosage form. For example, and without limitation, it is contemplated that a compound of formula (I) or (II) can be administered in combination therapy with another agent in accordance with the present invention.

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

“Patient” refers to mammals and includes humans and non-human mammals. Examples of patients include, but are not limited to mice, rats, hamsters, guinea pigs, pigs, rabbits, cats, dogs, goats, sheep, cows, and humans. In some embodiments, patient refers to a human.

“Pharmaceutically acceptable” refers to safe and non-toxic, preferably for in vivo, more preferably, for human administration.

“Pharmaceutically acceptable salt” refers to a salt that is pharmaceutically acceptable. A compound described herein may be administered as a pharmaceutically acceptable salt.

“Salt” refers to an ionic compound formed between an acid and a base. When the compound provided herein contains an acidic functionality, such salts include, without limitation, alkali metal, alkaline earth metal, and ammonium salts. As used herein, ammonium salts include, salts containing protonated nitrogen bases and alkylated nitrogen bases. Exemplary and non-limiting cations useful in pharmaceutically acceptable salts include Na, K, Rb, Cs, NH₄, Ca, Ba, imidazolium, and ammonium cations based on naturally occurring amino acids. When the compounds utilized herein contain basic functionality, such salts include, without limitation, salts of organic acids, such as carboxylic acids and sulfonic acids, and mineral acids, such as hydrogen halides, sulfuric acid, phosphoric acid, and the likes. Exemplary and non-limiting anions useful in pharmaceutically acceptable salts include oxalate, fumarate, maleate, acetate, propionate, succinate, tartrate, chloride, sulfate, bisulfate, mono-, di-, and tribasic phosphate, mesylate, tosylate, and the likes.

“Therapeutically effective amount” or dose of a compound or a composition refers to that amount of the compound or the composition that results in reduction or inhibition of symptoms or a prolongation of survival in a patient. The results may require multiple doses of the compound or the composition.

“Treatment” or “treating” refers to an approach for obtaining beneficial or desired results including clinical results. For purposes of this invention, beneficial or desired results include, but are not limited to, one or more of the following: decreasing one or more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delaying or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a patient. Also encompassed by “treatment” is a reduction of pathological consequence of the disease or disorder. The methods of the invention contemplate any one or more of these aspects of treatment.

As used herein, “delaying” development of a disease means to defer, hinder, slow, retard, stabilize and/or postpone development of the disease and/or slowing the progression or altering the underlying disease process and/or course once it has developed. This delay can be of varying lengths of time, depending on the history of the disease and/or individual being treated. As is evident to one skilled in the art, a sufficient or significant delay can, in effect, encompass prevention, in that the individual does not develop clinical symptoms associated with the disease. A method that “delays” development of a disease is a method that reduces probability of disease development in a given time frame and/or reduces extent of the disease in a given time frame, when compared to not using the method, including stabilizing one or more symptoms resulting from the disease.

An individual who is “at risk” of developing a disease may or may not have detectable disease, and may or may not have displayed detectable disease prior to the treatment methods described herein. “At risk” denotes that an individual has one or more so-called risk factors, which are measurable parameters that correlate with development of a disease. An individual having one or more of these risk factors has a higher probability of developing the disease than an individual without these risk factor(s). These risk factors include, but are not limited to, age, sex, race, diet, history of previous disease, presence of precursor disease and genetic (i.e., hereditary) considerations. Compounds may, in some embodiments, be administered to a subject (including a human) who is at risk or has a family history of the disease or condition.

“Stereoisomer” or “stereoisomers” refer to compounds that differ in the stereogenicity of the constituent atoms such as, without limitation, in the chirality of one or more stereocenters or related to the cis or trans configuration of a carbon-carbon or carbon-nitrogen double bond. Stereoisomers include enantiomers and diastereomers.

“Alkyl” refers to monovalent saturated aliphatic hydrocarbyl groups having from 1 to 12 carbon atoms, preferably from 1 to 10 carbon atoms, and more preferably from 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—). Cₓ alkyl refers to an alkyl group having x number of carbon atoms.

“Alkylene” refers to a divalent saturated aliphatic hydrocarbyl group having from 1 to 12 carbon atoms, preferably from 1 to 10 carbon atoms, and more preferably from 1 to 6 carbon atoms. This term includes, by way of example, linear and branched hydrocarbyl groups such as methylene (—CH₂—), ethylene (—CH₂CH₂— or —CH(Me)-), propylene (—CH₂CH₂CH₂— or —CH(Me)CH₂—, or —CH(Et)-) and the like.

“Alkenyl” refers to straight or branched monovalent hydrocarbyl groups having from 2 to 6 carbon atoms and preferably 2 to 4 carbon atoms and having at least 1 and preferably from 1 to 2 sites of vinyl (>C═C<) unsaturation. Such groups are exemplified, for example, by vinyl, allyl, and but-3-en-1-yl. Included within this term are the cis and trans isomers or mixtures of these isomers. Cₓ alkenyl refers to an alkenyl group having x number of carbon atoms.

“Alkynyl” refers to straight or branched monovalent hydrocarbyl groups having from 2 to 6 carbon atoms and preferably 2 to 3 carbon atoms and having at least 1 and preferably from 1 to 2 sites of acetylenic (—C≡C—) unsaturation. Examples of such alkynyl groups include acetylenyl (—C≡CH), and propargyl (—CH₂C≡CH). Cₓ alkynyl refers to an alkynyl group having x number of carbon atoms.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Aryl” refers to a monovalent aromatic carbocyclic group of from 6 to 14 carbon atoms having a single ring (e.g., phenyl (Ph)) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

“Cyano” refers to the group —C≡N.

“Cycloalkyl” refers to saturated or unsaturated but nonaromatic cyclic alkyl groups of from 3 to 10 carbon atoms, preferably from 3 to 8 carbon atoms, and more preferably from 3 to 6 carbon atoms, having single or multiple cyclic rings including fused, bridged, and spiro ring systems. Cₓ cycloalkyl refers to a cycloalkyl group having x number of ring carbon atoms. Examples of suitable cycloalkyl groups include, for instance, adamantyl, cyclopropyl, cyclobutyl, cyclopentyl, and cyclooctyl. One or more the rings can be aryl, heteroaryl, or heterocyclic provided that the point of attachment is through the non-aromatic, non-heterocyclic ring saturated carbocyclic ring. “Substituted cycloalkyl” refers to a cycloalkyl group having from 1 to 5 or preferably 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Halo” or “halogen” refers to fluoro, chloro, bromo and iodo and preferably is fluoro or chloro.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include 5 or 6 membered heteroaryls such as pyridinyl, pyrrolyl, thiophenyl, and furanyl. Other preferred heteroaryls include 9 or 10 membered heteroaryls, such as indolyl, quinolinyl, quinolonyl, isoquinolinyl, and isoquinolonyl.

“Heterocycle” or “heterocyclic” or “heterocycloalkyl” or “heterocyclyl” refers to a saturated or partially saturated, but not aromatic, group having from 1 to 10 ring carbon atoms, preferably from 1 to 8 carbon atoms, and more preferably from 1 to 6 carbon atoms, and from 1 to 4 ring heteroatoms, preferably from 1 to 3 heteroatoms, and more preferably from 1 to 2 heteroatoms selected from the group consisting of nitrogen, sulfur, or oxygen. Cₓ heterocycloalkyl refers to a heterocycloalkyl group having x number of ring atoms including the ring heteroatoms. Heterocycle encompasses single ring or multiple condensed rings, including fused bridged and spiro ring systems. In fused ring systems, one or more the rings can be cycloalkyl, aryl or heteroaryl provided that the point of attachment is through the non-aromatic ring. In one embodiment, the nitrogen and/or sulfur atom(s) of the heterocyclic group are optionally oxidized to provide for the N-oxide, sulfinyl, sulfonyl moieties.

Examples of heterocyclyl and heteroaryl include, but are not limited to, azetidinyl, pyrrolyl, imidazolyl, pyrazolyl, pyridyl, pyrazyl, pyrimidyl, pyridazyl, indolizyl, isoindolyl, indolyl, dihydroindolyl, indazolyl, purinyl, quinolizinyl, isoquinolinyl, quinolinyl, phthalazinyl, naphthylpyridinyl, quinoxalinyl, quinazolinyl, cinnolinyl, pteridinyl, carbazolyl, carbolinyl, phenanthridinyl, acridinyl, phenanthrolinyl, isothiazolyl, phenazinyl, isoxazolyl, phenoxazinyl, phenothiazinyl, imidazolidinyl, imidazolinyl, piperidinyl, piperazinyl, indolinyl, phthalimidyl, 1,2,3,4-tetrahydroisoquinolinyl, 4,5,6,7-tetrahydrobenzo[b]thiophenyl, thiazolyl, thiazolidinyl, thiophenyl, benzo[b]thiophenyl, morpholinyl, thiomorpholinyl (also referred to as thiamorpholinyl), 1,1-dioxothiomorpholinyl, piperidinyl, pyrrolidinyl, and tetrahydrofuranyl.

“Oxo” refers to the atom (═O) or (O).

The terms “optional” or “optionally” as used throughout the specification means that the subsequently described event or circumstance may but need not occur, and that the description includes instances where the event or circumstance occurs and instances in which it does not. For example, “the nitrogen atom is optionally oxidized to provide for the N-oxide (N→O) moiety” means that the nitrogen atom may but need not be oxidized, and the description includes situations where the nitrogen atom is not oxidized and situations where the nitrogen atom is oxidized.

The dosage amount of a compound as described herein is determined based on the free acid or free base of a compound, as appropriate.

### SSAO Inhibitors

Suitable SSAO inhibitors that can be used in accordance with the methods described herein include, but are not limited to PXS-4728A (BI-1467335) and a compound of formula (I) or a pharmaceutically acceptable salt. The compounds of formula (I), including Compound 1, is disclosed in US 2018/0297987, the content of which is incorporated by reference in its entirety, and specifically with respect to the compound of formula (I) or a pharmaceutically acceptable salt or enantiomer thereof, as well as methods of making and using the foregoing.

In some embodiments, the SSAO inhibitor is a compound of Formula (I)

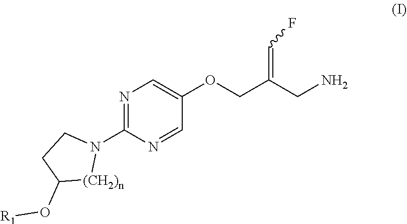

or a pharmaceutically acceptable salt thereof, wherein:
n is 1 or 2; and

### R1 is H or —CH₃.

The bond to fluorine, which is illustrated as [symbol] , indicates that the fluorine atom and the methoxypyrimidine group can be either Z (zusammen, together) or E (entgegen, opposite) relative to each other (Brecher, J., et al., “Graphical Representation of Stereochemical Configuration”, Pure and Appl. Chem, 2006, 78(10) 1897, at 1959). The structure illustrated by Formula (II) includes compounds with the Z stereochemical configuration, the E stereochemical configuration, or a mixture of compounds in the Z or E stereochemical configurations. Preferred compounds of the invention have the E stereochemical configuration.

In one form, the compounds of Formula (I) are presented as a free base. In other form, the compounds of Formula (I) are presented as acid addition salts, such as a mono or di HCl addition salt(s) or a sulfonate salt, preferably a 4-methylbenzenesulfonate (a tosylate salt).

In some embodiments, the SSAO inhibitor is a compound of formula (Ia)

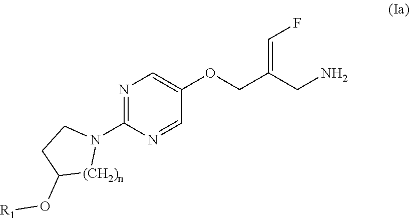

or a pharmaceutically acceptable salt thereof, wherein:
n is 1 or 2; and

### R1 is H or —CH₃.

In some embodiments, the SSAO inhibitor is

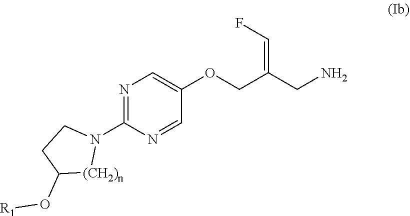

or a pharmaceutically acceptable salt thereof, wherein:
n is 1 or 2; and

### R1 is H or —CH₃.

In some embodiments, the SSAO inhibitor is a compound of formula (I), (Ia) or (Ib) and n is 2.

In some embodiments, the SSAO inhibitor is a compound of formula (I), (Ia) or (Ib) and R1 is CH₃.

In some embodiments, the SSAO inhibitor is a compound of formula 1:

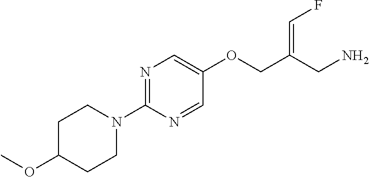 [SMILES: COC1CCN(c2ncc(OC/C(=C/F)CN)cn2)CC1]

or a pharmaceutically acceptable salt thereof, such as a dihydrochloride salt or a 4-methylbenzenesulfonate salt. “Compound 1” refers to the compound of formula 1.

### THR-β Agonists

Suitable THR-β agonists that can be used in accordance with the methods described herein include, but are not limited to resmetirom (MGL-3196), VK2809 (by Viking Therapeutics), sobetirome, eprotirome, ALG-055009 (by Aligo), CNPT-101101 (by FronThera Pharmaceuticals), CNPT-101207 (by FronThera Pharmaceuticals), ASC41 (Ascletis), and a compound of formula (II) or a pharmaceutically acceptable salt.

The compounds of formula (II) are disclosed in US Application Publication No. 20200190064, the contents of which are incorporated by reference in their entirety, and specifically with respect to the compounds of formula (II), such as compound 2, or a pharmaceutically acceptable salt or enantiomer thereof, as well as methods of making and using the foregoing.

In some embodiments, the THR-β agonist is a compound of Formula (II)

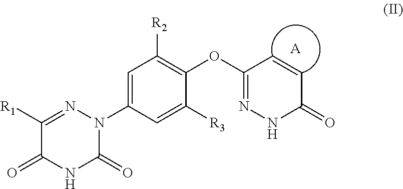

wherein:
R₁ is selected from the group consisting of hydrogen, cyano, substituted or unsubstituted C₁₋₆ alkyl, and substituted or unsubstituted C₃₋₆ cycloalkyl, the substituent being selected from the group consisting of halogen atoms, hydroxy, and C₁₋₆ alkoxy;
R₂ and R₃ are each independently selected from the group consisting of halogen atoms and substituted or unsubstituted C₁₋₆ alkyl, the substituent being selected from the group consisting of halogen atoms, hydroxy, and C₁₋₆ alkoxy;
ring A is a substituted or unsubstituted saturated or unsaturated C₅₋₁₀ aliphatic ring, or a substituted or unsubstituted C₅₋₁₀ aromatic ring, the substituent being one or more substances selected from the group consisting of hydrogen, halogen atoms, hydroxy, —OCF₃, —NH₂, —NHC₁₋₄ alkyl, —N(C₁₋₄ alkyl)₂, —CONH₂, —CONHC₁₋₄ alkyl, —CON(C₁₋₄ alkyl)₂, —NHCOC₁₋₄ alkyl, C₁₋₆ alkyl, C₁₋₆ alkoxy and C₃₋₆ cycloalkyl, and when two substituents are contained, the two substituents can form a ring structure together with the carbon connected thereto; and the halogen atoms are selected from the group consisting of F, Cl and Br,
or a pharmaceutically acceptable salt thereof.

In some embodiments, the THR-β agonist is a compound of Formula (IIa)

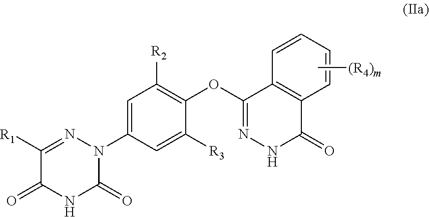

wherein:
R₁ to R₃ are defined as detailed herein for Formula (II);
R₄ is selected from the group consisting of hydrogen, halogen atoms, hydroxy, —OCF₃, —NH₂, —NHC₁₋₄ alkyl, —N(C₁₋₄ alkyl)₂, —CONH₂, —CONHC₁₋₄ alkyl, —CON(C₁₋₄ alkyl)₂, —NHCOC₁₋₄ alkyl, C₁₋₆ alkyl, C₁₋₆ alkoxy and C₃₋₆ cycloalkyl;
m is an integer from the range 1 to 4; and
   the halogen atoms are selected from the group consisting of F, Cl and Br.
   or a pharmaceutically acceptable salt thereof.

In some embodiments, wherein R₄ is selected from the group consisting of hydrogen, halogen atoms, hydroxy, —OCF₃, C₁₋₆ alkyl, C₁₋₆ alkoxy and C₃₋₆ cycloalkyl; and m is an integer from the range 1 to 3.

In some embodiments, wherein R₁ is selected from the group consisting of hydrogen, cyano, and substituted or unsubstituted C₁₋₆ alkyl, the substituent being selected from the group consisting of halogen atoms, hydroxy, and C₁₋₆ alkoxy; and the halogen atoms are selected from the group consisting of F, Cl and Br.

In some embodiments, the THR-β agonist is a compound of formula 2:

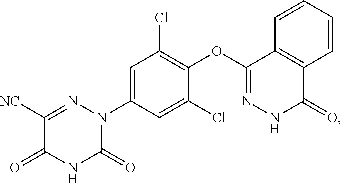 [SMILES: N#Cc1nn(-c2cc(Cl)c(Oc3n[nH]c(=O)c4ccccc34)c(Cl)c2)c(=O)[nH]c1=O]

or a pharmaceutically acceptable salt thereof, such as a potassium or sodium salt. “Compound 2” refers to the compound of formula 2.

### FXR Agonists

Suitable FXR agonists that can be used in accordance with the methods described herein include, but are not limited to obeticholic acid, cilofexor, tropifexor, EYP001 (Vonafexor, proposed INN), MET409 (Metacrine), EDP-305 (by Enanta) and a compound of formula (I) or a pharmaceutically acceptable salt. The compounds of formula (III), including Compound 3, are disclosed in US 2010/0152166, the content of which is incorporated by reference in its entirety, and specifically with respect to the compound of formula (I) or a pharmaceutically acceptable salt or enantiomer thereof, as well as methods of making and using the foregoing.

In some embodiments, the FXR agonist is a compound of formula (III)

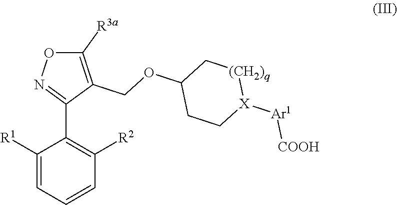

wherein:
q is 1 or 2;
R¹ is chloro, fluoro, or trifluoromethoxy;
R² is hydrogen, chloro, fluoro, or trifluoromethoxy;
R^{3a} is trifluoromethyl, cyclopropyl, or isopropyl;

### X is CH or N,

provided that when X is CH, q is 1; and
Ar¹ is indolyl, benzothienyl, naphthyl, phenyl, benzoisothiazolyl, indazolyl, or pyridinyl, each of which is optionally substituted with methyl or phenyl,
or a pharmaceutically acceptable salt thereof.

In some embodiments, the FXR agonist is a compound of formula (III), wherein R¹ is chloro or trifluoromethoxy; and R² is hydrogen or chloro.

In some embodiments, the FXR agonist is a compound of formula (III), wherein R^{3a} is cyclopropyl or isopropyl.

In some embodiments, the FXR agonist is a compound of formula (III), wherein Ar¹ is 5-benzothienyl, 6-benzothienyl, 5-indolyl, 6-indolyl, or 4-phenyl, each of which is optionally substituted with methyl.

In some embodiments, the FXR agonist is a compound of formula (III), wherein q is 1; and X is N.

In some embodiments, the FXR agonist is a compound of formula 3:

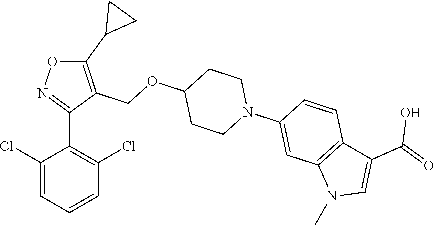 [SMILES: Cn1cc(C(=O)O)c2ccc(N3CCC(OCc4c(-c5c(Cl)cccc5Cl)noc4C4CC4)CC3)cc21]

or a pharmaceutically acceptable salt thereof “Compound 3” refers to the compound of formula 3.

### Pharmaceutically Acceptable Compositions and Formulations

Pharmaceutically acceptable compositions or simply “pharmaceutical compositions” of any of the compounds detailed herein are embraced by this invention. Thus, the invention includes pharmaceutical compositions comprising an SSAO inhibitor (such as the compound of Formula (I) or a pharmaceutically acceptable salt thereof), a THR-β agonist (such as the compounds of Formula (II) or a pharmaceutically acceptable salt thereof), and a pharmaceutically acceptable carrier or excipient. In some embodiments, the pharmaceutically acceptable salt is an acid addition salt, such as a salt formed with an inorganic or organic acid. Pharmaceutical compositions according to the invention may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. In one variation, “substantially pure” intends a composition that contains no more than 35% impurity, wherein the impurity denotes a compound other than the compound comprising the majority of the composition or a salt thereof. For example, a composition of a substantially pure compound intends a composition that contains no more than 35% impurity, wherein the impurity denotes a compound other than the compound or a salt thereof. In one variation, a composition of substantially pure compound or a salt thereof is provided wherein the composition contains no more than 25% impurity. In another variation, a composition of substantially pure compound or a salt thereof is provided wherein the composition contains or no more than 20% impurity. In still another variation, a composition of substantially pure compound or a salt thereof is provided wherein the composition contains or no more than 10% impurity. In a further variation, a composition of substantially pure compound or a salt thereof is provided wherein the composition contains or no more than 5% impurity. In another variation, a composition of substantially pure compound or a salt thereof is provided wherein the composition contains or no more than 3% impurity. In still another variation, a composition of substantially pure compound or a salt thereof is provided wherein the composition contains or no more than 1% impurity. In a further variation, a composition of substantially pure compound or a salt thereof is provided wherein the composition contains or no more than 0.5% impurity. In yet other variations, a composition of substantially pure compound means that the composition contains no more than 15% or preferably no more than 10% or more preferably no more than 5% or even more preferably no more than 3% and most preferably no more than 1% impurity, which impurity may be the compound in a different stereochemical form.

In one variation, the compounds herein are synthetic compounds prepared for administration to an individual such as a human. In another variation, compositions are provided containing a compound in substantially pure form. In another variation, the invention embraces pharmaceutical compositions comprising a compound detailed herein and a pharmaceutically acceptable carrier or excipient. In another variation, methods of administering a compound are provided. The purified forms, pharmaceutical compositions and methods of administering the compounds are suitable for any compound or form thereof detailed herein.

The compounds may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

Compounds described herein can be used in the preparation of a formulation, such as a pharmaceutical formulation, by combining the compounds as active ingredients with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington: The Science and Practice of Pharmacy, Lippincott Williams & Wilkins, 21^{st} ed. (2005), which is incorporated herein by reference.

Compounds as described herein may be administered to individuals (e.g., a human) in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid polyols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Compositions comprising two compounds (an SSAO inhibitor and a THR-β agonist) or three compounds (an SSAO inhibitor, a THR-β agonist, and an FXR agonist) utilized herein are described. Any of the compounds described herein can be formulated in a tablet in any dosage form described herein.

The present disclosure further encompasses kits (e.g., pharmaceutical packages). The kit provided may comprise the pharmaceutical compositions or the compounds described herein and containers (e.g., drug bottles, ampoules, bottles, syringes and/or subpackages or other suitable containers). In some embodiments, the kit includes a container comprising the SSAO inhibitor (such as the compound of Formula (I) or a pharmaceutically acceptable salt thereof) and the THR-β agonist (such as the compound of (II) or a pharmaceutically acceptable salt thereof). In some such embodiments, the container further comprises the FXR agonist (such as the compound of Formula (III) or a pharmaceutically acceptable salt thereof). In other embodiments, the kit includes a first container comprising SSAO inhibitor (such as the compound of Formula (I) or a pharmaceutically acceptable salt thereof) and a second container comprising the THR-β agonist (such as the compound of (II) or a pharmaceutically acceptable salt thereof). In some such embodiments, the kit further includes a third container comprising FXR agonist (such as the compound of Formula (III) or a pharmaceutically acceptable salt thereof).

In some embodiments, the composition comprises the SSAO inhibitor and the THR-β agonist as described herein. In some embodiments, such a composition includes a compound of formula (I), or a pharmaceutically acceptable salt thereof, and a compound of formula (II), or a pharmaceutically acceptable salt thereof. In some embodiments, provided herein is a dosage form comprises a therapeutically effective amount of a compound of formula (I), or a pharmaceutically acceptable salt thereof, and a therapeutically effective amount of a compound of formula (II), or a pharmaceutically acceptable salt thereof. In some embodiments, the compound of formula (I), or a pharmaceutically acceptable salt thereof, is Compound 1 or a pharmaceutically acceptable salt thereof, and the compound of formula (II), or a pharmaceutically acceptable salt thereof, is Compound 2 or a pharmaceutically acceptable salt thereof, as described herein. In some embodiments of the foregoing, the composition further comprises the FXR agonist as described herein. In some such embodiments, the FXR agonist is a compound of formula (III), or a pharmaceutically acceptable salt thereof. In some embodiments, the compound of formula (III) is Compound 3 or a pharmaceutically acceptable salt thereof, as described herein.

### Methods of Use and Uses

Compounds and compositions described herein may in some aspects be used in treatment or prevention of liver disorders. In some embodiments, the method of treating or preventing a liver disorder in a patient in need thereof comprises administering to the patient a Semicarbazide-Sensitive Amine Oxidase (SSAO) inhibitor and a thyroid hormone receptor beta (THR-β) agonist. In some embodiments, the method further comprises administering to the patient an FXR agonist, including but not limited to Compound 3. In some embodiments, the SSAO inhibitor is a compound of Formula (I), or a pharmaceutically acceptable salt thereof, and the THR-β agonist is a compound of Formula (II), or a pharmaceutically acceptable salt thereof. In one embodiment, the compound of Formula (I), or a pharmaceutically acceptable salt thereof, is Compound 1, and the compound of Formula (II), or a pharmaceutically acceptable salt thereof, is Compound 2 as described herein. Without being bound by theory, it is believed that the combination of an SSAO inhibitor and a THR-β agonist in accordance with the methods described herein may effectively provide treatment as compared to monotherapies and thus reduce dose-dependent adverse effects that may accompany monotherapy treatment.

Liver disorders include, without limitation, liver inflammation, fibrosis, and steatohepatitis. In some embodiments, the liver disorder is selected from liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH). In certain embodiments, the liver disorder is selected from: liver fibrosis, alcohol induced fibrosis, steatosis, alcoholic steatosis, NAFLD, and NASH. In one embodiment, the liver disorder is NASH. In another embodiment, the liver disorder is liver inflammation. In another embodiment, the liver disorder is liver fibrosis. In another embodiment, the liver disorder is alcohol induced fibrosis. In another embodiment, the liver disorder is steatosis. In another embodiment, the liver disorder is alcoholic steatosis. In another embodiment, the liver disorder is NAFLD. In one embodiment, the treatment methods provided herein impedes or slows the progression of NAFLD to NASH. In one embodiment, the treatment methods provided herein impedes or slows the progression of NASH. NASH can progress, e.g., to one or more of liver cirrhosis, hepatic cancer, etc. In some embodiments, the liver disorder is NASH. In some embodiments, the patient has had a liver biopsy. In some embodiments, the method further comprising obtaining the results of a liver biopsy.

In some embodiments, the method of treating a liver disorder in a patient in need thereof, wherein the liver disorder is selected from the group consisting of liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH).

Provided herein are methods of treating or preventing a liver disorder in a patient (e.g., a human patient) in need thereof with an SSAO inhibitor and a THR-β agonist, comprising administering a therapeutically effective amount of the SSAO inhibitor and a therapeutically effective amount of the THR-β agonist, wherein the liver disorder is selected from liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH). In some embodiments, the method further comprises an FXR agonist, including but not limited to Compound 3. In some embodiments, the SSAO inhibitor is a compound of Formula (I) or a pharmaceutically acceptable salt thereof and the THR-β agonist is a compound of formula (II) or a pharmaceutically acceptable salt thereof. In some embodiments, the compound of formula (I), or a pharmaceutically acceptable salt thereof, is Compound 1, and the compound of formula (II), or a pharmaceutically acceptable salt thereof, is Compound 2 as described herein.

Also provided herein are methods of impeding or slowing the progression of non-alcoholic fatty liver disease (NAFLD) to non-alcoholic steatohepatitis (NASH) in a patient (e.g., a human patient) in need thereof comprising administering an SSAO inhibitor (such as the compound of Formula (I) or a pharmaceutically acceptable salt thereof) and a THR-β agonist (such as the compounds of Formula (II) or a pharmaceutically acceptable salt thereof). In some embodiments, the method further comprises administering an FXR agonist, including but not limited to Compound 3. In some embodiments, the methods comprise administering a therapeutically effective amount of a compound of formula (I), or a pharmaceutically acceptable salt thereof, and a therapeutically effective amount of a compound of formula (II) or a pharmaceutically acceptable salt thereof, and optionally a therapeutically effective amount of a compound of formula (III) or a pharmaceutically acceptable salt thereof. Also provided herein are methods of impeding or slowing the progression of NASH in a patient (e.g., a human patient) in need thereof comprising administering an SSAO inhibitor (such as the compound of Formula (I) or a pharmaceutically acceptable salt thereof) and a THR-β agonist (such as the compounds of Formula (II) or a pharmaceutically acceptable salt thereof). In some embodiments, the method further comprises administering an FXR agonist, including but not limited to Compound 3. In some embodiments, the methods comprise administering a therapeutically effective amount of a compound of formula (I), or a pharmaceutically acceptable salt thereof, and a therapeutically effective amount of a compound of formula (II) or a pharmaceutically acceptable salt thereof, and optionally a therapeutically effective amount of a compound of formula (III) or a pharmaceutically acceptable salt thereof.

In a patient, alkaline phosphatase, gamma-glutamyl transferase (GGT), alanine aminotransferase (ALT) and/or aspartate aminotransferase (AST) levels can be elevated. In some embodiments, provided herein a method of reducing liver damage comprising administering an SSAO inhibitor (such as the compound of Formula (I) or a pharmaceutically acceptable salt thereof) and a THR-β agonist (such as the compounds of Formula (II) or a pharmaceutically acceptable salt thereof), wherein the GGT, ALT, and/or AST levels are elevated prior to treatment with the SSAO inhibitor. In some embodiments, the SSAO inhibitor is a compound of Formula (I) or a pharmaceutically acceptable salt thereof.

In some embodiments, provided herein are methods of treating a liver disorder in a patient in need thereof with an SSAO inhibitor (such as the compound of Formula (I) or a pharmaceutically acceptable salt thereof) and a THR-β agonist (such as the compound of Formula (II) or a pharmaceutically acceptable salt thereof), wherein the SSAO inhibitor selectively inhibits SSAO. In some embodiments, the SSAO inhibitor is a compound of Formula (I) or a pharmaceutically acceptable salt thereof. Accordingly, in some embodiments, MAO-A (Monoamine oxidase A) is not inhibited. In some embodiments, MAO-B (Monoamine oxidase B) is not inhibited. In some embodiments MAO-A and MAO-B are not inhibited.

In some embodiments, the patient is a human. Obesity is highly correlated with NAFLD and NASH, but lean people can also be affected by NAFLD and NASH. Accordingly, in some embodiments, the patient is obese. In some embodiments, the patient is not obese. Obesity can be correlated with or cause other diseases as well, such as diabetes mellitus or cardiovascular disorders. Accordingly, in some embodiments, the patient also has diabetes mellitus and/or a cardiovascular disorder. Without being bound by theory, it is believed that comorbidities, such as obesity, diabetes mellitus, and cardiovascular disorders can make NAFLD and NASH more difficult to treat. Conversely, the only currently recognized method for addressing NAFLD and NASH is weight loss, which would likely have little to no effect on a lean patient.

The risk for NAFLD and NASH increases with age, but children can also suffer from NAFLD and NASH, with literature reporting of children as young as 2 years old (Schwimmer, et al., Pediatrics, 2006, 118:1388-1393). In some embodiments, the patient is 2-17 years old, such as 2-10, 2-6, 2-4, 4-15, 4-8, 6-15, 6-10, 8-17, 8-15, 8-12, 10-17, or 13-17 years old. In some embodiments, the patient is 18-64 years old, such as 18-55, 18-40, 18-30, 18-26, 18-21, 21-64, 21-55, 21-40, 21-30, 21-26, 26-64, 26-55, 26-40, 26-30, 30-64, 30-55, 30-40, 40-64, 40-55, or 55-64 years old. In some embodiments, the patient is 65 or more years old, such as 70 or more, 80 or more, or 90 or more.

NAFLD and NASH are common causes of liver transplantation, but patients that already received one liver transplant often develop NAFLD and/or NASH again. Accordingly, in some embodiments, the patient has had a liver transplant.

In some embodiments, treatment in accordance with the methods provided herein results in a reduced NAFLD Activity (NAS) score in a patient. For example, in some embodiments, steatosis, inflammation, and/or ballooning is reduced upon treatment. In some embodiments, the methods of treatment provided herein reduce liver fibrosis. In some embodiments, the methods reduce serum triglycerides. In some embodiments, the methods reduce liver triglycerides.

In some embodiments, the patient is at risk of developing an adverse effect prior to the administration in accordance with the methods provided herein. In some embodiments, the adverse effect is an adverse effect which affects the kidney, lung, heart, and/or skin.

In some embodiments, the patient has had one or more prior therapies. In some embodiments, the liver disorder progressed during the therapy.

In some embodiments, the methods do not comprise administering an antihistamine, an immunosuppressant, a steroid (such as a corticosteroid), rifampicin, an opioid antagonist, or a selective serotonin reuptake inhibitor (SSRI).

In some embodiments, the SSAO inhibitor and the THR-β agonist, and optionally the FXR agonist, are administered simultaneously. In some such embodiments, the SSAO inhibitor and the THR-β agonist, and optionally the FXR agonist, can be provided in a single pharmaceutical composition. In other embodiments, the SSAO inhibitor and the THR-β agonist, and optionally the FXR agonist, are administered sequentially.

When administered in combination with a THR-β agonist, the SSAO inhibitor and/or the THR-β agonist and/or the optional FXR agonist may be administered at doses that are typically administered when either agent is administered alone. Alternatively, as a result of the likely synergy observed with the combination, the SSAO and/or the THR-β agonist and/or the optional FXR agonist may be administered at doses that are lower than doses when each agent is administered alone.

In embodiments wherein the SSAO inhibitor is Compound 1 or a pharmaceutically acceptable salt thereof, a therapeutic dose of the compound to a human patient is typically from about 4 mg to about 40 mg daily administered orally. In particular embodiments, when administered in combination with a THR-β agonist, optionally further in combination with an FXR agonist, the compound of formula (I) or a pharmaceutically acceptable salt thereof may be administered at an oral dose of from about 4 mg to about 20 mg (e.g., 4 mg, 5 mg, 6 mg, 8 mg, 10 mg, 15 mg, or 20 mg) or may be administered at a lower dose. For instance, when administered in combination with a THR-β agonist, optionally further in combination with an FXR agonist, the compound of formula (I) or a pharmaceutically acceptable salt thereof may be administered orally at a dose of from about 1 mg to about 40 mg daily, from about 1 mg to about 20 mg daily, from about 1 mg to about 3.9 mg daily, from about 1 mg to about 3 mg daily, from about 1.5 mg to about 3.5 mg daily, from about 2 mg to about 3 mg daily, or any of 1, 1.5, 2, 2.5, 3, 3.5, 3.6, 3.8, 3.9, 4, 5, 6, 7, 8, 9, 10, 11, 12, 13, 14, 15, 16, 17, 18, 19, or 20 mg daily.

In embodiments wherein the THR-β agonist is Compound 2 or a pharmaceutically acceptable salt thereof, a therapeutic dose of each compound to a human patient is typically from about 0.5 mg to about 90 mg daily, from about 1 mg to about 90 mg daily, from about 3 mg to about 90 mg daily, from about 0.5 mg to about 30 mg daily, from about 1 mg to about 30 mg daily, or from about 3 mg to about 30 mg daily administered orally. In particular embodiments, when administered in combination with an SSAO inhibitor, optionally further in combination with an FXR agonist, the compound of formula (II) or a pharmaceutically acceptable salt thereof may be administered at an oral dose of from about 0.5 mg to about 90 mg (e.g., 0.5 mg, 1 mg, 3 mg, 5 mg, 6 mg, 10 mg, 20 mg, 30 mg, 40 mg, 50 mg, 60 mg, 70 mg, 80 mg or 90 mg) or can be administered at a lower dose. For instance, when administered in combination with an SSAO inhibitor, optionally further in combination with an FXR agonist, the compound of formula (II) or a pharmaceutically acceptable salt thereof may be administered orally at a dose of from about 0.1 mg to about 90 mg daily, from about 0.1 mg to about 30 mg daily, from about 0.1 mg to about 25 mg daily, from about 0.1 mg to about 20 mg daily, from about 0.1 mg to about 15 mg daily, from about 0.1 mg to about 10 mg daily, from about 0.1 mg to about 5 mg daily, from about 0.1 mg to about 3 mg daily, or from about 0.1 mg to about 1 mg daily.

In embodiments wherein the FXR agonist is Compound 3 or a pharmaceutically acceptable salt thereof, a therapeutic dose of the compound to a human patient is typically from about 5 mg to about 75 mg daily, or from about 5 mg to about 25 mg daily, preferably from about 10 mg to about 15 mg daily, administered orally. In particular embodiments, when administered in combination with an SSAO inhibitor and a THR-β agonist, the compound of formula (III) or a pharmaceutically acceptable salt thereof may be administered at an oral dose of from about 5 mg to about 75 mg or can be administered at a lower dose. For instance, when administered in combination with an SSAO inhibitor and a THR-β agonist, the compound of formula (III) or a pharmaceutically acceptable salt thereof may be administered orally at a dose of from about 1 mg to about 75 mg daily, from about 5 mg to about 75 mg daily, from about 1 mg to about 25 mg daily, from about 1 mg to about 15 mg daily, from about 1 mg to about 10 mg daily, from about 1 mg to about 5 mg daily, or from about 5 mg to about 10 mg daily.

In particular embodiments wherein the SSAO inhibitor is a compound of formula (I) (e.g., Compound 1) or a pharmaceutically acceptable salt thereof and the THR-β agonist is a compound of formula (II) (e.g., Compound 2) or a pharmaceutically acceptable salt thereof, optionally further in combination with an FXR agonist, the dose of each individual compound may be administered as set forth above. For instance, in some embodiments, Compound 1 or a pharmaceutically acceptable salt thereof may be administered at a dose from about 1 mg to about 40 mg daily, in combination with Compound 2 or a pharmaceutically acceptable salt thereof administered at a dose of from about 0.1 mg to about 90 mg daily, optionally in combination with Compound 3 or a pharmaceutically acceptable salt thereof administered at a dose of from about 1 mg to about 75 mg daily. In some embodiments, Compound 1 or a pharmaceutically acceptable salt thereof may be administered at a dose of from about 1 mg to about 20 mg daily, in combination with Compound 2 or a pharmaceutically acceptable salt thereof administered at a dose of from about 0.1 mg to about 30 mg daily, optionally in combination with Compound 3 or a pharmaceutically acceptable salt thereof administered at a dose of from about 1 mg to about 20 mg daily. In some embodiments, Compound 1 or a pharmaceutically acceptable salt thereof may be administered at a dose of from about 1 mg to about 10 mg daily, in combination with Compound 2 or a pharmaceutically acceptable salt thereof administered at a dose of from about 0.1 mg to about 10 mg daily, optionally in combination with Compound 3 or a pharmaceutically acceptable salt thereof administered at a dose of from about 1 mg to about 15 mg daily. In some embodiments, Compound 1 or a pharmaceutically acceptable salt thereof may be administered at a dose of from about 1 mg to about 6 mg daily, in combination with Compound 2 or a pharmaceutically acceptable salt thereof administered at a dose of from about 0.1 mg to about 5 mg daily, optionally in combination with Compound 3 or a pharmaceutically acceptable salt thereof administered at a dose of from about 1 mg to about 10 mg daily.

In some embodiments, the compound of formula (I) or a pharmaceutically acceptable salt thereof may be administered at a dose from about 5 mg to about 15 mg daily, in combination with the compound of formula (II) or a pharmaceutically acceptable salt thereof administered at a dose of from about 0.1 mg to about 10 mg daily, from about 10 mg to about 20 mg daily, from about 10 mg to about 40 mg daily, from about 20 mg to about 50 mg daily or from about 50 mg to about 90 mg daily. In some embodiments, the compound of formula (I) or a pharmaceutically acceptable salt thereof may be administered at a dose from about 1 mg to about 5 mg daily, in combination with the compound of formula (II) or a pharmaceutically acceptable salt thereof administered at a dose of from about 0.1 mg to about 10 mg daily, from about 10 mg to about 20 mg daily, from about 10 mg to about 40 mg daily, from about 20 mg to about 50 mg daily or from about 50 mg to about 90 mg daily.

In some embodiments, the amount of the SSAO inhibitor (such as the compound of Formula (I) or a pharmaceutically acceptable salt thereof) and the amount of the THR-β agonist (such as the compound of Formula (II) or a pharmaceutically acceptable salt thereof), and optionally the amount of the FXR agonist, administered on day 1 of the treatment period may be greater than or equal to the amount of said compounds administered on all subsequent days of the treatment period. In some embodiments, the amounts of each compound administered on day 1 of the treatment period may be equal to the amounts of said compound administered on all subsequent days of the treatment period.

A compound of Formula (I), or a pharmaceutically acceptable salt thereof, used in accordance with the method described herein, can be administered to an individual a once daily dose for a first period of time, followed by a second period of time in which administration of the compound may be discontinued, wherein the SSAO inhibitory activity may be maintained during both the first and the second period of time. In some embodiments, the first and second periods of time are each one-week periods. For example, provided herein is a method of treatment in an individual for a period of 14 days comprising administering to the individual a once daily dose of a compound of Formula (I), or a pharmaceutically acceptable salt thereof, for a first 7 days, followed by discontinued administration of the compound for the following 7 days, wherein the SSAO inhibitory activity may be maintained in the individual during the entire 14-day period. As another example, provided herein is a method of treatment in an individual for a period of four weeks, comprising administering to the individual a once daily dose of a compound of Formula (I), or a pharmaceutically acceptable salt thereof, for a first two weeks, followed by discontinued administration of the compound for the following two weeks, wherein the SSAO inhibitory activity may be maintained in the individual during the entire four-week period. In some embodiments, the daily dose is about 10 mg. It is understood that the dosages and dosing regimens disclosed herein are also applicable in a monotherapy for treating NASH using a compound of Formula (I), or a pharmaceutically acceptable salt thereof.

In some embodiments, administration with the combination of the SSAO inhibitor (such as the compound of Formula (I) or a pharmaceutically acceptable salt thereof) and the THR-β agonist (such as the compound of Formula (II) or a pharmaceutically acceptable salt thereof), and optionally the FXR agonist, decreases steatosis in the individual. Methods of assessing steatosis are known to the skilled artisan and may include histological analysis and assignment of histological score. In some embodiments, administration with the combination may decrease steatosis in the individual as compared to administration with a monotherapy of the SSAO inhibitor or the THR-β agonist. In some embodiments, administration with the combination may decrease steatosis in the individual comparably as well as administration with a monotherapy of the SSAO inhibitor or the THR-β agonist. In some embodiments, administration with the combination may provide a synergistic decrease in steatosis in the individual as compared to administration with a monotherapy of the SSAO inhibitor or the THR-β agonist. Thus it is understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises reducing histological markers associated with steatosis.

In some embodiments, administration with the combination of the SSAO inhibitor (such as the compound of Formula (I) or a pharmaceutically acceptable salt thereof) and the THR-β agonist (such as the compound of (II) or a pharmaceutically acceptable salt thereof), and optionally the FXR agonist, decreases liver inflammation in the individual. Methods of assessing liver inflammation are known to the skilled artisan and may include histological analysis and assignment of histological score of lobular inflammation. In some embodiments, administration with the combination may decrease liver inflammation in the individual as compared to administration with a monotherapy of the SSAO inhibitor or the THR-β agonist. In some embodiments, administration with the combination may decrease liver inflammation in the individual comparably as well as administration with a monotherapy of the SSAO inhibitor or the THR-β agonist. In some embodiments, administration with the combination may provide a synergistic decrease in liver inflammation in the individual as compared to administration with a monotherapy of the SSAO inhibitor or the THR-β agonist. Thus it is understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises reducing lobular inflammation or histological markers associated with lobular inflammation.

In some embodiments, administration with the combination of the SSAO inhibitor (such as the compound of Formula (I) or a pharmaceutically acceptable salt thereof) and the THR-β agonist (such as the compound of (II) or a pharmaceutically acceptable salt thereof), and optionally the FXR agonist, decreases liver fibrosis in the individual. Methods of assessing liver fibrosis are known to the skilled artisan and may include histological analysis. In some embodiments, administration with the combination may decrease liver fibrosis in the individual as compared to administration with a monotherapy of the SSAO inhibitor or the THR-β agonist. In some embodiments, administration with the combination may decrease liver fibrosis in the individual comparably as well as administration with a monotherapy of the SSAO inhibitor or the THR-β agonist. In some embodiments, administration with the combination may provide a synergistic decrease in liver fibrosis in the individual as compared to administration with a monotherapy of the SSAO inhibitor or the THR-β agonist. Thus it is understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises reducing fibrosis or histological markers associated with fibrosis.

In some embodiments, administration with the combination of the SSAO inhibitor (such as the compound of Formula (I) or a pharmaceutically acceptable salt thereof) and the THR-β agonist (such as the compound of (II) or a pharmaceutically acceptable salt thereof), and optionally the FXR agonist, decreases at least one or at least two of liver steatosis, inflammation, and fibrosis in the individual. In some embodiments, administration with the combination may decrease at least one or at least two of liver steatosis, inflammation, and fibrosis in the individual as compared to administration with a monotherapy of the SSAO inhibitor or the THR-β agonist. In some embodiments, administration with the combination decreases liver steatosis, inflammation, and fibrosis in the individual. In some embodiments, administration with the combination may decrease liver steatosis, inflammation, and fibrosis in the individual as compared to administration with a monotherapy of the SSAO inhibitor or the THR-β agonist. In some embodiments, administration with the combination may provide a synergistic decrease in at least one or at least two of steatosis, inflammation, and fibrosis in the individual as compared to administration with a monotherapy of the SSAO inhibitor or the THR-β agonist. In some embodiments, administration with the combination may provide a synergistic decrease in steatosis, inflammation, and fibrosis in the individual as compared to administration with a monotherapy of the SSAO inhibitor or the THR-β agonist. Thus it is understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises reducing at least one or at least two of steatosis, lobular inflammation, fibrosis, or histological markers of any of the foregoing.

In some embodiments, administration with the combination of the SSAO inhibitor (such as the compound of Formula (I) or a pharmaceutically acceptable salt thereof) and the THR-β agonist (such as the compound of (II) or a pharmaceutically acceptable salt thereof), and optionally the FXR agonist, decreases serum triglycerides in the individual. In some embodiments, administration with the combination may decrease serum triglycerides in the individual as compared to administration with a monotherapy of the SSAO inhibitor or the THR-β agonist. In some embodiments, administration with the combination may decrease serum triglycerides in the individual comparably as well as administration with a monotherapy of the SSAO inhibitor or the THR-β agonist. Thus it is understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises reducing serum triglycerides.

In some embodiments, administration with the combination of the SSAO inhibitor (such as the compound of Formula (I) or a pharmaceutically acceptable salt thereof) and the THR-β agonist (such as the compound of (II) or a pharmaceutically acceptable salt thereof), and optionally the FXR agonist, decreases serum total cholesterol in the individual. In some embodiments, administration with the combination may decrease serum total cholesterol in the individual as compared to administration with a monotherapy of the SSAO inhibitor or the THR-β agonist. In some embodiments, administration with the combination may decrease serum total cholesterol in the individual comparably as well as administration with a monotherapy of the SSAO inhibitor or the THR-β agonist. Thus it is understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises reducing serum cholesterol.

In some embodiments, administration with the combination of the SSAO inhibitor (such as the compound of Formula (I) or a pharmaceutically acceptable salt thereof) and the THR-β agonist (such as the compound of (II) or a pharmaceutically acceptable salt thereof), and optionally the FXR agonist, decreases serum alanine aminotransferase in the individual. In some embodiments, administration with the combination may decrease serum alanine aminotransferase in the individual as compared to administration with a monotherapy of the SSAO inhibitor or the THR-β agonist. In some embodiments, administration with the combination may decrease serum alanine aminotransferase in the individual comparably as well as administration with a monotherapy of the SSAO inhibitor or the THR-β agonist. Thus it is understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises reducing serum alanine aminotransferase.

In some embodiments, administration with the combination of the SSAO inhibitor (such as the compound of Formula (I) or a pharmaceutically acceptable salt thereof) and the THR-β agonist (such as the compound of (II) or a pharmaceutically acceptable salt thereof), and optionally the FXR agonist, decreases at least one or at least two of serum triglycerides, total cholesterol, and alanine aminotransferase in the individual. In some embodiments, administration with the combination may decrease at least one or at least two of serum triglycerides, total cholesterol, and alanine aminotransferase in the individual as compared to administration with a monotherapy of the SSAO inhibitor or the THR-β agonist. In some embodiments, administration with the combination may decrease serum triglycerides, total cholesterol, and alanine aminotransferase in the individual. In some embodiments, administration with the combination may decrease serum triglycerides, total cholesterol, and alanine aminotransferase in the individual as compared to administration with a monotherapy of the SSAO inhibitor or the THR-β agonist. Thus it is understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises reducing at least one or at least two of serum triglycerides, total cholesterol, and alanine aminotransferase.

In some embodiments, provided are methods of reducing hepatic inflammation in a patient in need thereof, comprising administering to the patient a combination of the SSAO inhibitor (such as the compound of Formula (I) or a pharmaceutically acceptable salt thereof) and the THR-β agonist (such as the compound of (II) or a pharmaceutically acceptable salt thereof), and optionally the FXR agonist. In some embodiments, the method does not increase LDL-c levels in the patient. In some embodiments, the method decreases LDL-c levels in the patient. In some embodiments, the patient has a disease characterized by liver inflammation. In some embodiments, the patient has liver fibrosis. In some embodiments, the patient has NASH.

In some embodiments, provided are methods of treating a disease characterized by fibrosis of the liver in a patient in need thereof, comprising administering to the patient a combination of the SSAO inhibitor (such as the compound of Formula (I) or a pharmaceutically acceptable salt thereof) and the THR-β agonist (such as the compound of (II) or a pharmaceutically acceptable salt thereof), and optionally the FXR agonist. In some embodiments, the disease is associated with hepatic inflammation. In some embodiments, the patient has NASH.

In some embodiments, provided are methods of inhibiting expression of genes responsible for the production of collagen in the extracellular matrix of the liver in a patient in need thereof, comprising administering to the patient a combination of the SSAO inhibitor (such as the compound of Formula (I) or a pharmaceutically acceptable salt thereof) and the THR-β agonist (such as the compound of (II) or a pharmaceutically acceptable salt thereof), and optionally the FXR agonist. In some embodiments, the genes are fibroblast genes. In some embodiments, the patient has liver fibrosis. In some embodiments, the patient has NASH.

Also provided herein are combinations of the SSAO inhibitor (such as the compound of Formula (I) or a pharmaceutically acceptable salt thereof) and the THR-β agonist (such as the compounds of Formula (II) or a pharmaceutically acceptable salt thereof), and optionally the FXR agonist, for use in treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, using the methods as described herein.

Also provided herein are uses of the combinations of the SSAO inhibitor (such as the compound of Formula (I) or a pharmaceutically acceptable salt thereof) and the THR-β agonist (such as the compounds of Formula (II) or a pharmaceutically acceptable salt thereof), and optionally the FXR agonist, for manufacture of a medicament for treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, using the methods as described herein.

In some embodiments of the foregoing, the SSAO inhibitor (such as the compound of Formula (I) or a pharmaceutically acceptable salt thereof) is administered orally. In some embodiments of the foregoing, the THR-β agonist (such as the compounds of Formula (II) or a pharmaceutically acceptable salt thereof) is administered orally. In some embodiments of the foregoing, the FXR agonist (such as the compounds of Formula (III) or a pharmaceutically acceptable salt thereof) is administered orally.

In some embodiments of any of the foregoing, the compound of Formula (I) is Compound 1 or a tosylate salt thereof, and the compound of Formula (II) is Compound 2 or a potassium salt thereof. In some such embodiments, the FXR agonist is a compound of Formula (III), preferably Compound 3 or a pharmaceutically acceptable salt thereof.

### Articles of Manufacture and Kits

The present disclosure further provides articles of manufacture comprising a compound described herein, or a salt thereof, a composition described herein, or one or more unit dosages described herein in suitable packaging. In certain embodiments, the article of manufacture is for use in any of the methods described herein. Suitable packaging (e.g., containers) is known in the art and includes, for example, vials, vessels, ampules, bottles, jars, flexible packaging and the like. An article of manufacture may further be sterilized and/or sealed.

The present disclosure further provides kits for carrying out the methods of the present disclosure, which comprises at least two compounds described herein, or a pharmaceutically acceptable salt thereof, or a composition comprising a compound described herein, or a pharmaceutically acceptable salt thereof. The kits may employ any of the compounds disclosed herein or a pharmaceutically acceptable salt thereof. In some embodiments, the kit employs an SSAO inhibitor (such as the compound of Formula (I) or a pharmaceutically acceptable salt thereof) and a THR-β agonist (such as the compound of (II) or a pharmaceutically acceptable salt thereof) described herein. The kits may be used for any one or more of the uses described herein, and, accordingly, may contain instructions for the treatment as described herein.

Kits generally comprise suitable packaging. The kits may comprise one or more containers comprising any compound described herein or a pharmaceutically acceptable salt thereof. Each component can be packaged in separate containers or some components can be combined in one container where cross-reactivity and shelf life permit. In some embodiments, the kit includes a container comprising the SSAO inhibitor (such as the compound of Formula (I) or a pharmaceutically acceptable salt thereof) and the THR-β agonist (such as the compound of (II) or a pharmaceutically acceptable salt thereof). In other embodiments, the kit includes a first container comprising SSAO inhibitor (such as the compound of Formula (I) or a pharmaceutically acceptable salt thereof) and a second container comprising the THR-β agonist (such as the compound of (II) or a pharmaceutically acceptable salt thereof).

The kits may be in unit dosage forms, bulk packages (e.g., multi-dose packages) or sub-unit doses. For example, kits may be provided that contain sufficient dosages of a compound as disclosed herein, or a pharmaceutically acceptable salt thereof, and/or an additional pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to an individual.

### EXAMPLES

The combination treatment provided herein can be tested by administering the combination of the agents to a well-known mouse model and evaluating the results. Methods of such testing can be adapted from those known. See, e.g., US Pat. Pub. No. 2015/0342943, incorporated herein by reference.

### Example 1: Compound 1 Tolerability and Pharmacokinetics in Healthy Human Subjects

### Background

Semicarbazide-sensitive amine oxidase (SSAO) contributes to non-alcoholic steatohepatitis (NASH) by increasing oxidative stress through deamination of primary amines (e.g., methylamine, MMA) to aldehyde, ammonium, and H₂O₂ and by recruitment of inflammatory cells to the liver, exacerbating hepatic inflammation and injury. SSAO levels are elevated in NASH and correlate with fibrosis stage. Compound 1 is a selective, covalent SSAO inhibitor that decreases liver inflammation and fibrosis in a rat model of NASH. A single-ascending dose clinical trial of Compound 1 was performed.

The compounds described herein may be obtained by the methods described in WO 2018/028517, which is incorporated herein by reference in its entirety and specifically with respect to the methods of making the compounds detailed herein.

### Methods

Four groups of 8 healthy participants were randomized to receive Compound 1 (tosylate salt) capsule or matching placebo in a 3:1 ratio. Plasma levels of Compound 1 and PD biomarkers were determined at pre-dose and various time points post-dose. SSAO inhibition was determined by measuring relative reductions in plasma H₂O₂ generation after addition of an exogenous substrate (benzylamine). Endogenous methylamine (MMA) levels, predicted to increase upon SSAO inhibition, were measured in plasma. Safety was assessed for 7 (±3) days after dosing.

Plasma samples for Compound 1 concentration and SSAO activity determination were collected at 0.25, 0.5, 1, 2, 3, 4, 6, 8, 10, 12, 24, 48 (SSAO activity only), and 168 (SSAO activity only) hours after administration of a single dose of study medication (placebo or compound). Plasma PK parameters were determined by non-compartmental analysis. SSAO activity was assessed by measuring hydrogen peroxide (H₂O₂) generation levels in plasma samples from placebo and active Compound 1 recipients. Percent change in total amine oxidase activity was determined relative to the corresponding pre-dose (baseline) samples.

SSAO-specific amine oxidase levels in plasma were determined using a kinetic-based assay essentially as described previously (Schilter et al). Endogenous monoamine oxidases A and B were inhibited by adding pargyline to plasma samples prior to measuring H₂O₂ generation levels in placebo and active recipients. Maximum inhibition was defined by pre-dose (baseline) samples additionally treated with a high dose of Compound 1 and percent changes in SSAO-specific activity were calculated relative to baseline samples.

Results

32 healthy human participants (100% male, 63% Black, 19% Asian, 13% Caucasian) were enrolled and received a single oral dose of Compound 1 (1, 3, 6, and 10 mg, n=6 each) or placebo (n=2). Compound 1 plasma PK exposure increased in a greater than dose proportional manner between the 1 and 10 mg dose levels. The mean half-life of Compound 1 ranged from 1-3 hours. At 4 hours post-dose, near complete inhibition of plasma SSAO activity was seen in all dose cohorts and continued suppression was detected for up to 1 week after a single dose of Compound 1. Maximum plasma MMA levels increased with Compound 1. No clinically relevant adverse events or laboratory abnormalities were reported.

As shown in Table 1, doses 1, 3, 6, and 10 mg of Compound 1 were all well tolerated.

TABLE 1
Compound 1 Treatment Associated Adverse Events

| | 1 mg of a | 3 mg of a | 6 mg of a | | 10 mg of a | |
|---|---|---|---|---|---|---|
| | tosylate salt of | tosylate salt of | tosylate salt of | | tosylate salt of | |
| | Compound 1 | Compound 1 | Compound 1 | | Compound 1 | |
| | or placebo | or placebo | or placebo | | or placebo | All |
| | (n = 8) | (n = 8) | (n = 8) | | (n = 8) | (n = 32) |
| Subject | 0 | 0 | 2 | (25) | 3 (37.5) | 5 (15.6) |
| incidence of | | | | | | |
| any TEAE | | | | | | |
| Subject | 0 | 0 | 0 | | 1 (12.5) | 1 (3.1) |
| incidence of | | | | | | |
| TEAEs | | | | | | |
| considered | | | | | | |
| possibly | | | | | | |
| treatment- | | | | | | |
| related | | | | | | |
| TEAE | | | | | | |
| diagnosis and | | | | | | |
| frequency | | | | | | |
| constipation | 0 | 0 | 0 | | 1 (12.5) | 1 (3.1) |
| contact | 0 | 0 | 2 | (25) | 0 | 2 (6.3) |
| dermatitis | | | | | | |
| dysgeusia | 0 | 0 | 0 | | 1 (12.5) | 1 (3.1) |
| headache | 0 | 0 | 0 | | 1 (12.5) | 1 (3.1) |
| oral herpes | 0 | 0 | 0 | | 1 (12.5) | 1 (3.1) |
| sore throat | 0 | 0 | 1 | (12.5) | 0 | 1 (3.1) |

| | | | | | |
|---|---|---|---|---|---|
| upper | 0 | 0 | 0 | 1 (12.5) | 1 (3.1) |
| respiratory tract | | | | | |
| infection | | | | | |

Single doses of the tosylate salt of Compound 1 for SSAO were rapidly cleared from plasma and exhibited greater than dose proportional plasma PK between 1 and 10 mg.

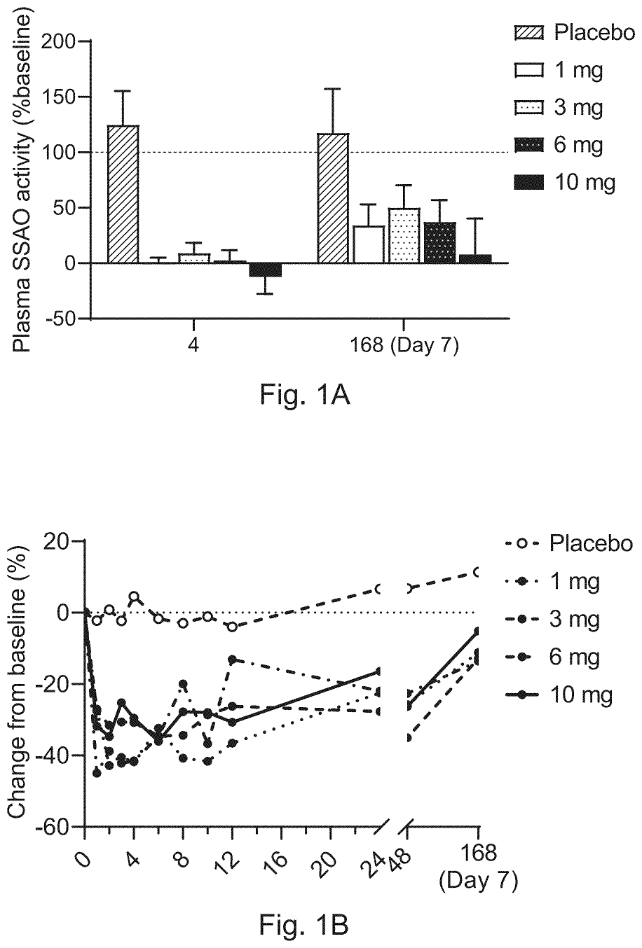

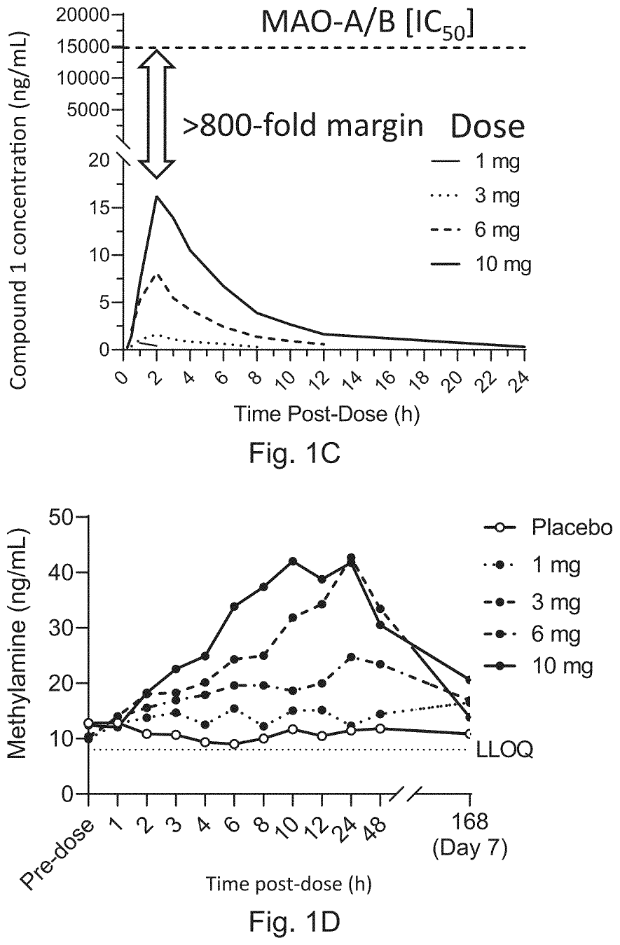

Single doses of Compound 1 rapidly and potently decreased plasma amine oxidase activity in all subjects as shown in FIG. 1A and FIG. 1B. Near complete inhibition of SSAO-specific activity as observed at 4 hours post dose (FIG. 1A and FIG. 1B). Inhibition of plasma SSAO amine oxidase activity and dose-dependent increases in plasma MMA were sustained up to 1 week after single doses of Compound 1, suggesting potent, covalent target engagement and supporting once daily dosing despite a short plasma half-life (FIG. 1A and FIG. 1B).

The concentrations (Cₘₐₓ) of Compound 1 or placebo were more than 800 times lower than the IC₅₀ concentrations for MAO-A and MAO-B at all dose levels (FIG. 1C).

TABLE 2
Biochemical activity (IC₅₀ μM)

| SSAO inhibitor | SSAO | MAO-A | MAO-B |
|---|---|---|---|
| Compound 1 | 0.0065 | >50 | >50 |
| BI 1467335 (PXS-4728A) | 0.005 | >100 | 2.7 |

Dose-dependent increases in methylamine were observed, indicating potent plasma SSAO target engagement across the dose range. FIG. 1D.

### Conclusions

Compound 1 was safe and well tolerated in healthy subjects administered a single oral dose ranging from 1 mg to 10 mg. Compound 1 inhibited SSAO activity for up to seven days after a single dose. This suggests that Compound 1 may be effective for treating liver diseases or disorders by selectively inhibiting SSAO. It may also exhibit SSAO activity for seven days after only a single dose, suggesting that daily administration for one week may exert a therapeutic effect for a two-week period.

### Example 2: Multiple Ascending Dose Trial of Compound 1 in Healthy Human Subjects

A multiple-ascending dose clinical trial of Compound 1 was performed. 3 groups of 8 healthy participants were randomized to receive multiple once daily (QD) doses of Compound 1 or matching placebo in a 3:1 ratio for 7 days (1 mg and 4 mg) or 14 days (10 mg). Plasma levels of Compound 1 and PD biomarkers (plasma amine oxidase activity and methylamine levels) were determined at pre-dose and various timepoints post-dose. Safety, including laboratory, vital signs, and ECG, among others, was assessed for up to 14 days after last dose with no notable findings across subjects. All adverse events were considered mild (grade 1), except for one moderate (grade 2) adverse event in the placebo treatment group. No subject discontinued due to an adverse event (see Table 3).

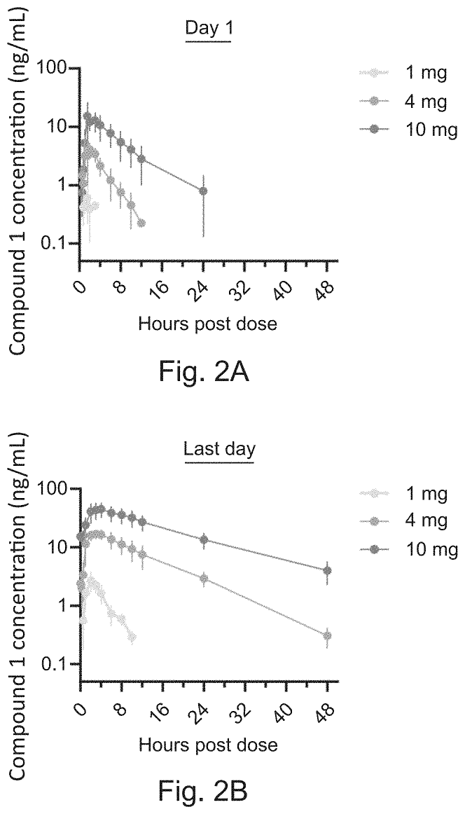

Compound 1 plasma PK exposure increases were greater than dose proportional between dose groups on Day 1, and significant accumulation at each dose level was observed after multiple QD doses. The accumulation ratio between the first and last day of dosing decreased as dose increased. Steady state was achieved in the highest dose cohort (10 mg) after 7 days. Compound 1 half-life increased with dose, consistent with a saturable target-mediated clearance (see FIG. 2A and FIG. 2B, Table 4).

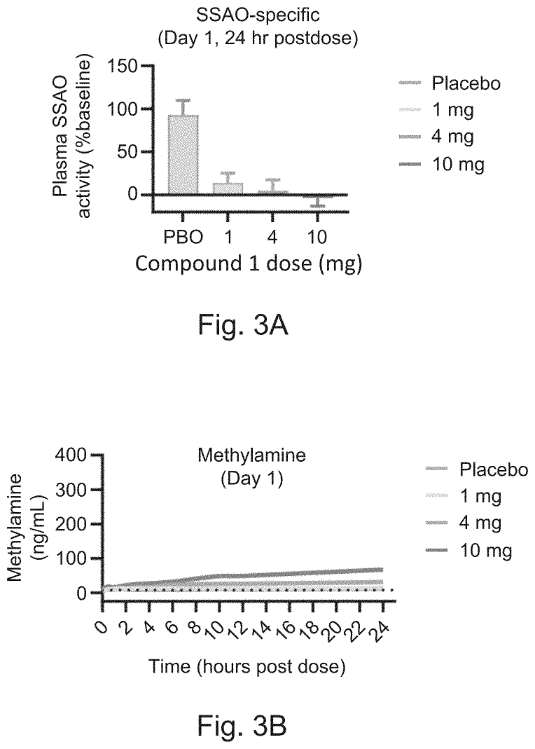

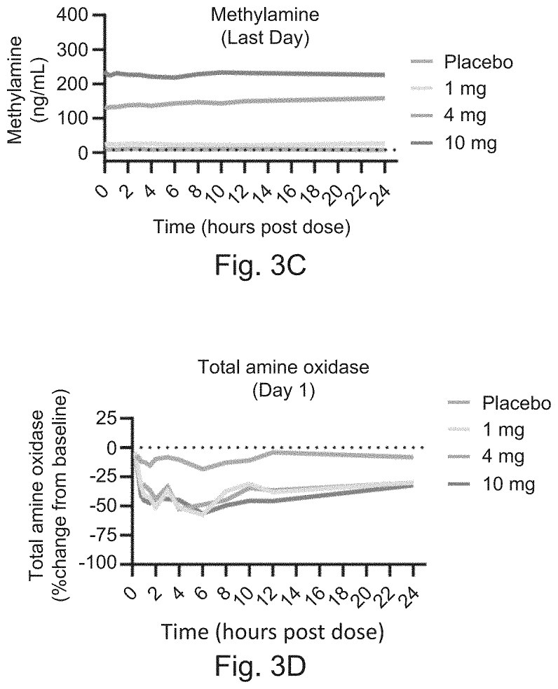

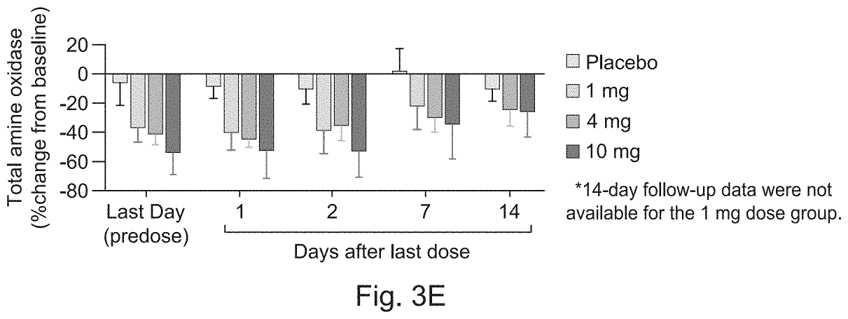

Near complete inhibition of plasma SSAO activity was seen on Day 1 in all dose cohorts (FIG. 3A), resulting in an increase in methylamine (FIG. 3B), which is an endogenous substrate of SSAO. Plasma methylamine levels increased in a dose proportional manner (FIG. 3B). After multiple doses, further increases in plasma methylamine were observed on the last day of Compound 1 administration (FIG. 3C). Inhibition of total amine oxidase was incomplete due to the presence of plasma amine oxidases that were not inhibited by Compound 1 (e.g., MAO-A, MAO-B) (FIG. 3D, E)

Compound 1 was safe and well tolerated in healthy subjects when administered up to 10 mg QD for 14 days. Steady state levels of Compound 1 were achieved after 7 days of dosing supporting a QD dosing regimen. Near complete inhibition of plasma SSAO amine oxidase activity and dose-dependent increases in plasma methylamine were sustained up to 2 weeks after cessation of dosing, suggesting that daily administration of Compound 1 for two weeks may exert a therapeutic effect for a two-week period after cessation of dosing.

TABLE 3
Compound 1 Treatment Associated Adverse Events

| | | 1 mg | 4 mg | 10 mg | Overall |
|---|---|---|---|---|---|
| | Placebo | Compound 1 | Compound 1 | Compound 1 | Compound 1 |
| | (n = 6) | (n = 6) | (n = 6) | (n = 6) | (n = 18) |
| Subject incidence of | 3 (50) | 2 (33.3) | 2 (33.3) | 6 (100) | 10 (55.6) |
| any TEAE, n (%) | | | | | |
| Subject incidence of | 1 (16.7) | 1 (16.7) | 0 | 0 | 1 (5.6) |
| TEAEs considered | | | | | |
| possibly treatment- | | | | | |
| related¹, n (%) | | | | | |

TEAE diagnosis and frequency

| | | | | | |
|---|---|---|---|---|---|
| Back pain | 1 (16.7) | 0 | 0 | 0 | 0 |
| Catheter site | 1 (16.7) | 0 | 0 | 0 | 0 |
| inflammation | | | | | |
| Contusion | 1 (16.7) | 0 | 0 | 0 | 0 |
| Dermatitis | 0 | 1 (16.7) | 2 (33.3) | 0 | 3 (16.7) |
| contact | | | | | |
| Diarrhea | 1 (16.7) | 0 | 0 | 0 | 0 |
| Dizziness | 0 | 1 (16.7) | 0 | 0 | 1 (5.6) |
| Headache | 0 | 1 (12.5) | 0 | 0 | 1 (5.6) |
| Medical device site | 2 (33.3) | 0 | 0 | 6 (100) | 6 (33.3) |
| reaction² | | | | | |
| Rhinitis | 0 | 0 | 0 | 1 (16.7) | 1 (5.6) |

¹One subject who received 1 mg Compound 1 for 7 days had an event (headache) considered possibly related to treatment.
²All 8 subjects (6 Compound 1, 2 placebo) in the 10 mg cohort had mild events of contact dermatitis at the site of ECG leads (“Medical device site reaction”); ECGs were at least daily, per protocol.

TABLE 4
Compound 1 Pharmacokinetic data

| Dose | | Cₘₐₓ | Tₘₐₓ | AUC₀₋ₜ | t_{1/2} | AR | AR |
|---|---|---|---|---|---|---|---|
| (mg) | Day | (ng/mL) | (h) | (h*ng/mL) | (h) | (Cₘₐₓ) | (AUC) |
| 1 | 1 | 0.568 (64) | 1.53 (0.82, | NC | | | |
| | | | 2.00) | | | | |
| | Last | 2.75 (36) | 2.51 (1.02, | 13.3 (14) | 2.27 (2.00, | 5.98 (49) | NC |
| | | | 3.00) | | 2.48) | | |
| 4 | 1 | 5.11 (64) | 2.02 (1.02, | 17.1 (29) | | | |
| | | | 3.07) | | | | |
| | Last | 18.6 (20) | 3.03 (1.03, | 198 (32) | 6.71 (2.85, | 4.32 (34) | 11.7 (22) |
| | | | 3.07) | | 8.13 | | |
| 10 | 1 | 17.7 (49) | 1.90 (1.48, | 102 (53) | | | |
| | | | 4.00) | | | | |
| | Last | 47.5 (31) | 3.98 (2.03, | 656 (29) | 13.3 (9.97, | 3.35 (57) | 8.2 (63) |
| | | | 4.02) | | 14.9) | | |

Tₘₐₓ and t_{1/2} presented as: median (min, max). All other PK parameters presented as: mean (CV %).
AR = accumulation ratio between Day 1 and Last Day.
NC = not calculable.

### Example 3: Activity and Pharmacokinetics of Compounds of Formula (II)

Exemplary compounds of formula (II) are provided in Table 5 below. Compound 2 is listed in the table as compound number 2. The compounds described herein are disclosed in US Application Publication No. 20200190064, the contents of which are incorporated by reference in their entirety, and specifically with respect to the compounds of formula (II), such as compound 2, or a pharmaceutically acceptable salt or enantiomer thereof, as well as methods of making and using the foregoing.

TABLE 5
Exemplary compound of formula (II)

| Compound | Structure |
|---|---|
| 2 | 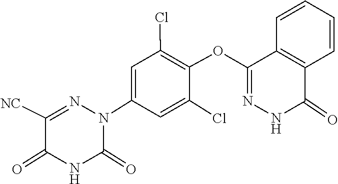 [SMILES: N#Cc1nn(-c2cc(Cl)c(Oc3n[nH]c(=O)c4ccccc34)c(Cl)c2)c(=O)[nH]c1=O] |
| 3 | 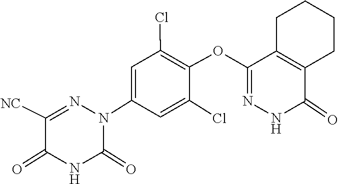 [SMILES: N#Cc1nn(-c2cc(Cl)c(Oc3n[nH]c(=O)c4c3CCCC4)c(Cl)c2)c(=O)[nH]c1=O] |
| 4 | 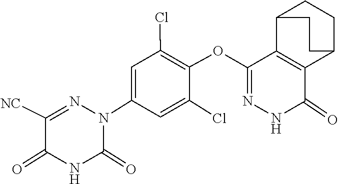 [SMILES: N#Cc1nn(-c2cc(Cl)c(Oc3n[nH]c(=O)c4c3C3CCC4CC3)c(Cl)c2)c(=O)[nH]c1=O] |
| 5 | 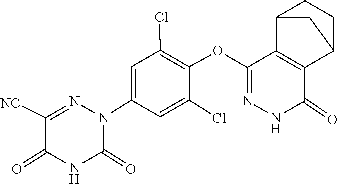 [SMILES: N#Cc1nn(-c2cc(Cl)c(Oc3n[nH]c(=O)c4c3C3CCC4C3)c(Cl)c2)c(=O)[nH]c1=O] |
| 6 | 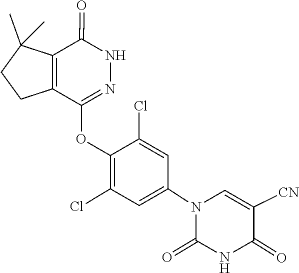 [SMILES: CC1(C)CCc2c(Oc3c(Cl)cc(-n4cc(C#N)c(=O)[nH]c4=O)cc3Cl)n[nH]c(=O)c21] |
| 7 | 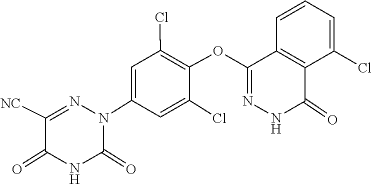 [SMILES: N#Cc1nn(-c2cc(Cl)c(Oc3n[nH]c(=O)c4c(Cl)cccc34)c(Cl)c2)c(=O)[nH]c1=O] |
| 8 | 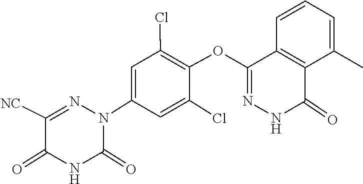 [SMILES: Cc1cccc2c(Oc3c(Cl)cc(-n4nc(C#N)c(=O)[nH]c4=O)cc3Cl)n[nH]c(=O)c12] |
| 9 | 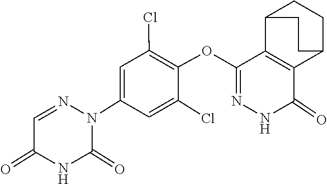 [SMILES: O=c1cnn(-c2cc(Cl)c(Oc3n[nH]c(=O)c4c3C3CCC4CC3)c(Cl)c2)c(=O)[nH]1] |
| 10 | 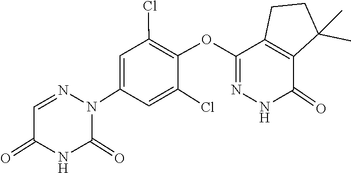 [SMILES: CC1(C)CCc2c(Oc3c(Cl)cc(-n4ncc(=O)[nH]c4=O)cc3Cl)n[nH]c(=O)c21] |
| 11 | 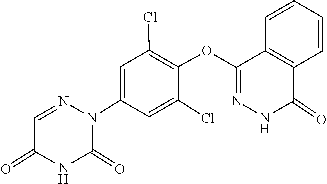 [SMILES: O=c1cnn(-c2cc(Cl)c(Oc3n[nH]c(=O)c4ccccc34)c(Cl)c2)c(=O)[nH]1] |

A compound of formula (II), in some embodiments, is selected from the group consisting of:
2-(3,5-dichloro-4-((4-oxo-3,4,5,6,7,8-hexahydrophthalazin-1-yl)oxy)phenyl)-3,5-dioxo-2,3,4,5-tetrahydro-1,2,4-triazine-6-nitrile;
2-(3,5-dichloro-4-((4-oxo-3,4,5,6,7,8-hexahydro-5,8-ethanophthalazin-1-yl)oxy)phenyl)-3,5-dioxo-2,3,4,5-tetrahydro-1,2,4-triazine-6-nitrile;
2-(3,5-dichloro-4-((4-oxo-3,4,5,6,7,8-hexahydro-5,8-methanophthalazin-1-yl)oxo)phenyl)-3,5-dioxo-2,3,4,5-tetrahydro-1,2,4-triazine-6-nitrile;
1-(3,5-dichloro-4-((7,7-dimethyl-1-oxo-2,5,6,7-tetrahydro-1H-cyclopentane[d]pyridazin-4-yl)oxy)phenyl)-2,4-dioxo-1,2,3,4-tetrahydropyrimidine-5-nitrile;
2-(3,5-dichloro-4-((4-oxo-3,4-dihydrophthalazin-1-yl)oxo)phenyl)-3,5-dioxo-2,3,4,5-tetrahydro-1,2,4-triazine-6-nitrile;
2-(3,5-dichloro-4-((5-chloro-4-oxo-3,4-dihydrophthalazin-1-yl)oxo)phenyl)-3,5-dioxo-2,3,4,5-tetrahydro-1,2,4-triazine-6-nitrile;
2-(3,5-dichloro-4-((5-methyl-4-oxo-3,4-dihydrophthalazin-1-yl)oxo)phenyl)-3,5-dioxo-2,3,4,5-tetrahydro-1,2,4-triazine-6-nitrile;
2-(3,5-dichloro-4-((4-oxo-3,4,5,6,7,8-hexahydro-5,8-ethanophthalazin-1-yl)oxo)phenyl)-1,2,4-triazine-3,5(2H,4H)-dione;
2-(3,5-dichloro-4-((7,7-dimethyl-1-oxo-2,5,6,7-tetrahydro-1H-cyclopentyl[d]pyridazin-4-yl)oxo)phenyl)-1,2,4-triazine-3,5(2H,4H)-dione; and
2-(3,5-dichloro-4-((4-oxo-3,4-dihydrophthalazin-1-yl)oxo)phenyl)-1,2,4-triazine-3,5-(2H,4H)dione.

A compound of formula (II) has a good agonistic activity toward the THR-β receptor, and an improved selectivity toward THRα as compared with Reference compound in the reference documents (“Discovery of 2-[3,5-Dichloro-4-(5-isopropyl-6-oxo-1,6-dihydropyridazin-3-yloxy)phenyl]-3,5-dioxo-2,3,4,5-tetrahydro[1,2,4]triazine-6-carbonitrile (MGL-3196), a Highly Selective Thyroid Hormone Receptor β Agonist in Clinical Trials for the Treatment of Dyslipidemia,” Martha et al., Journal of Medicinal Chemistry, 2014, 3912-3923). The structure of the reference compound is

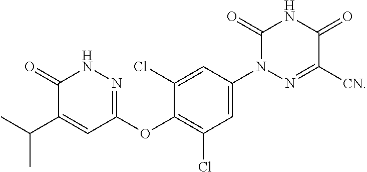 [SMILES: CC(C)c1cc(Oc2c(Cl)cc(-n3nc(C#N)c(=O)[nH]c3=O)cc2Cl)n[nH]c1=O]

Test data are shown in Table 6 and Table 7.

TABLE 6
Binding activity of compounds of formula
(II) to the thyroxine receptor beta

| | IC50 | | |
|---|---|---|---|
| | THR-β | THR-α | THR-α/-β |
| | binding force | binding force | selectivity |
| Compound | (μM) | (μM) | (factor) |
| 2 | 0.17 | >10 | >58.8 |
| 3 | 1.23 | >10 | >8.1 |
| 4 | 2.33 | >10 | >4.29 |
| 5 | 5.2 | >10 | >1.92 |
| 6 | 0.36 | 4.3 | >11.9 |
| 7 | 1.47 | >10 | >6.80 |
| 8 | 1.78 | >10 | 5.61 |
| 9 | 0.80 | 0.2 | 0.25 |
| 10 | 0.17 | 1.22 | 7.17 |
| 11 | 0.262 | | |
| Reference compound | 0.26 | 5.0 | 19.2 |
| triiodothyronine (T3) | 0.00052 | 0.00026 | — |

TABLE 7
Agonistic activity of compounds of formula
(II) toward the thyroxine receptor beta

EC₅₀

| | THR-β | THR-α |
|---|---|---|
| | agonistic activity | agonistic activity |
| Compound | (μM) | (μM) |
| 2 | 1.75 | 3.98 |
| 6 | 2.45 | 4.25 |
| 9 | 0.79 | 1.08 |
| 10 | 0.097 | 0.123 |
| Reference Compound | 2.48 | 4.57 |
| triiodothyronine (T3) | 0.001 | 0.0005 |

Compared with the reference compounds, exemplary compounds of formula (II) showed higher THR-β activity (<0.2 μM), and/or higher selectivity to THRα. The data also suggested that the compound of formula (II) can activate the downstream signal of the thyroid hormone receptor beta.

Pharmacokinetic Evaluation: Six healthy male SD rats, commercially available from Shanghai Sippr-Bk Laboratory Animal Co., Ltd., with an animal production license No.: SCxK(Shanghai) 2008-0016, were divided into 2 groups, 3 in each group.

Drug Preparation: a certain amount of the drug was taken and added into a 2% Klucel LF+0.1% Tween 80 aqueous solution, to prepare a clear solution or a uniform suspension.

Dosage: SD rats were fasted overnight and given the drug by intragastric infusion at an administrated dose of 2 mg/kg and an administrated volume of 10 mL/kg each.

Operation: rats were dosed by intragastric infusion with the compounds. At least 0.2 mL of blood was collected from the vena caudalis at 15 min, 30 min, 1 h, 2 h, 4 h, 6 h, 10 h, and 24 h before and after the dosage; the blood was then placed in heparinized sample tubes, centrifuged at 4° C. and 3500 rpm for 10 min to separate the plasma. The heparinized sample tubes were then stored at −20° C., and the rats were allowed to eat food 2 h after the dosage.

Determination of contents of the compounds to be tested in the plasma of rats after intragastric infusion of the drugs at different concentrations: the plasma samples were thawed at room temperature, 50 μL each was taken and added into 130 μL of an internal standard working solution (1000 ng/mL, acetonitrile, tolbutamide), and the mixture was whirled for about 1 min and then centrifugated at 4° C. and 13000 rpm for 10 min. 50 μL of the supernatant was taken and mixed with 100 μL of 50% acetonitrile water, and then introduced for LC/MS/MS analysis.

Results of the pharmacokinetic parameters are shown in Table 8.

TABLE 8
Pharmaceutical metabolism data of rats

| | | | Peak blood drug | | |
|---|---|---|---|---|---|
| | Dose | Time to peak | concentration | Curve area | Half-life |
| Compound | (mg/kg) | (h) | (ng/mL) | (ng · h/mL) | (h) |
| 2 | 2.0 | 4.67 ± 1.15 | 2007 ± 106 | 24790 ± 3704 | 4.56 ± 0.42 |
| 6 | 2.0 | 5.33 ± 1.15 | 727 ± 183 | 9242 ± 1245 | 5.14 ± 0.83 |
| Reference | 2.0 | 5.3 ± 1.15 | 1163 ± 97.1 | 12854 ± 961 | 3.53 ± 0.42 |
| Compound | | | | | |

The data showed that exemplary compounds demonstrated good pharmacokinetic absorption and significant pharmacokinetic advantages. Compared with the reference compound, exemplary compounds showed higher Cmax values and exposure amounts at the same dose and preparation.

### Example 4: Effects of Compound 2 on Serum Cholesterol and Triglycerides

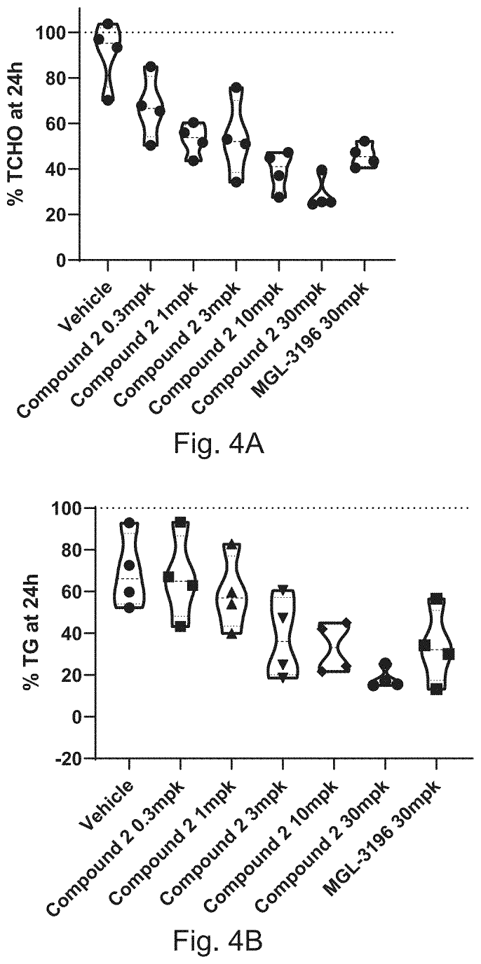

SD rats were fed a high cholesterol diet for 2 weeks, increasing the serum cholesterol levels ˜4-fold over that time. Single doses of Compound 2 from 0.3 to 30 mpk or a single 30 mpk dose of MGL-3196 were injected IP and serum was analyzed for total serum cholesterol and triglycerides 24 h after the injection. Total cholesterol in the serum was significantly reduced from 30-70% with Compound 2 (FIG. 4A). Compound 2 significantly reduced serum triglycerides from 30-80% from time 0 (FIG. 4B).

### Example 5: Effects of Compound 2 on Mouse NASH Model

C57BL/6J mice were fed a high fat diet for 10 weeks to induce obesity (>38 g BW). Obese mice were injected intraperitoneally (i.p.) twice a week for four weeks with 0.5 μl/g 25% CCl₄ (formulated in olive oil) to induce fibrosis, and one group of normal BW mice were injected i.p. twice a week for four weeks with olive oil to serve as a healthy control. During the same dosing period, obese mice were fed orally once a day for 28 days with vehicle or varying doses of Compound 2. On CCl₄ dosing days, CCl₄ was administered at 4 hours post compound or vehicle dosing. On day 27, all animals were fasted for about 16 hours before terminal euthanasia. On day 28, all animals were sacrificed and various biological parameters were analyzed. Total body, liver, heart and brain weight were measured and changes in liver and heart weight were normalized using brain weight.

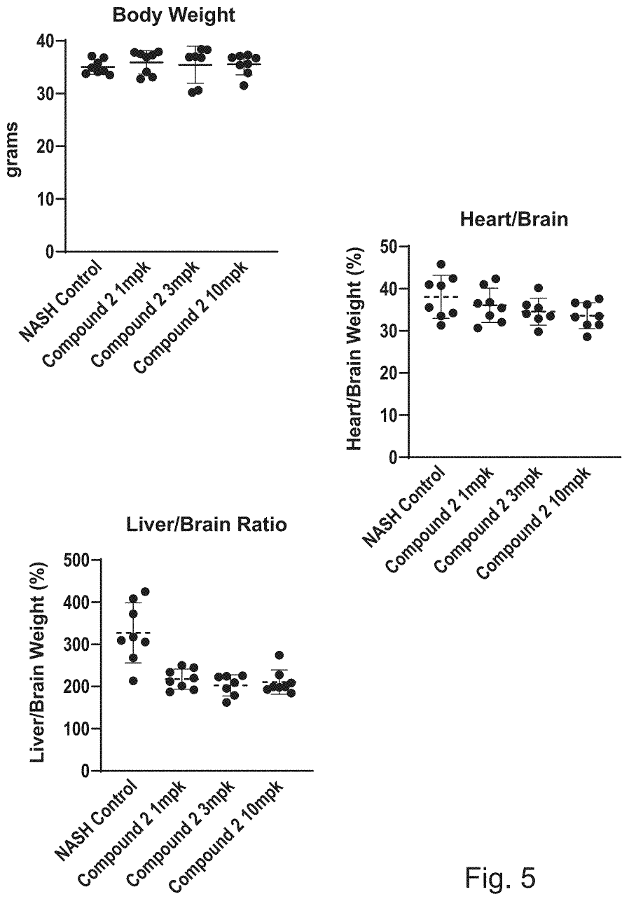

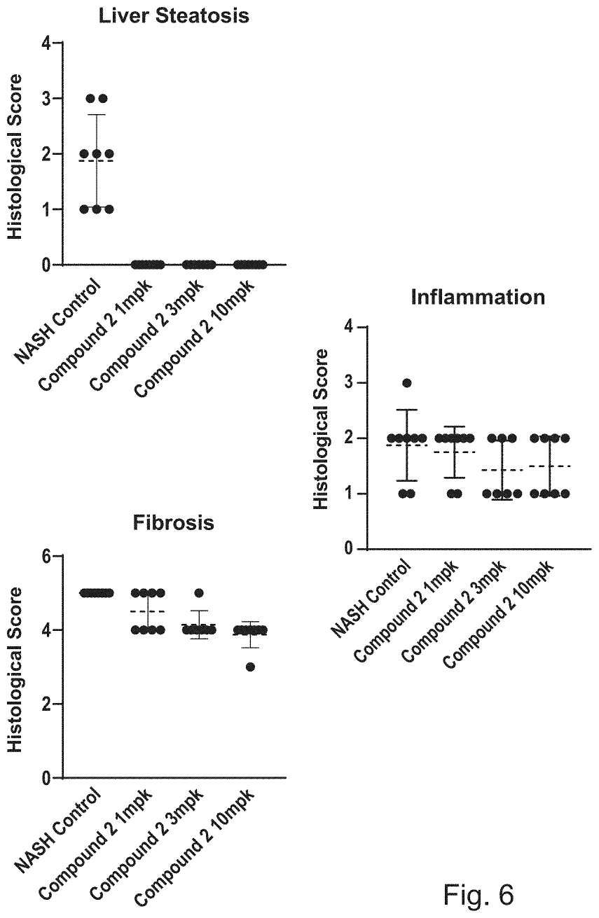

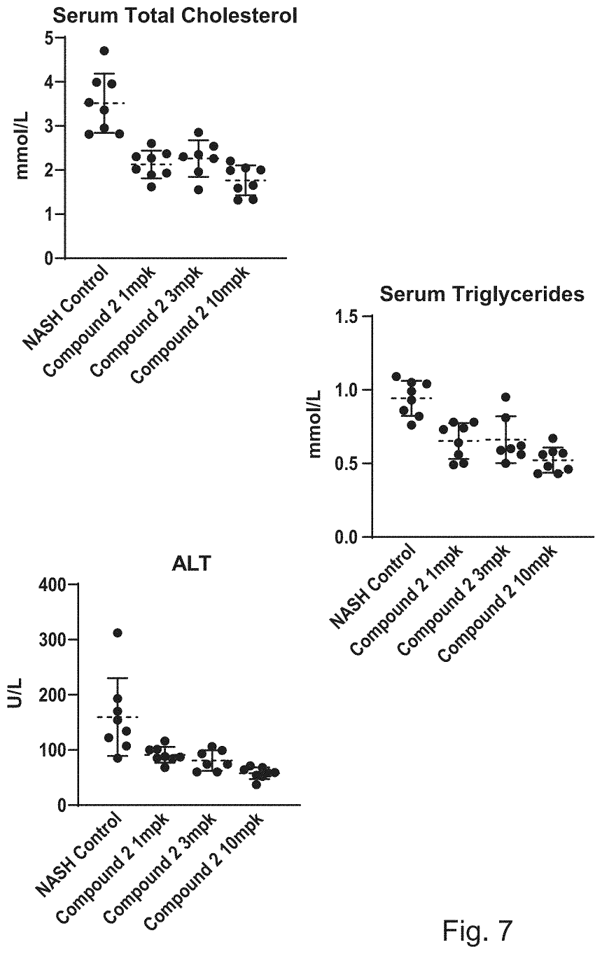

Compound 2 significantly reduced liver/brain weight with no effect on total body weight or heart/brain weight (FIG. 5). Liver tissue histology was analyzed for effects of Compound 2 on steatosis, inflammation and fibrosis. Compound 2 significantly reduced steatosis at all doses tested, showed a trend in inflammation reduction and significantly reduced liver fibrosis at 3 and 10 mpk (FIG. 6). Compound 2 also significantly reduced serum total cholesterol, triglycerides and ALT at all doses tested (FIG. 7).

Liver samples were collected for whole transcriptome analysis by RNA sequencing (RNAseq). RNAseq library (n=5 per group) preparation and sequencing was performed using Illumina standard protocols. Alignment of sequencing reads was performed using STAR aligner software and read counts were estimated using RSEM. Differentially expressed genes (compared to vehicle-treated NASH control mice) were determined using EdgeR software. Gene ontology analysis was performed using Advaita software with fold-change and adjusted p-value cutoffs of >1.5 and <0.05, respectively. Gene ontologies were derived from the Gene Ontology Consortium database (2019 Apr. 26) (Ashburner et al., Gene ontology: Tool for the unification of biology. Nature Genetics 25(1): 25-9 (2000); Gene Ontology Consortium, Creating the Gene Ontology Resource: Design and Implementation. Genome Research 11: 1425-1433 (2001)). Compound 2 had a significant effect on expression of genes associated with collagen extracellular matrix and hepatic stellate cell activation, primarily by reducing their expression levels relative to NASH control mice (FIG. 8).

### Example 6: pH and Solubilizer Effect on Compound 2 Solubility

Solubility of compound 2 (potassium salt) was evaluated at various pH levels. The solubility of compound 2 in aqueous solution was pH dependent and increased with pH, as shown in Table 9. In the presence of a solubilizer (sodium lauryl sulfate, SLS), the solubility of compound 2 further improved to 308 μg/mL in pH 10.0 buffer+2 wt % SLS at 25° C. after 24 h.

TABLE 9
pH and solubilizer effect on solubility of compound 2

| | Solubility (μg/mL) | |
|---|---|---|
| Solvent | 24 h (without 2 wt % SLS) | 24 h (with 2 wt % SLS) |
| 0.1N HCl | <0.03 | 5.7 |
| pH 2.0 buffer | <0.07 | 5.5 |
| pH 4.0 buffer | <0.07 | 10.0 |
| pH 6.0 buffer | 2.2 | 42.3 |
| pH 8.0 buffer | 10.8 | 301.9 |
| pH 10.0 buffer | 10.2 | 308.2 |

Approximate pKa of compound 2 = 4.12.

### Example 7: Compound 2 Formulations, Pharmacokinetics, Food Effect in Beagle Dogs

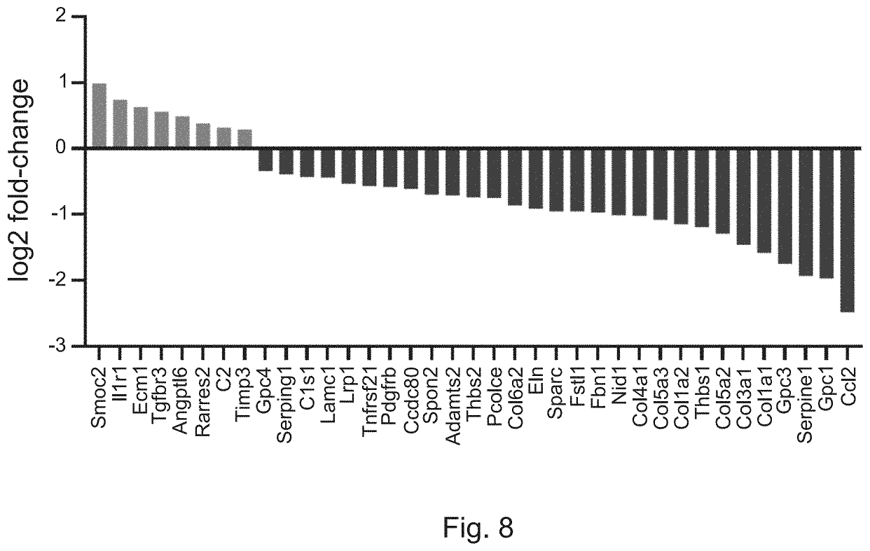

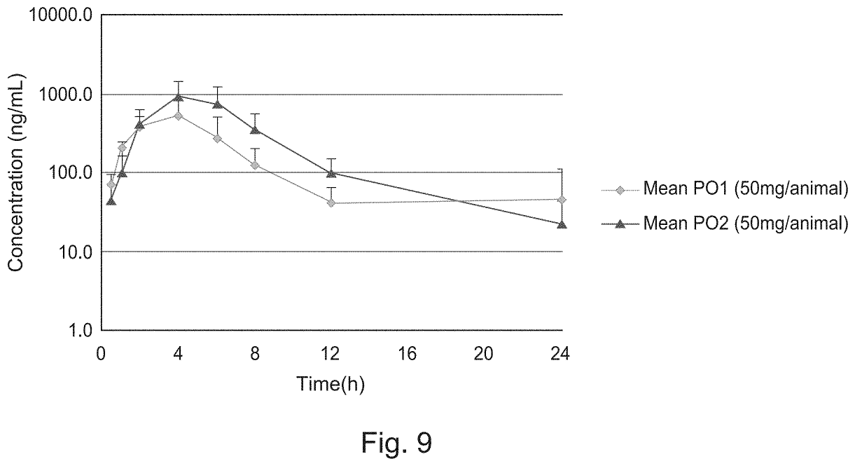

To determine the effect of the solubilizer on the uptake of compound 2, 50 mg (based on free acid) Compound 2 was formulated in a capsule with or without 5 wt % SLS (see Table 10) and was administered to fasted beagle dogs pre-treated with pentagastrin (6 μg/kg, administered by intramuscular injection 30±2 minutes prior to administration of compound 2). The plasma concentration of compound 2 was measured over 24 hours (see FIG. 9). Formulation with 5 wt % SLS increased the exposure (Cₘₐₓ, AUC) of compound 2 by more than 70% (see Table 11).

TABLE 10
Composition of 50 mg compound 2 capsule formulations.

| | Formulation 1 | Formulation 2 |
|---|---|---|
| | (PO1) | (PO2) |
| | (wt %) | (wt %) |
| Compound 2 | 18.25 | 18.25 |
| Microcrystaltine Cellulose PH-102 | 51.16 | 47.83 |
| Mannitol 200SD | 25.58 | 23.92 |
| Sodium Lauryl Sulfate SLS Fine | 0 | 5.0 |
| Croscarmellose Sodium | 3.0 | 3.0 |
| Colloidal Silica Dioxide Aerosil | 1.0 | 1.0 |
| 200 Pharma | | |
| Magnesium Stearate LIGAMED | 1.0 | 1.0 |
| MF-2-V | | |

TABLE 11
Pharmacokinetics of compound 2 after dosing
50 mg capsules in beagle dogs (n = 5).

| | Formulation 1 | Formulation 2 |
|---|---|---|
| Cₘₐₓ (ng/mL) | 572 | 1010 |
| Tₘₐₓ (h) | 3.60 | 3.20 |
| t_{1/2} (h) | 4.46 | 4.11 |
| AUC₀₋ₗₐₛₜ (ng*h/mL) | 3420 | 6210 |
| AUC_{0-inf} (ng*h/mL) | 3700 | 6360 |

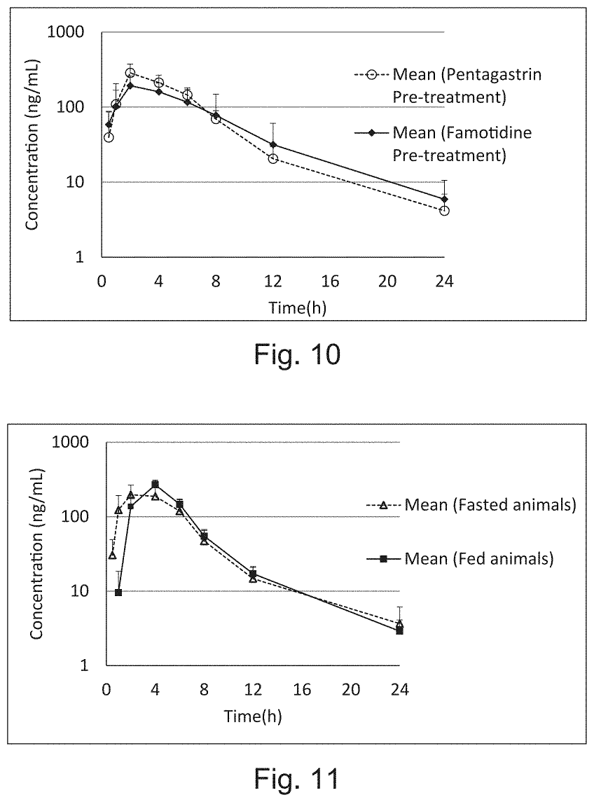

To determine the pH effect on PK performance, beagle dogs were divided into two groups (n=3 per group). For group 1, dogs were pre-treated with pentagastrin (6 μg/kg, intramuscular injection 30±2 minutes prior to administration of compound 2). For group 2, dogs were pre-treated with famotidine (2 tablets, 20 mg/tablet, oral administration 180±10 minutes prior to administration of compound 2). Compound 2 (10 mg capsule, 5% SLS) was administered, and plasma concentrations of compound 2 were monitored for 24 h and are depicted in FIG. 10. Minimal pH effect occurred in dogs under the study conditions.
To determine the food effect on PK performance, beagle dogs were divided into two groups (n=3 per group). For the fasted group, dogs were fasted overnight through 4-hours post dosing. For the fed group, dogs were fed high fat food 30 minutes prior to administration. Plasma concentrations of compound 2 were monitored for 24 h. Compound 2 (10 mg capsule, 5% SLS) was administered, and plasma concentrations of compound 2 were monitored for 24 h and are depicted in FIG. 11. Food intake delayed Compound 2 Tₘₐₓ but did not have an impact on the plasma exposure.

### Example 8: Single Ascending Dose Trial of Compound 2 in Healthy Human Subjects

### Methods

A single-ascending dose clinical trial of Compound 2 (potassium salt) was performed. Four groups of 8 healthy participants were randomized to receive Compound 2 (3 mg, 10 mg, 30 mg, or 60 mg capsule) or matching placebo in a 3:1 ratio (n=6 active and n=2 placebo) and were administered during the fasted state on Day 1 of the study. Plasma levels of Compound 2 and PD biomarkers were determined at pre-dose and various time points post-dose.

Adverse event (AE) monitoring, routine clinical laboratory testing (including thyroid axis testing [free and total thyroid hormone triiodothyronine (T3), free and total thyroid hormone thyroxine (T4), thyroid stimulating hormone (TSH)] cardiac biomarkers [CK-MB, troponin I], and liver biochemistry), intensive vital signs, cardiac telemetry, and electrocardiograms were assessed throughout the study. Compound 2 plasma and urine concentrations were determined using validated liquid chromatography-tandem mass spectrometry assay

Plasma samples for Compound 2 concentration and PK sampling were collected at pre-dose and at 0.5, 1, 2, 3, 4, 6, 8, 12, 24, 48, and 72 hours after administration of a single dose of study medication (placebo or compound). Urine samples for Compound 2 concentration and PK sampling were collected pre-dose and at the following timepoints: 0-6 hours, 6-12 hours, 12-24 hours, and 24-48 hours. PK parameters were estimated via noncompartmental methods using Phoenix® WinNonlin® (Certara, LP, Princeton, N.J.). Concentrations of serum pharmacodynamic (PD) biomarkers apolipoprotein B (Apo B) and sex hormone binding globulin (SHBG) were measured using an immunoassay and serum lipids were determined using spectrophotometry.

PD sampling was performed pre-dose, and 48 hours and 72 hours post-dose. Percent change from baseline for PD markers were calculated using an ANCOVA model with percent change from baseline as dependent variable, treatment group as fixed effect, and baseline as covariate. Analyses used observed data only without imputation for missing data.

### Results

### Treatment-Emergent Adverse Events Monitoring

All adverse events were mild or moderate in severity and largely unrelated to the study drug. No cardiac related AEs (e.g., tachycardia, arrythmias) were reported, and no remarkable changes in vital signs or ECG parameters were seen (see Table 12)

TABLE 12

| | | 3 mg | 10 mg | 30 mg | 60 mg |
|---|---|---|---|---|---|
| | Placebo | Cmpd 2 | Cmpd 2 | Cmpd 2 | Compd 2 |
| | (n = 8) | (n = 6) | (n = 6) | (n = 6) | (n = 6) |
| Any AE, all CTCAE | 1 (12.5%) | 2 (33.3%) | 0 | 2 (33.3%) | 0 |
| grades | | | | | |
| CTCAE Grade 1 | 0 | 0 | 0 | 1 (16.7%) | 0 |
| CTCAE Grade 2 | 0 | 0 | 0 | 1 (16.7%) | 0 |
| CTCAE Grade 3 or | 0 | 0 | 0 | 0 | 0 |
| higher | | | | | |
| Serious AEs | 0 | 0 | 0 | 0 | 0 |

AEs by relationship to drug

| | | | | | |
|---|---|---|---|---|---|
| Not related | 1 (12.5%) | 2 (33.3%) | 0 | 1 (16.7%) | 0 |
| Unlikely related | 0 | 0 | 0 | 0 | 0 |
| Possibly related | 0 | 0 | 0 | 1 (16.7%)* | 0 |
| Related | 0 | 0 | 0 | 0 | 0 |

*One subject reported headache on Day 1 and Day 3 which resolved spontaneously, and pleuritic pain (left axilla) on Day 2 which lasted a few hours and resolved after one dose (1000 mg) of acetaminophen.

### Safety and Thyroid Axis Monitoring

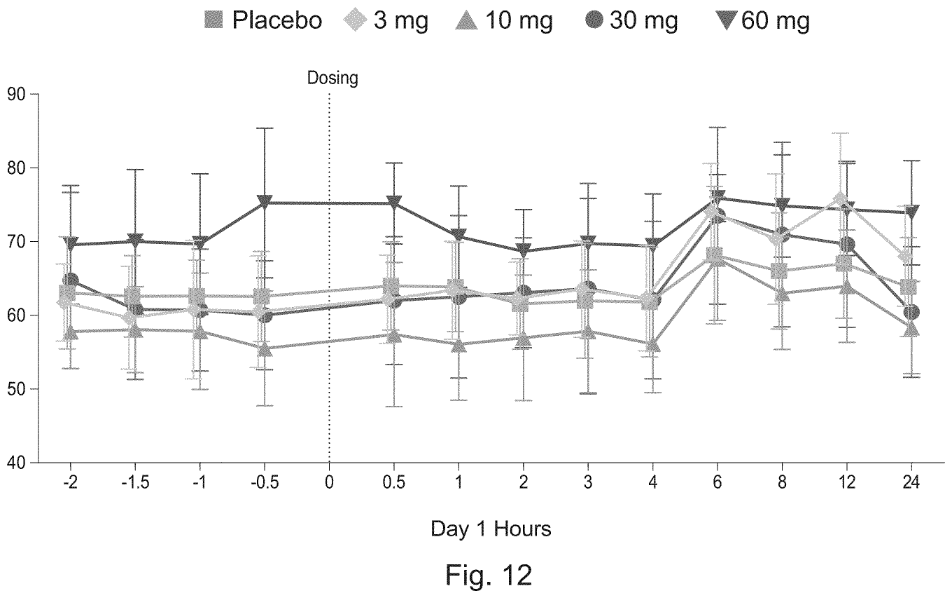

Heart rates remained stable and within the normal range for 24 hours after dosing with Compound 2, following a similar pattern as seen in the placebo group (FIG. 12). TSH, free T3, and free T4 levels remained within the normal range. No subjects experienced increases in ALT more than 2-times greater than the upper limit of normal (ULN); no subjects exhibited bilirubin levels above the normal range. In addition, no notable changes in cardiac biomarkers (troponin I, CK-MB) or other clinical safety tests were observed.

### Pharmacokinetics

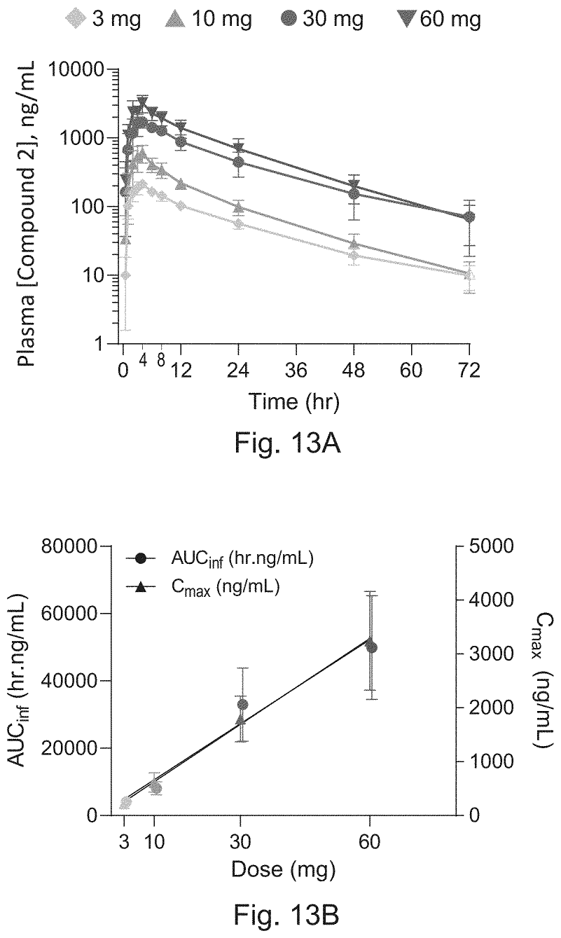

Compound 2 was absorbed with low variability (% CV≤33%) under fasted conditions. Exposures (AUC, Cₘₐₓ) were approximately dose-proportional. Median half-life of compound 2 ranged from 13.8 to 17.3 h, supporting once daily dosing. Minimal renal excretion was determined at all doses. See Table 13, FIG. 13A, FIG. 13B.

TABLE 13

| Dose of | AUC_{inf} | | Cₘₐₓ | | Tₘₐₓ | t_{1/2} | fₑ |
|---|---|---|---|---|---|---|---|
| Compound 2 | (hr*ng/mL) | | (ng/mL) | | (h) | (h) | (% dose, urine) |
| 3 mg (n = 6) | 4060 | (16.6) | 217 | (13.9) | 4.00 (3.00-4.00) | 17.3 (13.8-20.1) | 2.95 (62) |
| 10 mg (n = 6) | 8110 | (23.9) | 614 | (29.0) | 4.00 (3.00-4.03) | 14.4 (11.5-16.2) | 2.05 (74) |
| 30 mg (n = 6) | 33000 | (33.0) | 1800 | (23.7) | 4.00 (3.00-6.00) | 15.7 (11.3-23.1) | 1.78 (35) |
| 60 mg (n = 6) | 50000 | (31.4) | 3300 | (33.2) | 4.00 (3.00-4.00) | 13.8 (10.5-16.9) | 1.53 (53) |

Parameters are presented to 3 significant figures as mean (% CV), except Tₘₐₓ and t_{1/2} which are presented as median (min-max range), fₑ is fraction of dose excreted in urine as unmodified compound 2.

Pharmacodynamics

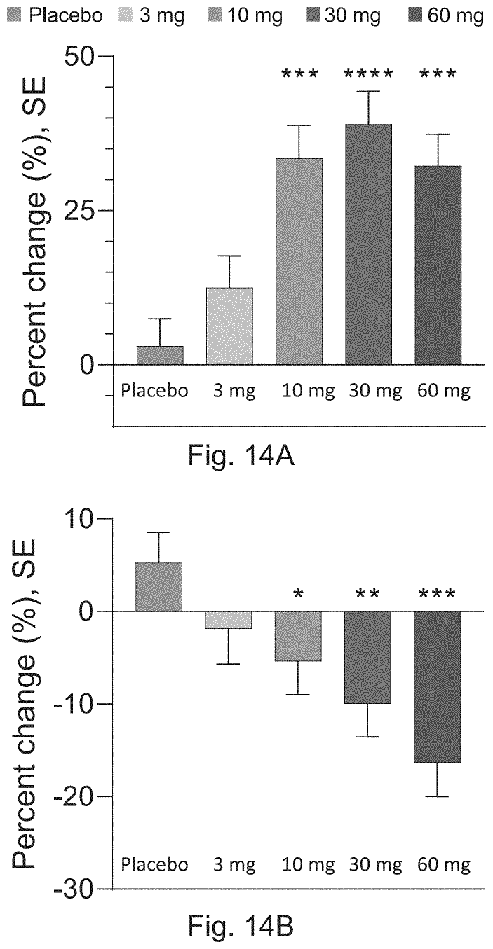

Mean percent change in sex hormone binding globulin (SHBG) and apolipoprotein B (Apo B) at Day 3 after a single dose of compound 2 on Day 1 of the study are shown in FIG. 14A and FIG. 14B, respectively. Mean percent change refer to least squares mean (LSM) from ANCOVA model and standard error (SE). P-value vs. placebo: *<0.05;**<0.01;***<0.001; ****<0.0001. Significant increases in SHBG were observed following single doses of ≥10 mg compound 2 relative to placebo. Dose-dependent decreases in LDL-c, total cholesterol, and Apo-B were observed on Day 3 following single dose administration of compound 2 with similar results on Day 4. No significant changes in triglyceride levels were observed after a single dose of compound 2 (see Table 14).

Table 14: PD marker changes, Day 3

| | | | | |
|---|---|---|---|---|
| Placebo (PBO) | 3 mg | 10 mg | 30 mg | 60 mg |
| (n = 8) | (n = 6) | (n = 6) | (n = 6) | (n = 6) |

SHBG

| | | | | | | | | | | |
|---|---|---|---|---|---|---|---|---|---|---|
| Baseline (nmol/L) | 35.0 | (17.97) | 46.7 | (26.30) | 55.0 | (20.63) | 26.0 | (11.19) | 39.3 | (31.97) |
| Change (nmol/L) | −0.77 | (2.17) | 0.20 | (2.51) | 15.8 | (2.61) | 6.0 | (2.6) | 5.05 | (2.49) |
| LSM % change | −3.8 | (3.91) | 6.3 | (4.52) | 31.3 | (4.69) | 16.5 | (4.67) | 13.8 | (4.48) |

| | | | | | |
|---|---|---|---|---|---|
| P-value vs. PBO | — | 0.11 | <0.0001 | 0.0023 | 0.0064 |

LDL-c

| | | | | | | | | | | |
|---|---|---|---|---|---|---|---|---|---|---|
| Baseline (nmol/L) | 106.1 | (36.06) | 137.5 | (17.4) | 133.7 | (18.2) | 123.3 | (23.55) | 125.2 | (45.96) |

| | | | | | | | | | |
|---|---|---|---|---|---|---|---|---|---|
| Change (nmol/L) | 5.8 | (3.44) | −3.98 | (3.39) | 0.68 | (3.34) | −15.7 | (3.32) | N/A |
| LSM % change | 5.6 | (2.85) | −3.1 | (2.80) | 0.70 | (2.76) | −12.5 | (2.75) | N/A |

| | | | | | |
|---|---|---|---|---|---|
| P-value vs. PBO | — | 0.048 | 0.24 | 0.0002 | N/A |

HDL-c

| | | | | | | | | | | |
|---|---|---|---|---|---|---|---|---|---|---|
| Baseline (nmol/L) | 45.4 | (11.40) | 51.0 | (22.53) | 63.7 | (23.55) | 49.5 | (7.82) | 48.2 | (13.93) |

| | | | | | | | | | |
|---|---|---|---|---|---|---|---|---|---|
| Change (nmol/L) | 0.29 | (0.93) | 0.87 | (0.91) | −1.1 | (0.96) | −6.1 | (0.91) | N/A |
| LSM % change | 1.7 | (1.77) | 2.4 | (1.73) | −1.0 | (1.83) | −12.1 | (1−74) | N/A |

| | | | | | |
|---|---|---|---|---|---|
| P-value vs. PBO | — | 0.78 | 0.3027 | <0.0001 | N/A |

TC

| | | | | | | | | | | |
|---|---|---|---|---|---|---|---|---|---|---|
| Baseline (nmol/L) | 171.7 | (45.05) | 216.5 | (29.87) | 217.7 | (38.10) | 196.2 | (31−2) | 191.2 | (51.68) |

| | | | | | | | | | |
|---|---|---|---|---|---|---|---|---|---|
| Change (nmol/L) | 2.3 | (4.73) | −1.85 | (4.59) | −1.93 | (4.61) | −24.14 | (4.52) | N/A |
| LSM % change | 1.6 | (2.15) | −0.88 | (2.09) | −0.84 | (2.09) | −11.84 | (2.06) | N/A |

| | | | | | |
|---|---|---|---|---|---|
| P-value vs. PBO | — | 0.44 | 0.45 | 0.0002 | N/A |

Apo B

| | | | | | | | | | | |
|---|---|---|---|---|---|---|---|---|---|---|
| Baseline (nmol/L) | 86.7 | (23.18) | 113.5 | (20.06) | 105.8 | (9.26) | 101.2 | (15.89) | 92.3 | (24.9) |
| Change (nmol/L) | 3.8 | (2.72) | −1.5 | (3.14) | −5.6 | (3.0) | −11.5 | (2.97) | −13.9 | (3.0) |
| LSM % change | 5.5 | (2.63) | −2.7 | (3.04) | −6.0 | (2.91) | −11.0 | (2.9) | −15.75 | (2.91) |

| | | | | | |
|---|---|---|---|---|---|
| P-value vs. PBO | — | 0.064 | 0.0081 | 0.0003 | <0.0001 |

### Conclusions

Single ascending doses of compound 2 up to 60 mg were overall safe and well-tolerated and exhibited linear and dose-proportional plasma exposures with low variability. The half-life was >13 hours at all single dose levels, supportive of once daily oral dosing. Renal excretion of unchanged compound 2 was minimal, indicating renal elimination is a minor pathway.

Significant dose-dependent effects on SHBG, Apo B, and LDL-c were observed following a single dose of compound 2, indicating the potential for efficacy. Relative to the efficacious dose of compound 2 in preclinical models (threshold AUC of 3,320 ng*h/mL achieved significant histological improvement in a mouse model of NASH), sufficient exposure levels were achieved in human subjects across all compound 2 doses.

The safety, PK, and PD results support continued development of compound 2 and indicate that it is well-suited for co-formulation with other oral small molecule NASH agents as an oral, once-daily fixed dose combination.

### Example 9: Multiple Ascending Dose Trial of Compound 2 in Healthy Human Subjects

A multiple-ascending dose clinical trial of Compound 2 was performed. Four groups of 8 healthy participants were randomized to receive Compound 2 (1 mg, 3 mg, 6 mg, or 10 mg capsule) or matching placebo in a 3:1 ratio (n=6 active and n=2 placebo) and were administered once daily during the fasted state for 14 days. Plasma levels of Compound 2 and PD biomarkers were determined at pre-dose and various time points post-dose.

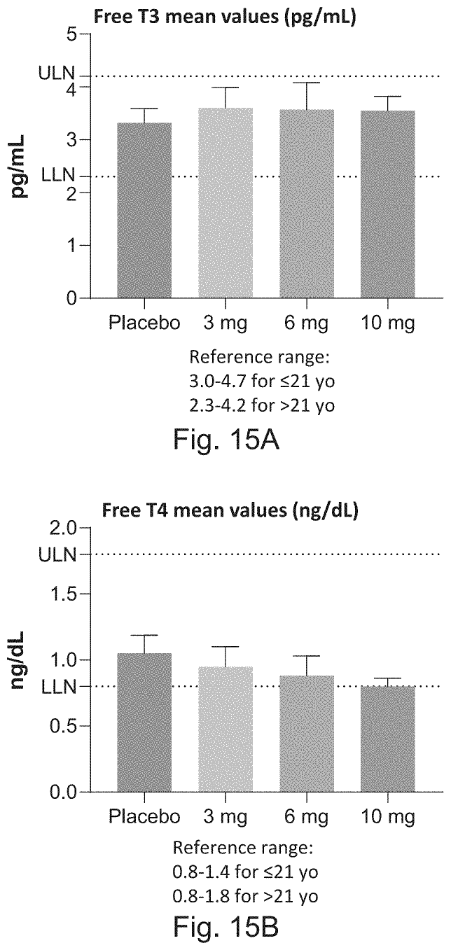

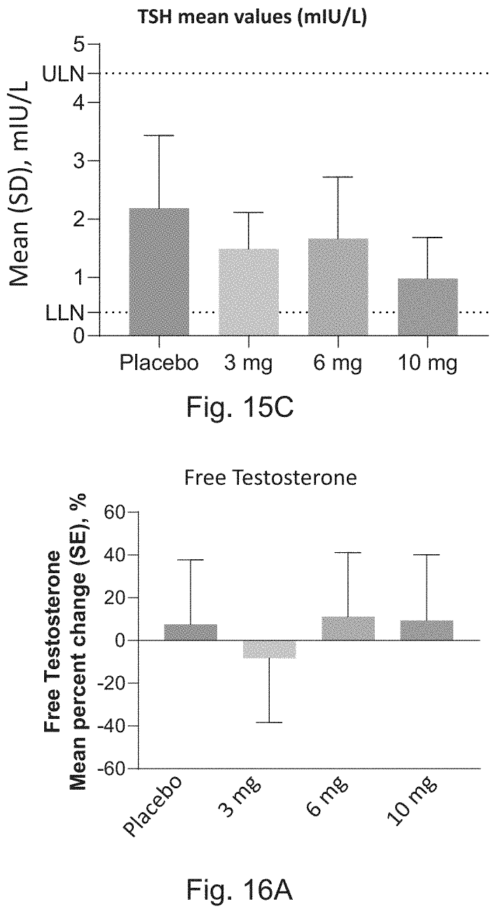

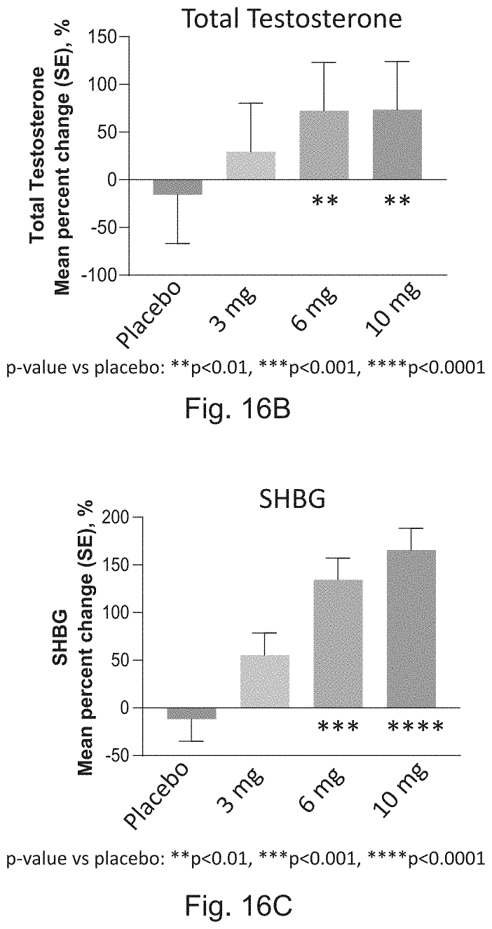

Adverse event (AE) monitoring (Table 15), routine clinical laboratory testing (including thyroid axis testing [free and total thyroid hormone triiodothyronine (T3), free and total thyroid hormone thyroxine (T4), thyroid stimulating hormone (TSH)] cardiac biomarkers [CK-MB, troponin I], and liver biochemistry) (FIG. 15A-C, FIG. 16A-C), intensive vital signs, cardiac telemetry, and electrocardiograms were assessed throughout the study. Compound 2 plasma and urine concentrations were determined using validated liquid chromatography-tandem mass spectrometry assay.

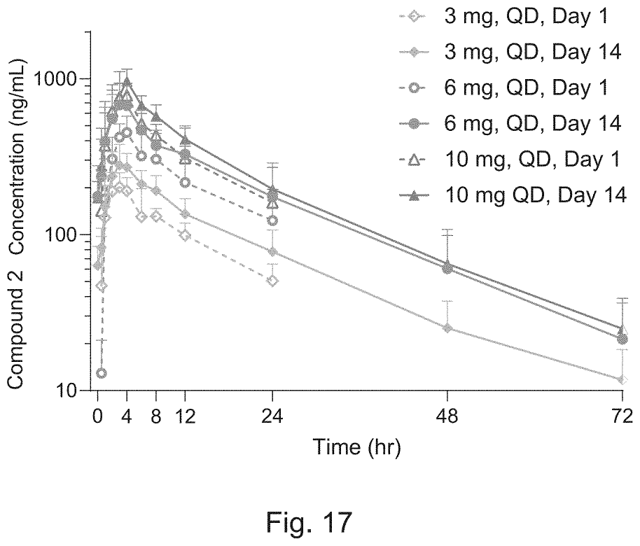

Plasma samples for Compound 2 concentration and PK sampling were collected pre-dose and at 0.5, 1, 2, 3, 4, 6, 8, 12, and 24 hours after the first dose, at pre-dose on Days 3, 4, 5, 8, 11, and 13 of dosing, and at pre-dose and at 0.5, 1, 2, 3, 4, 6, 8, 12, 24, 48, and 72 hours after 14 days of administration of study medication (placebo or compound). (See Table 16, FIG. 17) PK parameters were estimated via noncompartmental methods using Phoenix® WinNonlin® (Certara, LP, Princeton, N.J.). Concentrations of serum pharmacodynamic (PD) biomarkers apolipoprotein B (Apo B) and sex hormone binding globulin (SHBG) were measured using an immunoassay and serum lipids were determined using spectrophotometry.

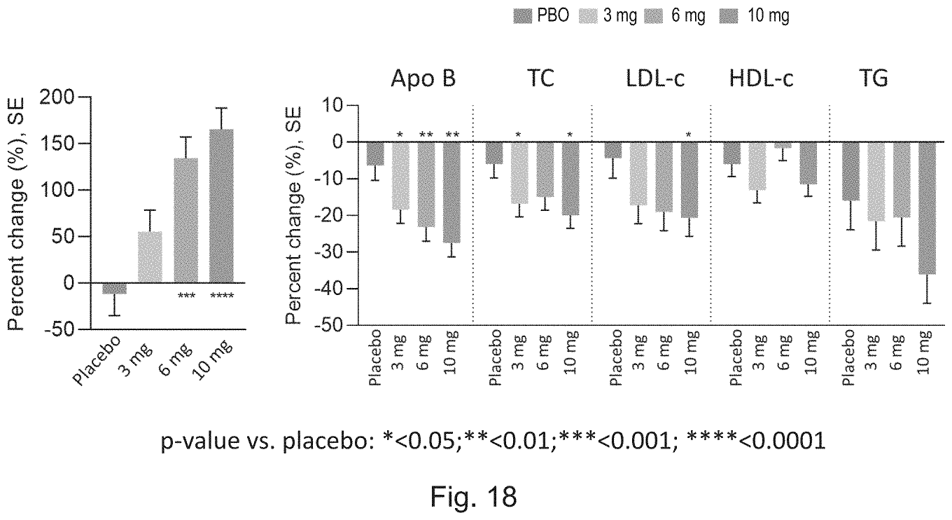

PD sampling was also performed. Percent change from baseline for PD markers were calculated using an ANCOVA model with percent change from baseline as dependent variable, treatment group as fixed effect, and baseline as covariate. Analyses used observed data only without imputation for missing data. PD data on day 15 of the study are shown in FIG. 18.

### RESULTS AND CONCLUSIONS

### Safety

TABLE 15
Safety and adverse effects

| | Placebo | 3 mg | 6 mg | 10 mg |
|---|---|---|---|---|
| Subject Incidence | (N = 6) | (N = 6) | (N = 6) | (N = 6) |
| AEs by category | n (%) | n (%) | n (%) | n (%) |
| Any AE, all CTCAE grades | 0 | 1 (16.7%) | 1 (16.7%) | 2 (33.3%) |
| Grade 1 | 0 | 1 (16.7%) | 1 (16.7%) | 2 (33.3%) |
| Grade 2 or higher | 0 | 0 | 0 | 0 |
| Serious AEs | 0 | 0 | 0 | 0 |

AEs by relationship to drug

| | | | | |
|---|---|---|---|---|
| Not related | 0 | 1 (16.7%) | 0 | 2 (33.3%) |
| Unlikely related | 0 | 0 | 0 | 0 |
| Possibly related | 0 | 0 | 1 (16.7%) | 0 |
| Related | 0 | 0 | 0 | 0 |

*Dizziness was reported in one subject;
CTCAE = common terminology criteria for adverse events, AE = adverse event; Treatment-emergent adverse events (TEAE) are those that occur on or after the time of first dose of study drug; Severity of adverse events was graded according to CTCAE version 5.

14-day once-daily dosing of placebo or Compound 2 up to 10 mg was overall well-tolerated. No study stopping criteria or dose escalation stopping criteria were met. All AEs were Grade 1 and most considered not related to the study drug. All subjects randomized to Compound 2 completed the study with no study drug discontinuation. Heart rate and blood pressure remained stable throughout the study. Free T4 declined in dose-dependent fashion without clear change in TSH or free T3; T4 changes were asymptomatic and not considered clinically significant, suggesting peripheral thyroid hormone modulation leading to lower free T4 without changes in T3 or TSH.

Mean ALT values were similar across the groups, and not significantly different from placebo; no treated subject had ALT increase to ≥2×ULN. Dose-dependent GGT increases were noted, with values remaining below ULN for treated patients. Dose-dependent total testosterone increases were observed, but no significant change in free levels was identified.

No remarkable changes in ECGs, cardiac biomarkers, or other clinical lab tests were identified.

### Pharmacokinetics and Pharmacodynamics

TABLE 16
Pharmacokinetics

| | | | 3 mg, QD | | 6 mg, QD | | 10 mg, QD | |
|---|---|---|---|---|---|---|---|---|
| Day 14 PK Parameter | 1 mg, QD | | N = 6 | | N = 6 | | N = 6 | |
| AUCₜₐᵤ (hr · ng/mL) | 1090 | (15.7) | 3600 | (26.9) | 8310 | (44.4) | 10600 | (21.1) |
| (mean, % CV) | | | | | | | | |
| Cₘₐₓ (ng/mL) | 80.3 | (20.0) | 291 | (30.9) | 699 | (35.1) | 996 | (18.0) |
| (mean, % CV) | | | | | | | | |
| Cₜₐᵤ (ng/mL) | 25.7 | (28.6) | 77.9 | (37.6) | 175 | (64.7) | 196 | (38.0) |
| (mean, % CV) | | | | | | | | |
| t_{1/2} (hr) | 19.5 | (17.0-23.8) | 16.0 | (14.1-19.6) | 15.8 | (12.7-17.6) | 15.4 | (13.1-18.5) |
| (median, min-max) | | | | | | | | |

Pharmacokinetic data indicated good oral bioavailability with low PK variability. Multiple doses of Compound 2 led to significant and dose-dependent increases in SHBG, even at a low dose of 3 mg QD. Reductions in total cholesterol, LDL-c, Apo B, and triglycerides were observed in all dose levels of Compound 2, with significant reductions observed at day 15 in the 10 mg dose cohort. HDL-c did not significantly change in by day 15. These results support a low once daily dose of Compound 2 is efficacious.

### Example 10: Drug-Drug Interaction Studies with Compound 2 in Healthy Humans

In vitro studies indicate compound 2 has potential for first pass inhibition of organic anion transporting polypeptides (OATP) 1B1/1B3 and intestinal inhibition of breast cancer resistance protein (BCRP) transporters (OATP1B1 IC50=2.01 micromolar; OATP1B3 IC50=0.71 micromolar; BCRP IC50=9.37 micromolar). The effect of compound 2 on the metabolism of coadministered rosuvastatin (ROS), an antihyperlipidemia drug and a substrate of OATP and BCRP, in healthy participants, is determined by administering Compound 2 and ROS as described in Table 17, in combination with PK sampling.

TABLE 17

| Study Day | 1 | 2 | 3-8 | 9 | 10-11 | 12 |
|---|---|---|---|---|---|---|
| Cohort 1 | Single- | No | Up to | Single- | No | Clinic |
| (n = 10) | dose of | Treatment | 90 mg | dose of | Treatment | Discharge |
| | 10 mg | | Compound | up to 90 mg | | |
| | ROS PO | | 2, QD PO | Compound 2 + | | |
| | | | | Single-dose | | |
| | | | | of 10 mg | | |
| | | | | ROS PO | | |

PO = by mouth; QD = once daily

In addition, Compound 3 is an inhibitor of intestinally expressed transporters P-glycoprotein (P-gp) and BCRP (P-gp IC50=3.92 micromolar; BCRP IC50=4.39 micromolar). Based on in vitro studies, inhibition of these transporters by Compound 3 has the potential to increase the absorption of coadministered Compound 2. Thus, a drug-drug interaction (DDI) study to assess the potential for Compound 3 to enhance the absorption of Compound 2 via inhibition of intestinal P-gp and BCRP is conducted, as described in Table 18.

TABLE 18

| Study Day | 1 | 2-6 | 7-12 | 13 | 14-16 | 17 |
|---|---|---|---|---|---|---|
| Cohort 2 | Single- | No | 15 mg | Single- | No | Clinic |
| | dose of | Treatment | Compound | dose of | Treatment | Discharge |
| | up to | | 3 QD PO | up to 90 mg | | |
| | 90 mg | | | Compound 2 + | | |
| | Compound | | | Single dose | | |
| | 2 PO | | | of 15 mg | | |
| | | | | Compound 3 | | |

### Example 11: Food Effect on Compound 2 in Healthy Humans

The food effect on uptake and pharmacokinetics of compound 2 in healthy participants is determined by administering compound 2 during the fed and fasted state, as described in Table 19, in combination with PK sampling.

TABLE 19

| | Study Period/Study Day | | | | |
|---|---|---|---|---|---|
| | Period 1/Day 1 | Days 2-8 | Period 2/Day 9 | Days 10-11 | Day 12 |
| Cohort 3 | Up to 90 mg | Washout | Up to 90 mg | No | Clinic |
| Sequence A | Compound 2 PO | | Compound 2 | Treatment | Discharge |
| (n = 4) | (Fasted) | | (Fed) | | |
| Cohort 3 | Up to 90 mg | Washout | Up to 90 mg | No | Clinic |
| Sequence B | Compound 2 PO | | Compound 2 PO | Treatment | Discharge |
| (n = 4) | (Fed) | | (Fasted) | | |

Participants undergo an overnight fast (no food or liquids, except water, for at least 10 hours prior to dosing). On Day 9 in Sequence A, and Day 1 in Sequence B, a high-fat/high-calorie breakfast containing 1000 kcal and 45% to 55% fat is provided 30 minutes prior to study drug administration. Study drug is administered at or within 5 minutes after participants complete (100% consumption) the breakfast.

Thyroid axis safety monitoring and cardiovascular safety monitoring are conducted as described in the preceding Examples.

### Example 12 Combination Therapy of Compound 1 and Compound 3 in Rat Model of NASH

Animal handling: After arrival, the rats were left for a 2-week acclimation period, during which they were accustomed to the animal facility staff and trained on the procedure of oral gavage. After 2 weeks the animals were put on CHDFD (choline deficient high fat diet) and pre-fed for 4 weeks. Then the rats were started on treatment with test compounds, and 3× per week i.p. NaNO₂ injections, while they remained on CDHFD, for an additional 8 weeks. NaNO₂ was administered at 25 mg/kg i.p. dissolved in PBS 3 times a week (on Mondays, Wednesdays, and Fridays) for 8 weeks while on CDHFD.

Final Sacrifice: Half of the animals of each treatment group were terminated on day 84. The other half of the animals in each group were terminated on the following day, day 85. On the day of sacrifice the animals were fasted for 2 h and received a final treatment with the respective test substance. After the final compound treatment, the animals had no more access to food until sacrifice. At 4 h after the last administration all animals were sacrificed and livers were sampled for further analysis.

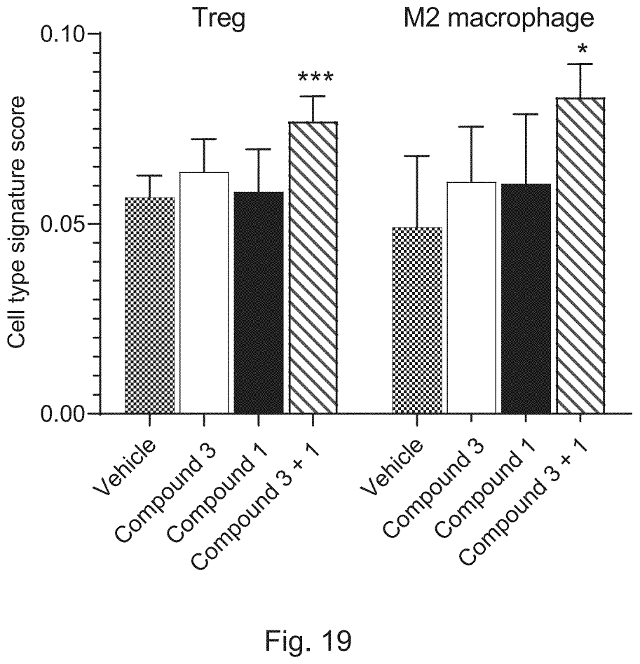

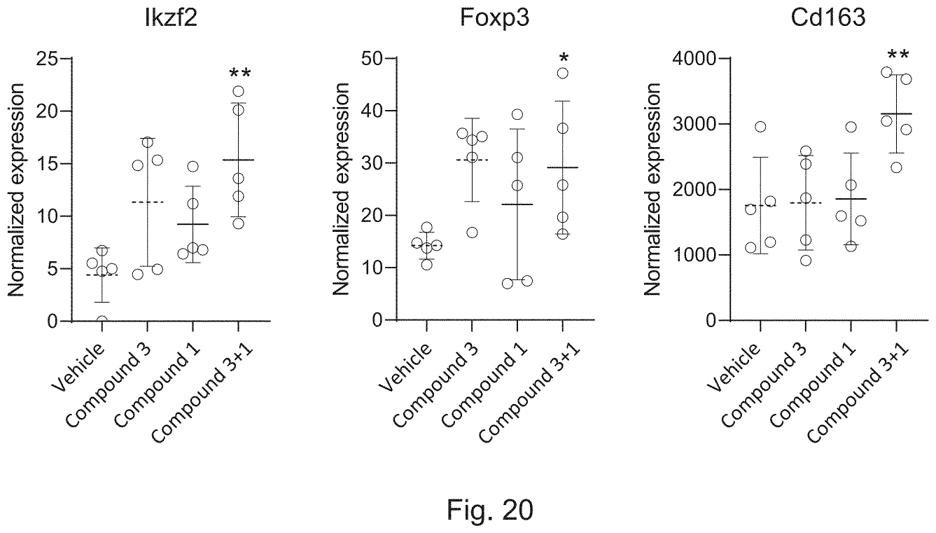

RESULTS: The choline-deficient, high-fat diet (CDHFD) is commonly used to induce a NASH-like phenotype in rodent species. In addition, induction of liver fibrosis by intraperitoneal (IP) injections of sodium nitrite (NaNO₂) in CDHFD rats can be used to model advanced NASH disease. Therefore, the rat CDHFD+NaNO₂ NASH model was used to test the efficacy of Compound 3 alone and in combination with Compound 1. In this model male Wistar rats were fed a CDHFD for 4 weeks to induce disease prior to daily oral drug and triweekly IP NaNO₂ treatment. Following 8 weeks of Compound 3 (3 mg/kg) and Compound 1 (25 mg/kg) dosing as single agents or in combination, liver tissue was processed for whole transcriptome analysis by RNAseq to look for changes in gene expression associated with disease resolution. In NASH, resolution is a complex process that involves liver infiltration of specialized cells of the immune system including regulatory T cells (Treg) and M2 macrophages. Treg and M2 macrophages are involved in immune suppression and reducing inflammation and appear to have a beneficial role in animal models of liver injury including NASH. To look for the presence of these cells, we utilized RNAseq expression data to perform single-sample gene set enrichment analysis (ssGSEA) using cell type specific gene expression signatures to quantitate relative levels of Treg and M2 macrophage infiltration into the liver (FIG. 19). The combination of Compound 3 (3 mg/kg) and Compound 1 (25 mg/kg) showed significantly higher scores for both Treg and M2 macrophages relative to vehicle-treated NASH control animals. In contrast, single agent treated animals were not significantly different from control. These results were verified (FIG. 20) by analysis of individual markers of Treg and M2 macrophages including Foxp3 (Treg), Ikzf2 (Treg), and Cd163 (M2 macrophage). Only the combination of Compound 3 (3 mg/kg) and Compound 1 (25 mg/kg) showed significantly higher expression of markers associated with Treg and M2 macrophage cells. Taken together these data suggest that the combination of Compound 3, an FXR agonist, and Compound 1, an inhibitor of SSAO, resulted in increased expression of immune cell markers in the liver that are associated with NASH resolution. Given their distinct mechanisms of action, Compound 3 and Compound 1 could provide complementary benefits when used in combination to accelerate NASH resolution processes.

These results demonstrate that the combination of a FXR agonist and an SSAO inhibitor combine to have an effect that is greater than either of the two drugs administered singly.

### Example 13: Differential Gene Expression in a Combination Therapy of Compound 1 and Compound 3 in Rat Model of NASH

A study was performed to show the beneficial effects of combining a FXR agonist and an SSAO inhibitor in a rat model of NASH.

Animal handling: After arrival, the rats were left for a 2-week acclimation period, during which they were accustomed to the animal facility staff and trained on the procedure of oral gavage. After 2 weeks the animals were placed on a choline deficient high fat diet (CDHFD) and pre-fed for 4 weeks to induce steatosis and a NASH-like disease phenotype. Rats were then treated with test compounds for an additional 8 weeks while on CDHFD. Concomitant with compound treatment, rats were administered sodium nitrite (NaNO₂, 25 mg/kg dissolved in PBS) by triweekly intraperitoneal (IP) injection to induce liver fibrosis.

Final Sacrifice: Half of the animals of each treatment group were terminated on day 84. The other half of the animals in each group were terminated on the following day, day 85. On the day of sacrifice the animals were fasted for 2 hours and received a final treatment with the respective test substance. After the final compound treatment, the animals had no more access to food until sacrifice. At 4 hours after the last administration all animals were sacrificed and livers were sampled for further analysis.

Sampling and analysis: Small liver pieces were harvested into RNAlater (Thermo Fisher Scientific Dreieich Germany) and stored at −20° C. prior to RNA sequencing (RNAseq) at MedGenome Inc. RNAseq analysis was performed on liver tissue by Illumina sequencing using standard methodologies. Briefly, RNAseq libraries (n=5 per group) were generated using Illumina Truseq stranded mRNA kits and sequencing was performed on a NovaSeq 6000 sequencer. Alignment was performed using STAR (v2.7.3a) aligner and reads mapping to ribosomal and mitochondrial genome were removed prior to alignment. Raw read counts were estimated using HTSeq (v0.11.1) and normalized using DESeq2 (v2.22.2). Differentially expressed genes (DEGs) were determined using DESeq2 (R Bioconductor package).

### Results

The choline-deficient, high-fat diet (CDHFD) is commonly used to induce a NASH-like phenotype in rodent species. In addition, induction of liver fibrosis by intraperitoneal (IP) injections of sodium nitrite (NaNO₂) in CDHFD rats can be used to model advanced NASH disease. Therefore, the rat CDHFD+NaNO₂ NASH model was used to test the efficacy of Compound 3 alone and in combination with Compound 1. In this model, male Wistar rats were fed a CDHFD for 4 weeks to induce disease prior to daily oral drug and triweekly IP NaNO₂ treatment. Following 8 weeks of Compound 3 (3 mg/kg) and Compound 1 (25 mg/kg) dosing as single agents or in combination, liver tissue was processed for whole transcriptome analysis by RNAseq. Table 20 shows the total number and change direction (i.e., up or down relative to vehicle control) of differentially expressed genes (DEGs) identified in CDHFD+NaNO₂ rats treated with Compound 3 (3 mg/mg), Compound 1 (25 mg/kg), or the combination of Compound 3 (3 mg/kg) and Compound 1 (25 mg/kg). Using an absolute fold-change cutoff of ≥1.5-fold and adjusted p-value of <0.01, 309 DEGs were identified in Compound 3 treatment group, 847 DEGs were identified in Compound 1 treated animals, and 1351 DEGs were identified in the combination treatment group. These results suggest that the combination treatment resulted in at least additive effects on the total number of DEGs relative to single agent treatment groups.

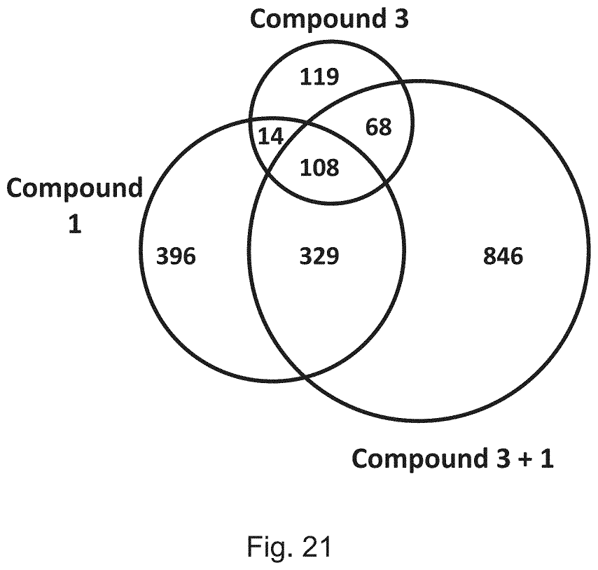

Surprisingly, a larger number of upregulated DEGs were observed in the combination treatment group relative to individual treatment arms. FIG. 21 shows the number and overlap of DEGs (vs. vehicle NASH control) identified in each treatment group using absolute fold-change and adjusted p-value cutoffs of ≥1.5 and <0.01, respectively.

TABLE 20
Differentially expressed genes (DEGs)

| Treatment group | Down DEGs | Up DEGs | Total DEGs |
|---|---|---|---|
| Compound 3 (3 mg/kg) | 118 | 191 | 309 |
| Compound 1 (25 mg/kg) | 641 | 206 | 847 |
| Compound 3 (3 mg/kg) + | 724 | 627 | 1351 |
| Compound 1 (25 mg/kg) | | | |

Number of DEGs identified (vehicle NASH control vs. treatment) identified for each treatment group. Adjusted p-value<0.01 and fold-change ≥1.5-fold.

We next examined the differential expression of genes associated with lipid metabolism and triglyceride accumulation that were previously described (Shepherd E, Karim S, Newsome P, and Lalor P., Inhibition of vascular adhesion protein-1 modifies hepatic steatosis in vitro and in vivo. World J Hepatol. 2020 12(11): 931-948). Compound 1 treatment resulted in statistically significant changes in the expression of genes related to lipid metabolism and fatty-acid transportation including Vldlr, Fabp2, Vegfc, Ldlrap1, Ldlr, Ppargc1a, and Slc27a5 (Table 21, denoted by asterisk). Of these, Vldlr, Fabp2, and Slc27a5 were changed by ≥1.5-fold (shown in bold). Only Fabp2 was significantly differentially expressed upon treatment with Compound 3. Interestingly, the combination of Compound 3 and Compound 1 resulted in substantially more DEGs related to lipid metabolism and fatty-acid transportation than either single agent treatment group. Moreover, several genes were differentially expressed by ≥1.5-fold relative to vehicle control, including Vldlr, Fabp2, Il1r2, Ppara, Ldlr, Ppargc1a, Rxra, and Slc27a5.

Taken together these data suggest that the combination of Compound 3, an FXR agonist, and Compound 1, an inhibitor of SSAO, resulted in significant changes in the expression of genes involved in lipid metabolism and fatty-acid transport. Moreover, the pattern of gene expression changes is largely consistent with an enhanced anti-steatotic effect relative to treatment with Compound 3 alone. Given their distinct mechanisms of action, Compound 3 and Compound 1 could provide complementary benefits when used in combination to accelerate NASH resolution processes.

TABLE 21
Differentially expressed genes associated with
lipid metabolism and fatty acid transport

| | Differential gene expression analysis | | |
|---|---|---|---|
| | (log₂-fold change) relative to vehicle control | | |
| | Compound 1 | Compound 3 | Compound 1 + Compound 3 |
| Gene | (25 mg/kg) | (3 mg/kg) | (25 mg/kg + 3 mg/kg) |
| Vldlr | −1.6* | −0.58 | −1.17* |
| Fabp2 | −1.02* | −1.02* | −1.2* |
| Il1r2 | −0.45 | −0.05 | −0.95* |
| Vegfc | −0.45* | −0.28 | −0.54* |
| Lrp2 | 0.08 | 0.33 | 0.32* |
| Irs2 | 0.13 | 0.27 | 0.41* |
| Vegfa | 0.23 | 0.48 | 0.41* |
| Lrp1 | 0.25 | 0.51 | 0.48* |
| Irs1 | 0.26 | −0.03 | 0.45* |
| Ppara | 0.32 | 0.36 | 0.68* |
| Slc27a1 | 0.33 | 0.16 | 0.51* |
| Ldlrap1 | 0.38* | −0.07 | 0.32* |
| Ldlr | 0.41* | 0.4 | 0.67* |
| Ppargcla | 0.51* | 0.25 | 0.85* |
| Rxra | 0.51 | 0.03 | 0.62* |
| Slc27a5 | 0.68* | 0.5 | 0.81* |

Gene expression analysis (RNAseq) in the liver of CDHFD+NaN0₂ rats. Log₂-fold-change relative to vehicle control for genes involved in lipid metabolism and fatty-acid transportation. Negative change direction (−) indicates decreased expression by treatment relative to vehicle; positive change direction indicates increased gene expression relative to vehicle control. Absolute fold-change values ≥1.5-fold (i.e., log 2-fold change ≥0.6 or ≤−0.6) indicated in bold. *p-value<0.05.

### Example 14: Differentially Expressed Genes (DEGs) in a Combination Therapy of Compound 2 and Compound 3 in Rat Model of NASH

C57BL/6J mice were fed a high fat diet for 10 weeks to induce obesity (>38 g BW). Obese mice were injected intraperitoneally (i.p.) twice a week for four weeks with 0.5 nl/g 25% CCl₄ (formulated in olive oil) to induce fibrosis, and one group of normal BW mice were injected i.p. twice a week for four weeks with olive oil to serve as a healthy control. During the same dosing period, obese mice were fed orally once a day for 28 days with vehicle, Compound 3 or Compound 2 as single agents or in combination. On CCl₄ dosing days, CCl₄ was administered at 4 hours post compound or vehicle dosing. On day 27, all animals were fasted for about 16 hours before terminal euthanasia. On day 28, all animals were sacrificed and liver samples were collected for whole transcriptome analysis by RNA sequencing (RNAseq). RNAseq library (n=5 per group) preparation and sequencing was performed using Illumina standard protocols. Alignment of sequencing reads was performed using STAR aligner software and read counts were estimated using RSEM. Differentially expressed genes (compared to vehicle-treated NASH control mice) were determined using EdgeR software. Gene ontology analysis was performed using Advaita software with fold-change and adjusted p-value cutoffs of >1.5 and <0.05, respectively. Gene ontologies were derived from the Gene Ontology Consortium database (2019 Apr. 26) (Ashburner et al., Gene ontology: Tool for the unification of biology. Nature Genetics 25(1): 25-9 (2000); Gene Ontology Consortium, Creating the Gene Ontology Resource: Design and Implementation. Genome Research 11: 1425-1433 (2001)).

The change direction (i.e., up or down) and total number of differentially expressed genes (DEGs) identified between vehicle-treated NASH controls and mice treated with Compound 3 (3 mg/mg), Compound 2 (1 mg/kg), or the combination of Compound 3 (3 mg/kg) and Compound 2 (1 mg/kg) are shown in Table 22. Using an absolute fold-change cutoff of >1.5-fold and adjusted p-value of <0.05, 617 DEGs were identified in Compound 3 treated mice, 1113 DEGs were identified in Compound 2 treated mice, and 1871 DEGs were identified in mice treated with the combination of Compound 3 and Compound 2. These results suggest that the combination treatment resulted in at least additive effects on the total number of DEGs relative to the arithmetic sum of DEGs identified from each single treatment group. The number of down regulated DEGs (Down DEGs) was higher in the combination treatment group compared to the arithmetic sum of Down DEGs from each single agent treatment group. These results indicated that the combination of Compound 3 and Compound 2 resulted in a larger than expected number of DEGs relative to single agent treatments and this effect was the result of a larger than expected number of down regulated DEGs.

TABLE 22
Differentially expressed genes (DEGs)

| Treatment group | Down DEGs | Up DEGs | Total DEGs |
|---|---|---|---|
| Compound 3 (3 mg/kg) | 271 | 346 | 617 |
| Compound 2 (1 mg/kg) | 635 | 478 | 1113 |
| Compound 3 (3 mg/kg) + | 1182 | 689 | 1871 |
| Compound 2 (1 mg/kg) | | | |

Number of DEGs identified (vehicle NASH control vs. treatment) identified for each treatment group. Adjusted p value<0.05 and fold-change >1.5-fold

### Example 14.1: Gene Ontology (GO) Enrichment Analysis

Gene ontology (GO) enrichment analysis was used to understand the potential biological consequences of the results in Table 22. To perform GO term enrichment analysis, the number (i.e., enrichment) of DEGs annotated for a particular term (i.e. biology process) was compared to the number of DEGs expected solely by chance. An over-representation approach was used to compute statistical significance (p-value) of observing at least the given number of DEGs; p-values were corrected for multiple comparisons.

Liver inflammation is a defining characteristic and key driver of NASH disease and is mediated in large part by overactivation and infiltration of leukocytes into the liver. Therapies that target inflammatory processes directly via anti-inflammatory mechanisms or indirectly by, for example, decreasing oxidative stress by normalizing metabolic function and reducing liver steatosis, have the potential to impact NASH disease. Table 23 shows GO term enrichment analysis for DEGs associated with leukocyte-related biological processes. As shown in Table 23, only the combination of Compound 3 and Compound 2 showed a statistically significant enrichment of DEGs associated with leukocyte-related biological processes. These results suggested that the combination of Compound 3 with Compound 2 had a much more profound effect on leukocyte-related biological processes than either single treatment alone.

TABLE 23
GO term enrichment analysis for leukocyte-related biological processes

| | | Compound 3 | Compound 2 | Compound 3 + |
|---|---|---|---|---|
| Biological process | GO ID | (3 mg/kg) | (1 mg/kg) | Compound 2 |
| myeloid leukocyte activation | GO:0002274 | 0.52 | 0.36 | 1.6E−08 |
| leukocyte activation | GO:0045321 | 0.73 | 0.45 | 5.8E−08 |
| leukocyte migration | GO:0050900 | 0.47 | 0.36 | 2.3E−07 |
| leukocyte activation involved | GO:0002269 | 0.38 | 0.1 | 5.1E−06 |
| in inflammatory response | | | | |
| myeloid leukocyte migration | GO:0097529 | 0.74 | 0.52 | 1.1E−05 |
| leukocyte chemotaxis | GO:0030595 | 0.65 | 0.45 | 2.6E−05 |
| leukocyte cell-cell adhesion | GO:0007159 | 0.58 | 0.36 | 6.9E−05 |
| leukocyte proliferation | GO:0070661 | 0.79 | 0.62 | 9.4E−05 |
| regulation of leukocyte migration | GO:0002685 | 0.49 | 0.25 | 0.00017 |
| leukocyte mediated immunity | GO:0002443 | 0.71 | 0.84 | 0.00018 |

Adjusted p-values shown for each treatment group. Top ten leukocyte-associated biological processes enriched in the Compound 3 and Compound 2 combination treatment group shown. Table 24 shows GO term enrichment analysis for DEGs associated with immune and leukocyte-related biological processes that were uniquely enriched by combination treatment as described in Example 14.

TABLE 24
GO term enrichment analysis of immune-related biological
pathways uniquely enriched by combination treatment

| | GO term | DEG count | Total Genes | Corrected |
|---|---|---|---|---|
| Biological process | ID | (n) | (n) | p-value |
| immune response | GO:0006955 | 216 | 941 | 1.21E−10 |
| inflammatory response | GO:0006954 | 124 | 467 | 1.12E−09 |
| myeloid leukocyte activation | GO:0002274 | 55 | 156 | 1.59E−08 |
| immune system process | GO:0002376 | 327 | 1674 | 3.94E−08 |
| leukocyte activation | GO:0045321 | 145 | 615 | 5.79E−08 |
| positive regulation of immune | GO:0002684 | 156 | 687 | 1.86E−07 |
| system process | | | | |
| leukocyte migration | GO:0050900 | 69 | 233 | 2.33E−07 |
| regulation of immune response | GO:0050776 | 132 | 567 | 5.75E−07 |
| regulation of immune system | GO:0002682 | 202 | 972 | 9.68E−07 |
| process | | | | |
| leukocyte activation involved | GO:0002269 | 18 | 32 | 5.1E−06 |
| in inflammatory response | | | | |
| myeloid leukocyte migration | GO:0097529 | 45 | 142 | 1.09E−05 |
| leukocyte chemotaxis | GO:0030595 | 44 | 142 | 2.6E−05 |
| positive regulation of immune | GO:0050778 | 104 | 455 | 3.94E−05 |
| response | | | | |
| innate immune response | GO:0045087 | 113 | 508 | 5.06E−05 |
| leukocyte cell-cell adhesion | GO:0007159 | 61 | 231 | 6.9E−05 |
| leukocyte proliferation | GO:0070661 | 59 | 223 | 9.41E−05 |
| neuroinflammatory response | GO:0150076 | 20 | 47 | 0.000173 |
| regulation of leukocyte migration | GO:0002685 | 43 | 148 | 0.000173 |
| leukocyte mediated immunity | GO:0002443 | 66 | 265 | 0.000185 |
| cell activation involved | GO:0002263 | 51 | 192 | 0.000323 |
| in immune response | | | | |
| leukocyte activation involved | GO:0002366 | 50 | 188 | 0.000373 |
| in immune response | | | | |
| regulation of leukocyte activation | GO:0002694 | 87 | 386 | 0.000383 |
| regulation of inflammatory | GO:0050727 | 63 | 256 | 0.000397 |
| response | | | | |
| positive regulation of leukocyte | GO:0002696 | 58 | 230 | 0.000406 |
| activation | | | | |
| adaptive immune response | GO:0002250 | 69 | 293 | 0.000639 |
| positive regulation of leukocyte | GO:0002687 | 32 | 106 | 0.000913 |
| migration | | | | |
| immune effector process | GO:0002252 | 114 | 554 | 0.001036 |
| positive regulation of | GO:0050729 | 29 | 93 | 0.001062 |
| inflammatory response | | | | |
| neutrophil activation involved | | 9 | 14 | 0.001102 |
| in immune response | | | | |
| immune response-activating signal | GO:0002757 | 59 | 246 | 0.001269 |
| transduction | | | | |
| regulation of leukocyte | GO:0070663 | 44 | 168 | 0.001381 |
| proliferation | | | | |
| immune response-regulating | GO:0002764 | 60 | 255 | 0.001816 |
| signaling pathway | | | | |
| leukocyte aggregation | GO:0070486 | 8 | 12 | 0.001944 |
| regulation of leukocyte mediated | GO:0002703 | 42 | 164 | 0.003108 |
| immunity | | | | |
| positive regulation of immune | GO:0002699 | 43 | 170 | 0.003403 |
| effector process | | | | |
| positive regulation of leukocyte | GO:1903039 | 38 | 145 | 0.003827 |
| cell-cell adhesion | | | | |
| regulation of leukocyte cell-cell | GO:1903037 | 50 | 208 | 0.003827 |
| adhesion | | | | |
| myeloid cell activation involved | GO:0002275 | 21 | 64 | 0.004329 |
| in immune response | | | | |
| positive regulation of leukocyte | GO:0002690 | 21 | 64 | 0.004329 |
| chemotaxis | | | | |
| leukocyte differentiation | GO:0002521 | 86 | 413 | 0.004907 |
| activation of immune response | GO:0002253 | 68 | 312 | 0.005594 |
| myeloid leukocyte mediated | GO:0002444 | 22 | 70 | 0.005847 |
| immunity | | | | |
| positive regulation of leukocyte | GO:0002705 | 29 | 104 | 0.006575 |
| mediated immunity | | | | |
| acute inflammatory response | GO:0002526 | 24 | 81 | 0.007746 |
| leukocyte degranulation | GO:0043299 | 17 | 50 | 0.00959 |
| regulation of leukocyte | GO:0002688 | 23 | 78 | 0.010243 |
| chemotaxis | | | | |
| immune response-activating cell | GO:0002429 | 35 | 140 | 0.012131 |
| surface receptor signaling pathway | | | | |
| regulation of myeloid leukocyte | GO:0002886 | 16 | 47 | 0.012275 |
| mediated immunity | | | | |
| immune response-regulating cell | GO:0002768 | 36 | 147 | 0.014639 |
| surface receptor signaling pathway | | | | |
| positive regulation of leukocyte | GO:0070665 | 26 | 99 | 0.023913 |
| proliferation | | | | |

Top 50 immune-related biological processes that were uniquely enriched by Compound 1 (3 mg/kg) and Compound 2 (1 mg/kg) combination treatment. The number of enriched DEGs, total number of genes comprising the biological process, and adjusted p-values are shown.

### Example 14.2: Differential Gene Expression Analysis of Select Biological Processes

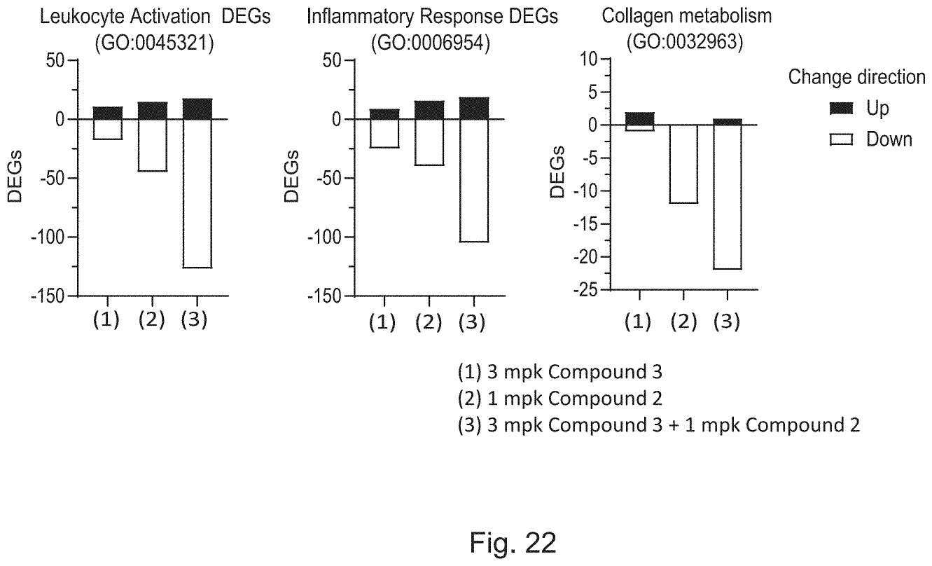

Other biological processes relevant to NASH disease were also examined. FIG. 22 shows the number of Up and Down regulated DEGs (vehicle NASH control vs. treatment) associated with different biological processes relevant to NASH and fibrosis including: leukocyte activation (GO:0045321); inflammatory response (GO:0006954), and collagen metabolic process (GO:0032963). For each biological process examined, the combination of Compound 3 with Compound 2 consistently showed greater than expected number of DEGs relative to single agent treatment groups. In addition, the combination of Compound 3 with Compound 2 showed a greater than expected number of down regulated DEGs than would have been expected based on the results of single agent treatment.

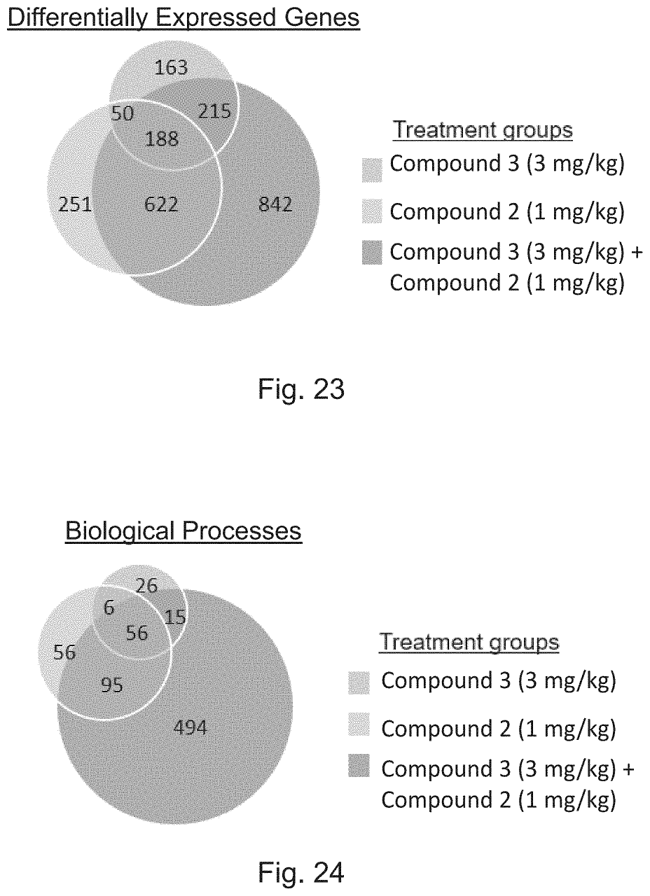

FIG. 23 shows the number and overlap of DEGs (vs. vehicle NASH control) identified in each treatment group using absolute fold-change and adjusted p-value cutoffs of ≥1.5 and <0.05, respectively. The total number of differentially expressed genes was greater than expected with Compound 3 and Compound 2 in combination, with >800 unique to the combination, and this was largely driven by a higher number of downregulated DEGs. FIG. 24 shows the number and overlap of biological processes that were significantly enriched in treatment groups relative to NASH control. An FDR-adjusted p-value of <0.05 was used as a cut-off for statistical significance.

### Example 14.3: Additional Effects on Mouse NASH Model

On day 28 of treatment as described in Example 14, animals were euthanized for sample collections. Analysis of cholesterol, triglycerides, and ALT was done using a Hitachi 7180 clinical analyzer. Liver samples were processed for lipid quantification (colorimetric assays, SpectraMax 340PC384), histology, and RNA analysis. RNAseq library preparation (n=5 per group) and sequencing was performed using Illumina standard protocols. Alignment of sequencing reads was performed using STAR aligner and read counts were estimated using RSEM. Differentially expressed genes (DEGs) relative to NASH control were determined using EdgeR. Gene ontology analysis was performed using Advaita software.

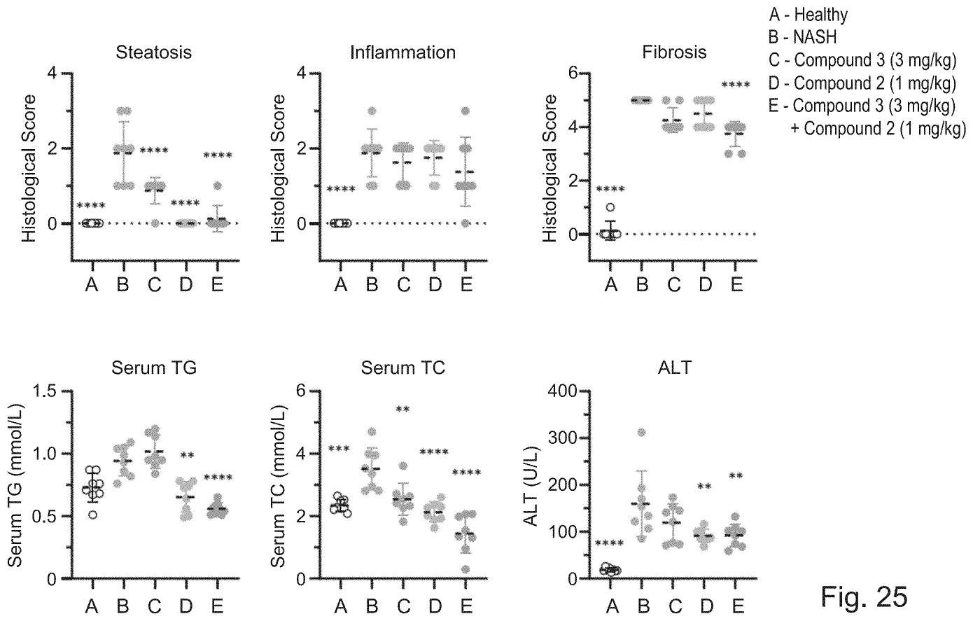

FIG. 25 shows liver steatosis, inflammation, and fibrosis as quantified by histological analysis for degree of steatosis, lobular inflammation, and fibrosis. Serum was collected at termination and analyzed for triglycerides (TG), total cholesterol (TC), and a biomarker of liver damage, alanine aminotransferase (ALT). Data for individual animals (dots) and mean (dashed line) are presented; **p<0.01, ***p<0.001, ****p<0.0001 vs NASH vehicle control (NASH). Statistics determined by one-way ANOVA followed by Tukey. The combination treatment of Compound 1 and Compound 2 significantly improved multiple components of NASH, including steatosis, fibrosis, serum triglycerides, total cholesterol, and liver damage as measured by ALT.

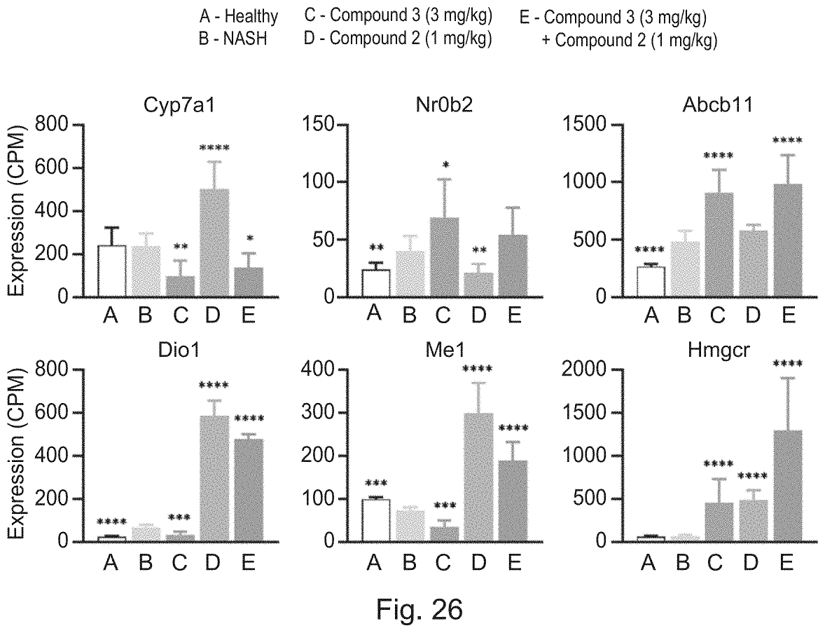

FIG. 26 shows mean expression levels of genes associated with FXR and THRβ pathway activation. FXR and THRβ pathway genes were modulated in both single and combination treatment groups.

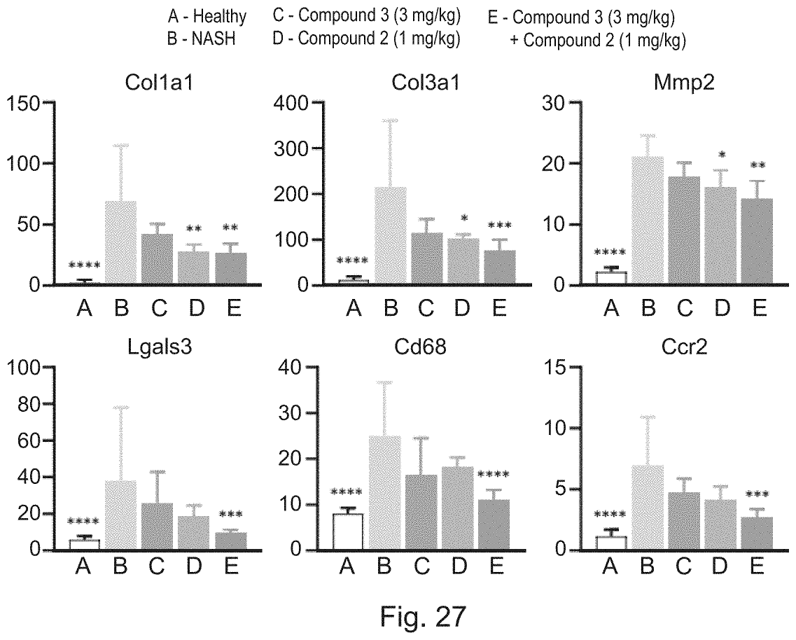

FIG. 27 shows mean expression levels (count per million reads, CPM) of genes associated with collagen/fibrosis and inflammation pathways, which were determined by RNAseq. *p<0.05, **p<0.01, ***p<0.001, ****p<0.0001 vs. vehicle (NASH) control. Error bars represent standard deviation (n=5). The combination treatment of Compound 1 and Compound 2 significantly reduced expression of collagen/fibrosis genes and inflammatory genes such as Col1a1, Col3a1, Mmp2, Lgals3, Cd68, and Ccr2.

### Conclusions

Treatment with Compound 3 and Compound 2 in combination resulted in gene expression changes that were consistent with on-target agonism of FXR and THR-β, respectively. The combination treatment of Compound 3 and Compound 2 significantly reduced expression of fibrosis and inflammatory genes.

Gene ontology enrichment analysis identified the unpredictable result that nearly 500 biological processes were uniquely enriched by Compound 3 and Compound 2 combination treatment, including down-regulation of those related to immune processes (inflammation), leukocyte function, and collagen (including collagen production) (see FIG. 22, FIG. 27). Together these data support the concept that the combination of Compound 3 and Compound 2 may provide additional benefit in NASH relative to single agent therapies, such as reducing the inflammatory component or fibrotic component of NASH more significantly than a single agent therapy alone. These affects are expected to reduce disease severity, as well as disease progression.

### Example 15: Safety, Tolerability, Efficacy of Combination Therapy in Patients with NASH

A randomized, double-blind, placebo-controlled study is conducted to evaluate the safety and efficacy of combination treatments, for example, Compound 1 and Compound 2, or the combination of Compounds 1, 2, and 3. Subjects with NASH are treated once daily with the SSAO inhibitor and the THR-β agonist in combination for between about 12 to 52 weeks. Liver fat is monitored by MRI-PDFF, fibro-inflammation is monitored by corrected T1 (cT1) relaxation time, and serum-based non-invasive fibrosis or NASH markers such as C3, TIMP-1, PIIINP, CK-18, and ALT, are measured. Changes in serum lipid levels, such as LDL-c and other lipids, are also monitored.

### Example 16: Production of Potassium Salt of Compound

2Ethyl (E)-(2-cyano-2-(2-(3,5-dichloro-4-((4-oxo-3,4-dihydrophthalazin-1-yl)oxy)phenyl)hydrazineylidene)acetyl)carbamate (7.4 kg, 0.99-1.01×), potassium acetate (7.4 kg, 0.95-1.00×) and DMAc (41 kg, 5.6-6.0×) were charged into a 500 L GL reactor. The resulting mixture was kept at 80-90° C. for 12-16 h. The mixture was adjusted to 20-30° C. A solution of KOH (0.85 kg, 0.11-0.17×) in process water (8 kg, 1.0-1.5×) was added at 20-30° C. for 1-2 h. The mixture was stirred at 20-30° C. for 1-2 h. Process water (39 kg, 5.0-5.5×) was added at 20-30° C. for 4-6 h. The mixture was stirred at 20-30° C. for 2-3 h. The resulting mixture was centrifuged by a stainless-steel centrifuge. The wet cake was rinsed with process water twice (18+20 kg, 2-3×). Charged the wet cake and process water (38 kg, 5.0-6.0×) into the 500 L GL reactor. The mixture was stirred at 20-30° C. for 2-3 h. The resulting mixture was centrifuged by a stainless-steel centrifuge. The wet cake was rinsed with process water twice (18+22 kg, 2-3×), and dried by a stainless steel dryer under reduced pressure at 55-65° C. to obtain the crude potassium salt of Compound 2 (5.45 kg, purity: 98.4%; assay: 95.9%; yield: 72%).

The crude potassium salt of Compound 2 (5.3 kg, 0.99-1.01×) and DMSO (43 kg, 6.0-8.0×) were charged into a 250 GL reactor (R1). The resulting mixture was kept at 40-50° C. for 0.5-1 h to give a clear solution. Ethyl acetate (EA) (22 kg, 4.0-4.5×) was added at 40-50° C. over 1-2 h. The resulting mixture was filtered through inline filter and transferred into a 250 GL reactor (R₂). R1 was rinsed with DMSO (1.8 kg, 0.2-0.5×), and the material was filtered through inline filter and transferred into R2. Adjusted to 40-50° C. and charged EA (22 kg, 4.0-4.5×) into R2 at 40-50° C. over 1-2 h. Charged Compound 2 seeds (0.010 kg, 0.001-0.002×) into R2. The resulting mixture was stirred at 40-50° C. for 1-2 h. Charged EA (67 kg, 12.0-13.0×) into R2 at 40-50° C. over 12-15 h, and stirred at 40-50° C. for 2-3 h. Charged EA (27 kg, 1.0-5.0×) into R2 at 40-50° C. over 2 h, and stirred at 40-50° C. for 2-3 h. Adjusted R2 to 20-30° C. over 2 h and stirred at 20-30° C. for 4-6 h. The resulting mixture was filtered in a stainless steel filter dryer. The wet cake was rinsed with EA twice (16+14 kg, 2-3×). Charged EA (36 kg, 6-7×) into the stainless steel filter dryer. Adjusted to 20-30° C. and stirred at 20-30° C. for 2-3 h. The resulting mixture was filtered in a stainless steel filter dryer. The wet cake was rinsed with EA (16 kg, 2-3×), and dried by a stainless steel filter dryer under reduced pressure at 60-70° C. The material was sieved to give the purified potassium salt of Compound 2 (4.16 kg, purity: 99.81%; assay: 97.6%; yield: 80%).

### Example 17

Methods

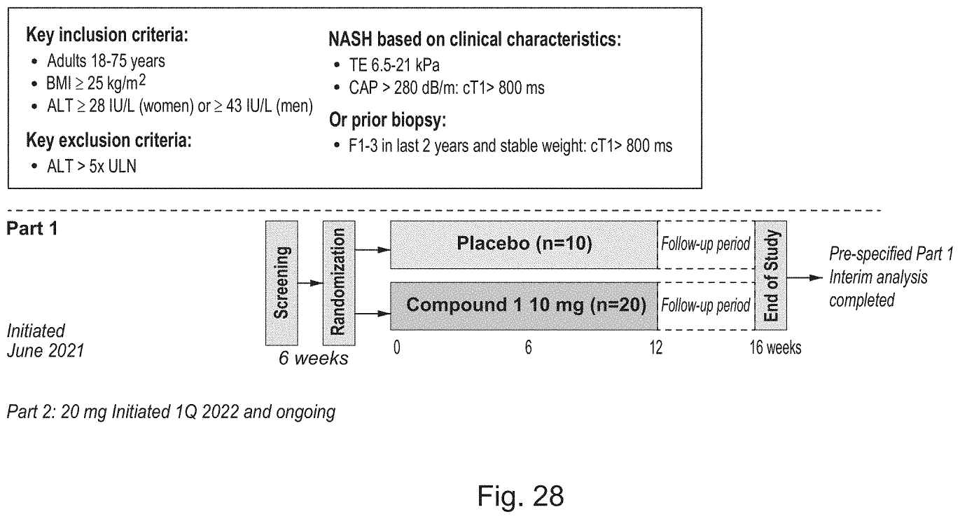

Part 1 of the two-part trial was a double-blind, placebo-controlled study in 30 adults with non-cirrhotic NASH phenotype evaluating 10 mg Compound 1 once daily (QD) for 12 weeks followed by off-treatment evaluation at week 16 (FIG. 28). Part 1 interim analysis primary endpoint was safety assessed by adverse events (AEs) and laboratory tests; percent change from baseline (BL) in plasma VAP-1 activity was a secondary endpoint. Exploratory imaging and blood-based biomarkers of liver inflammation and fibrosis were also assessed. Analyses of change (or percent change) from baseline used an ANCOVA model with change (or percent change) from baseline as the dependent variable including treatment group and randomization strata as fixed effects and baseline as a covariate.

Results

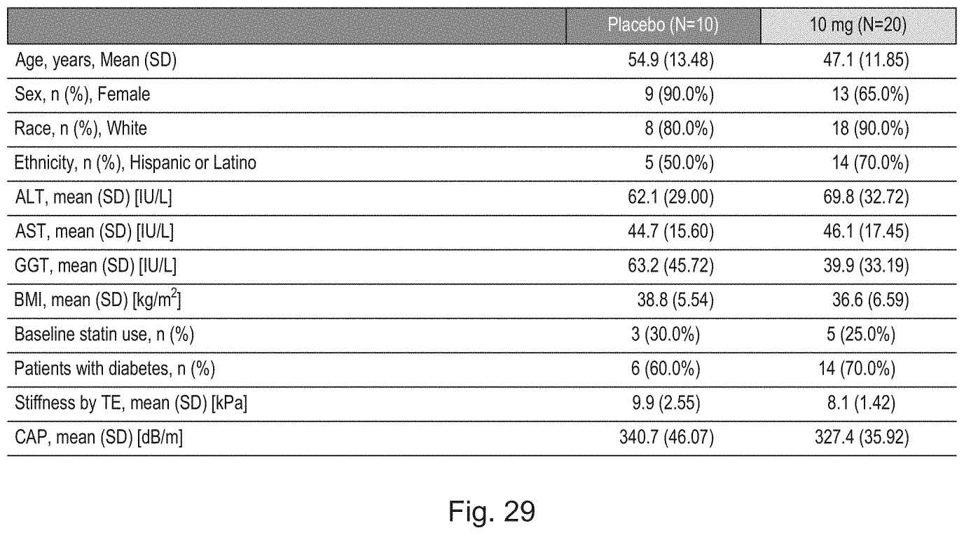

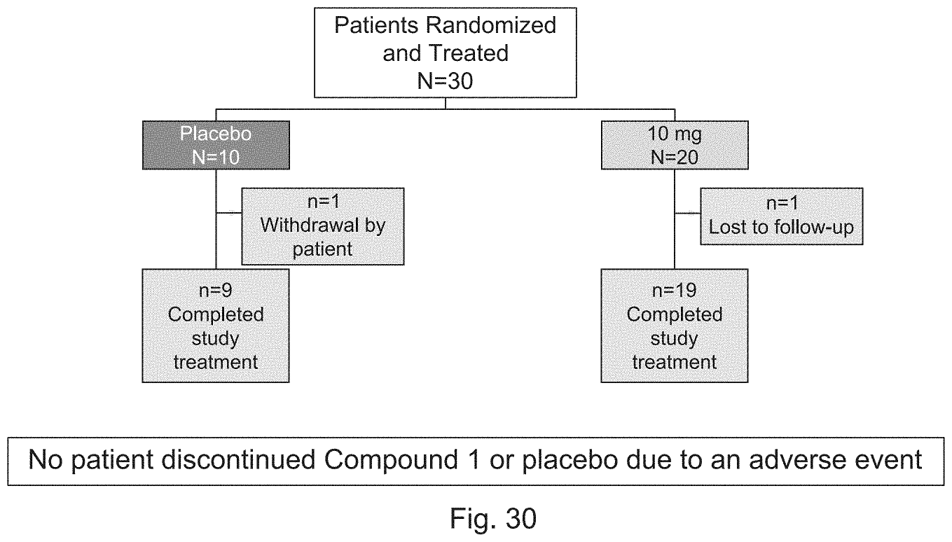

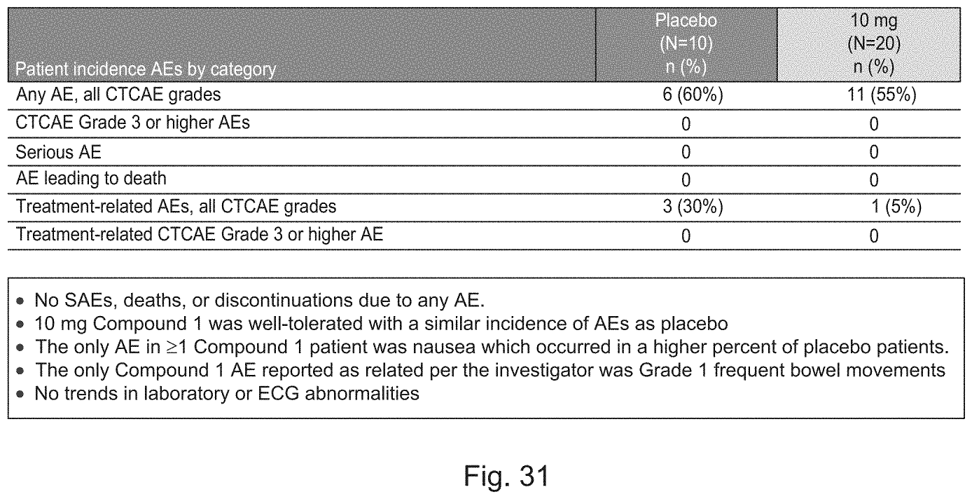

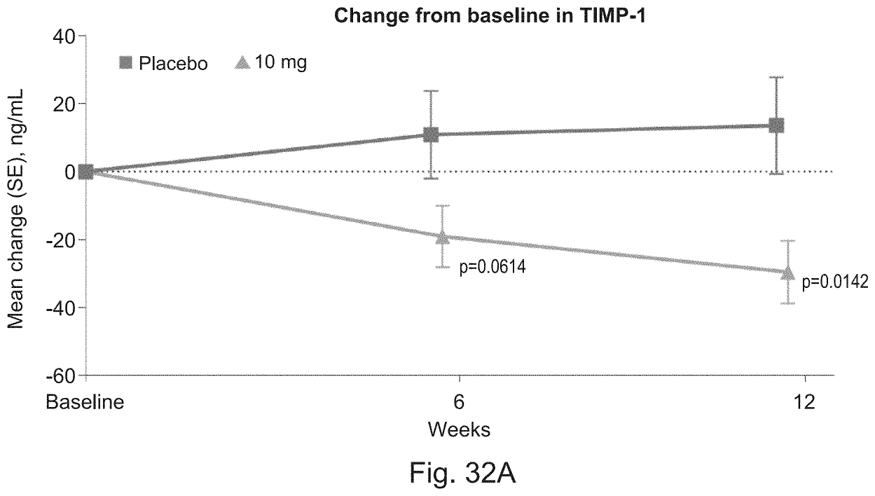

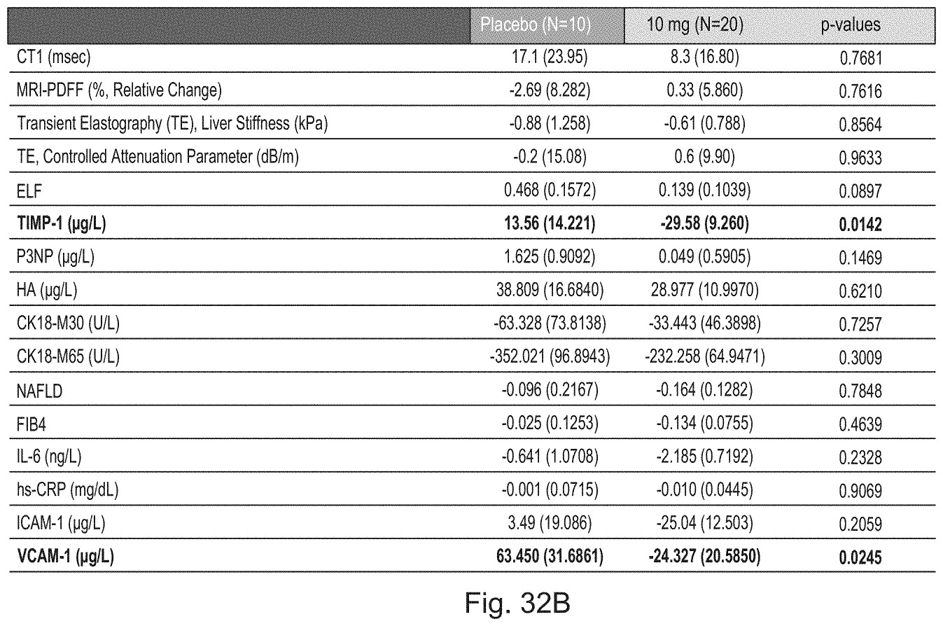

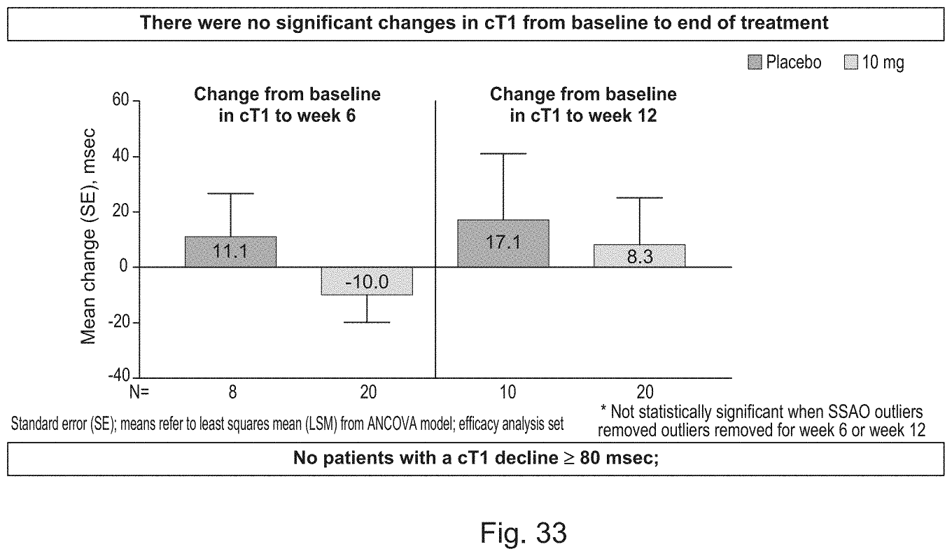

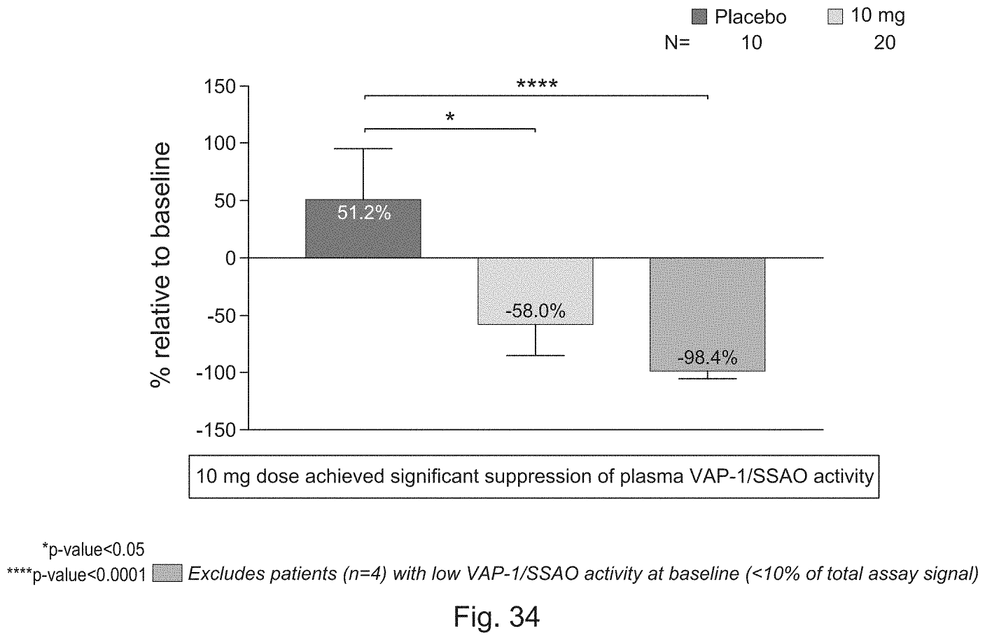

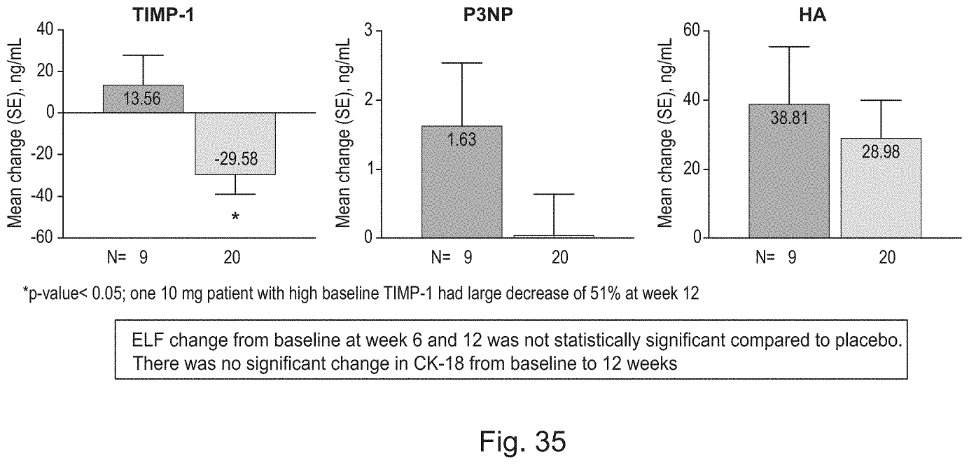

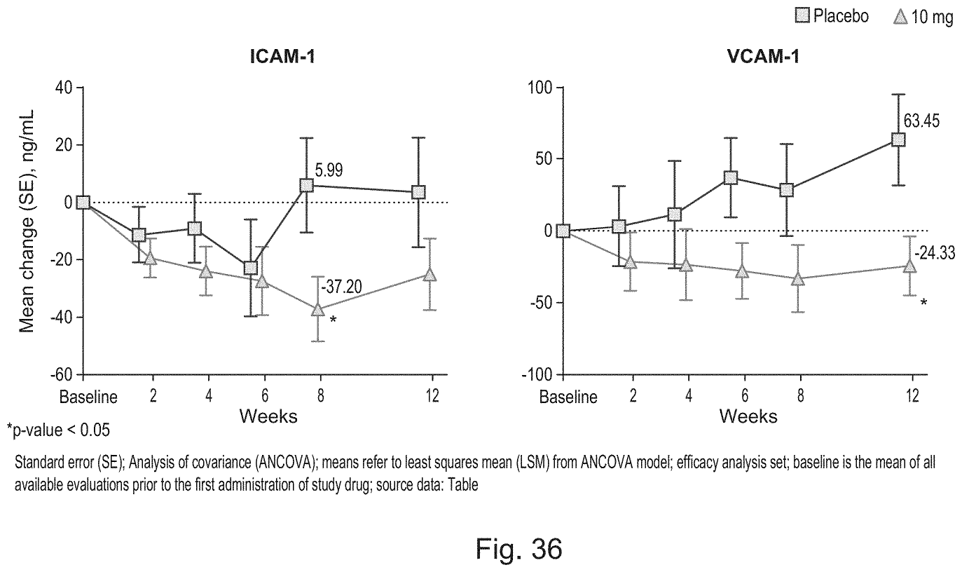

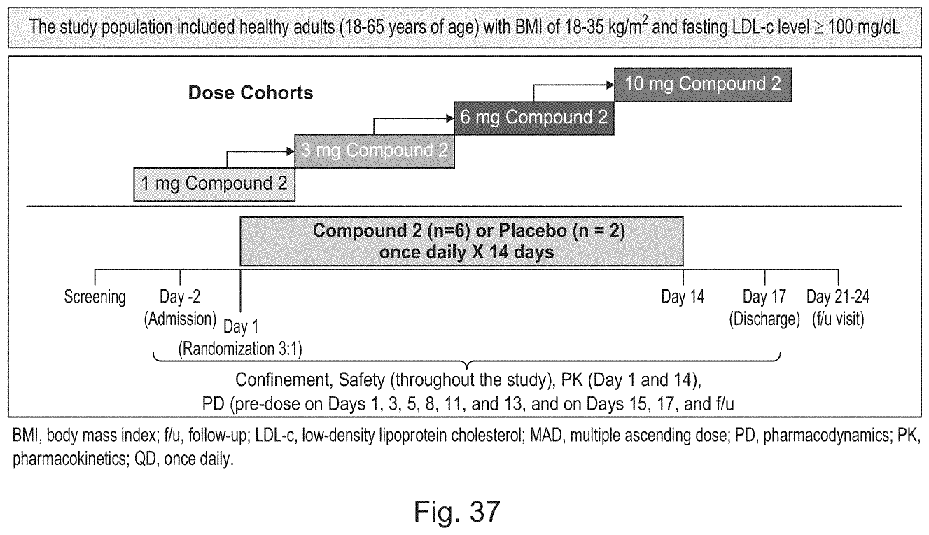

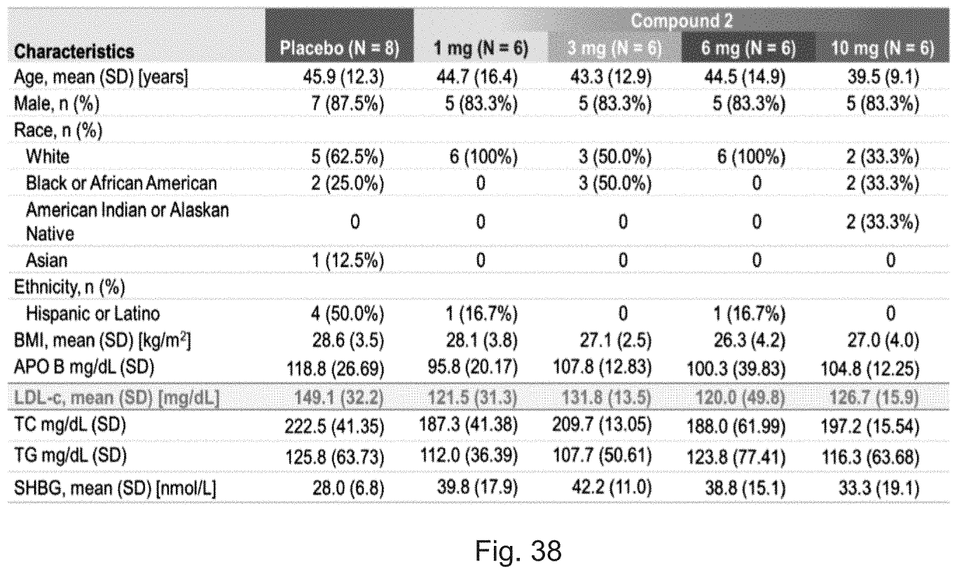

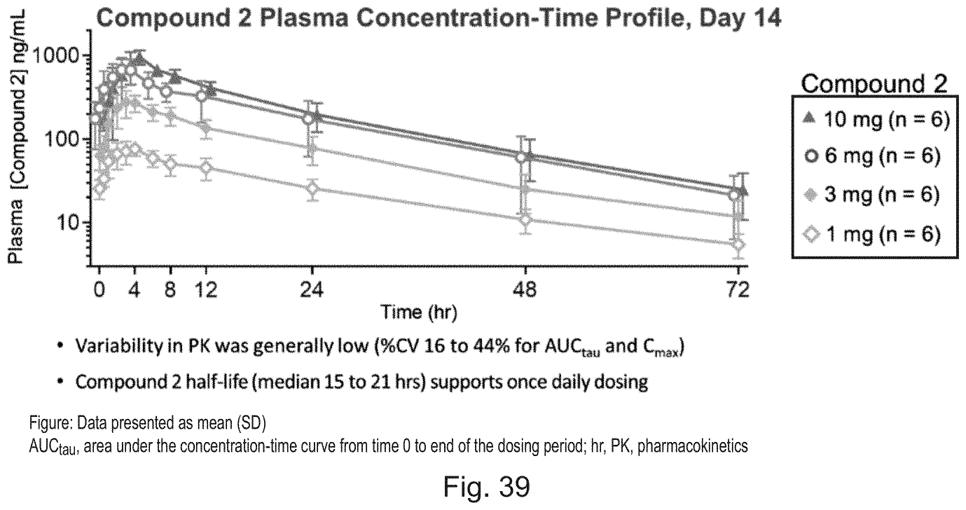

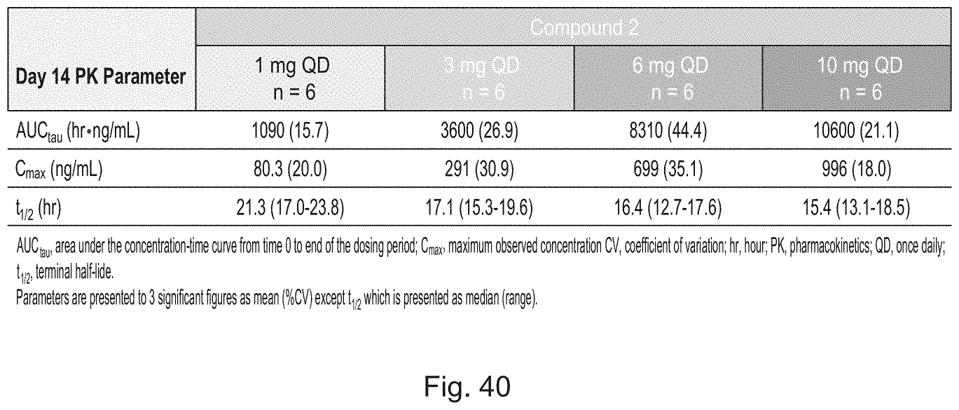

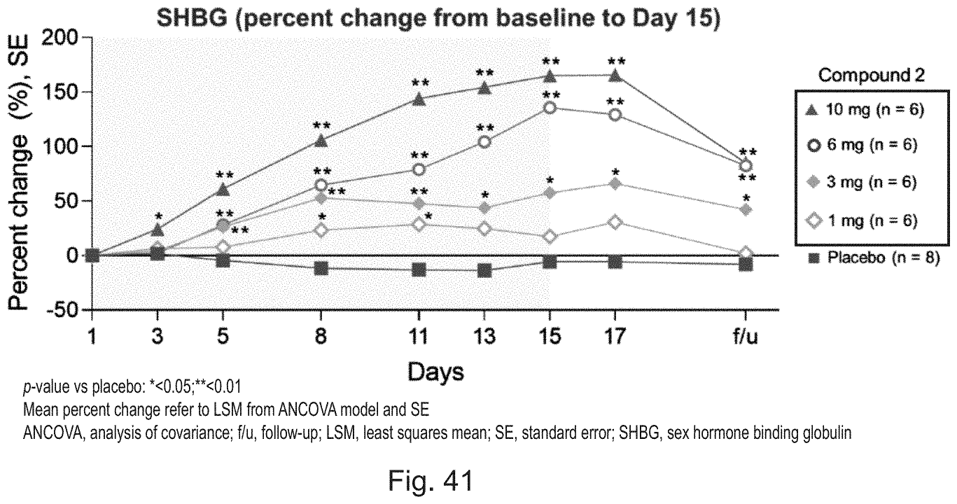

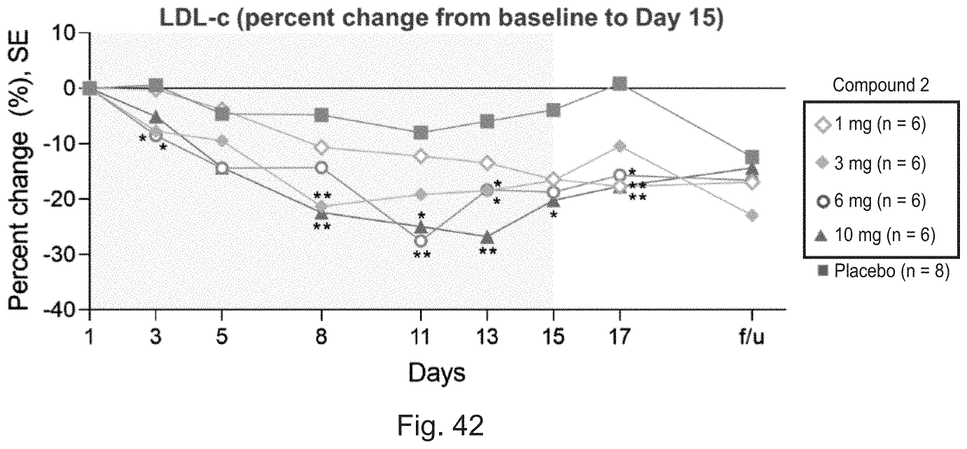

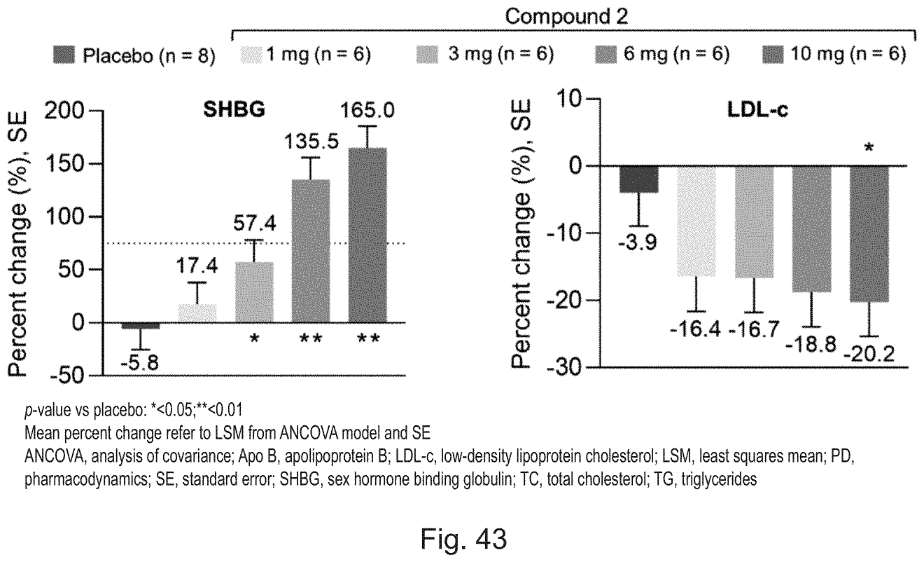

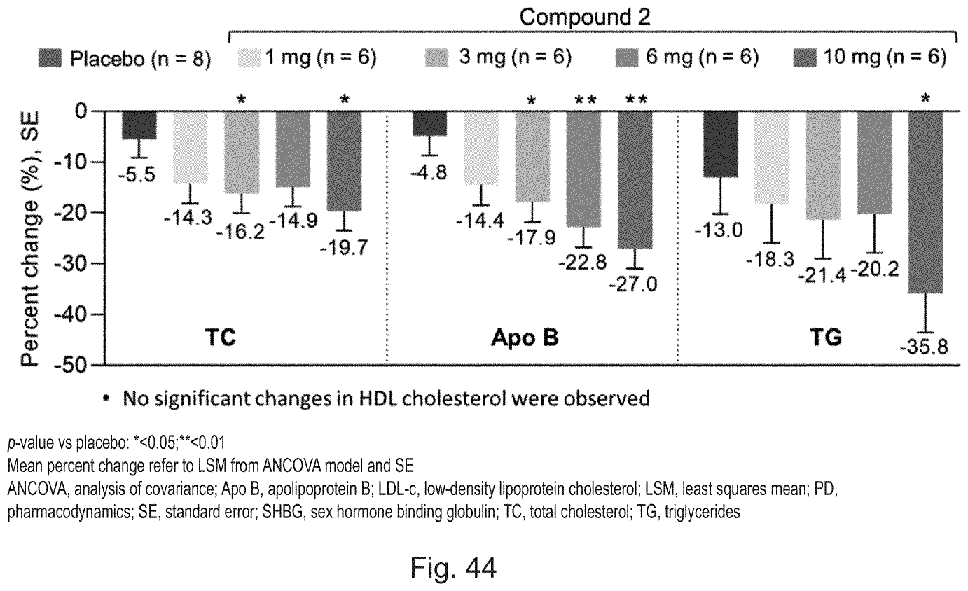

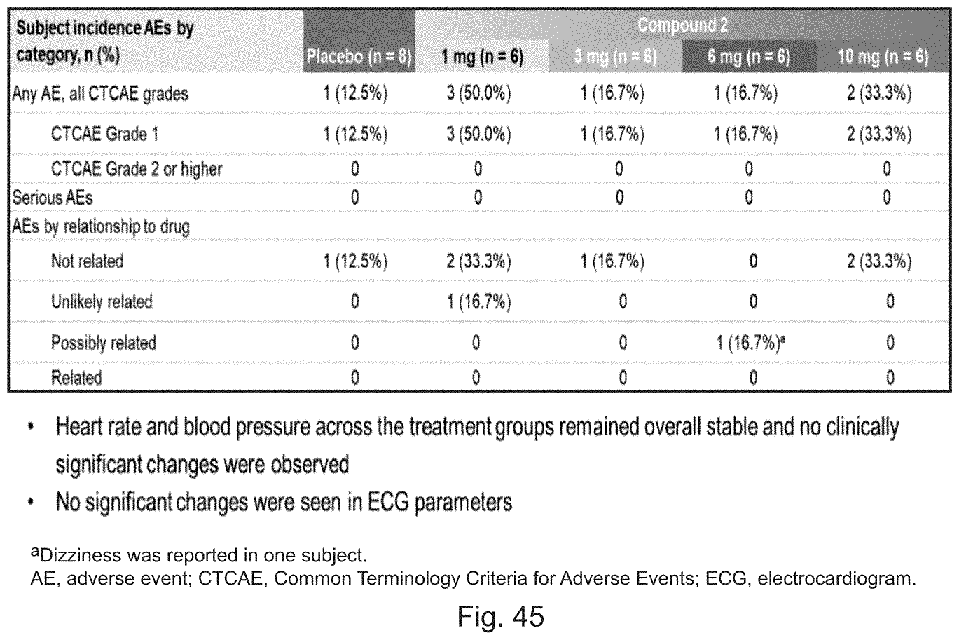

The demographics and baseline characteristics of patients in the placebo and treatment groups were overall similar (FIG. 29). Nearly all patients (28/30, 95% of Compound 1 and 90% of placebo) completed Part 1 (FIG. 30). Compound 1 (10 mg) was well-tolerated with a similar incidence of AEs as placebo (FIG. 31). All AEs were mild or moderate with no serious AEs or trends in AEs or laboratory abnormalities reported (FIG. 32A and FIG. 32B). There were no significant changes in cT1 from baseline to end of treatment (FIG. 33). Compound 1 (10 mg) resulted in >98% inhibition of plasma VAP-1 activity in most subjects by week 2 and sustained suppression through week 12 (FIG. 34). There were no statistically significant differences between placebo and 10 mg Compound 1 in change from BL cT1, liver fat fraction, liver enzymes, or cytokeratin-18. At Week 12, significant differences from placebo in a marker of cell adhesion, VCAM-1, and a marker of hepatic fibrogenesis, tissue inhibitor of metalloproteinase-1 (TIMP-1) were observed (FIGS. 35 and 36). Markers of cell adhesion, including VCAM-1 and ICAM-1: VCAM-1, decreased by 24.327 (20.5850) μg/L, in the Compound 1 group and increased by a mean (±SE) of 63.450 (31.6861) μg/L, in the placebo group relative to BL (p=0.0245) at Week 12. ICAM-1 decreased by a mean (±SE) of 34.85 (56.03) μg/L, in the Compound 1 group and increased by a mean (±SE) of 7.46 (46.97) μg/L in the placebo group relative to BL (p=0.0313) at Week 8. At Week 12, TIMP-1 decreased by a mean (±SE) of 29.58 (9.26) ng/mL in the Compound 1 group and increased by a mean (±SE) of 13.56 (14.22) ng/mL in the placebo group relative to BL (p<0.05).

### Conclusions

Compound 1 was well-tolerated with a safety profile similar to placebo in patients with baseline multiparametric MRI and LS values indicative of NASH with at least stage 2 fibrosis. Compound 1 (10 mg) led to near complete inhibition of plasma VAP-1 activity, decreased levels of the hepatic fibrogenesis marker TIMP-1, and statistically significant decrease in the cell adhesion biomarkers, ICAM-1 (at Week 8) and VCAM-1 (at Week 12), compared to placebo. No statistically significant differences were observed between Compound 1 and placebo on other markers of liver inflammation and injury following 12 weeks of treatment.

### Example 18. Clinical Evaluation of Compound 2 in Healthy Subjects with Elevated LDL-c

Objectives: Assess the overall safety and tolerability of multiple ascending doses of Compound 2 in healthy subjects with elevated LDL-c.

Secondary Objectives: Evaluate the PK and PD of Compound 2 in healthy subjects with elevated LDL-c following multiple ascending doses of Compound 2,

Primary Endpoints: Treatment-emergent adverse events (TEAEs), vital signs, clinical laboratory parameters, and electrocardiogram (ECG) monitoring

Secondary Endpoints: Plasma PK parameters for Compound 2, PD markers of THR-β agonist target engagement including LDL-c and other lipid parameters and sex hormone binding globulin (SHBG)

Healthy volunteers with mildly elevated LDL-c were randomized 3:1 to Compound 2 (n=6) or placebo (n=2). Volunteers randomized to Compound 2 received multiple doses of 1, 3, 6 or 10 mg of Compound 2 once daily for 14 days in the MAD cohort of the study.

Results

Compound 2 was generally safe and well-tolerated with a similar incidence of AEs across all Compound 2 treatment groups and placebo. All AEs were mild to moderate with no apparent dose relationship. One placebo subject (1 mg cohort) terminated study early due to withdrawal of consent; all Compound 2 subjects completed study with no premature discontinuations. No study stopping criteria or dose escalation stopping criteria were met.

Liver biochemistry: ALT, AST, ALP and total bilirubin values were overall similar across the treatment groups. No subject receiving Compound 2 had ALT increase to ≥2×ULN. No evidence of DILI. Thyroid hormone: No symptoms of hyper/hypothyroidism. Mean TSH and free T3 values were highly variable but generally similar across the groups. Dose-dependent declines of free T4 were observed among Compound 2 groups consistent with peripheral thyroid hormone modulation observed with other THR-β agonists. Other laboratory assessments (e.g., clinical chemistry, hematology) showed no apparent trends.

Once daily dosing of Compound 2 at 1, 3, 6, and 10 mg for 14 days was overall safe and well-tolerated with no clinical signs or symptoms of hypo/hyperthyroidism or THR-α agonism. Compound 2 exhibited dose-proportional PK with low variability and a half-life suitable for once daily dosing. Compound 2 increased SHBG, a key marker of hepatic THR-β engagement, in a dose-dependent manner. Compound 2 led to significant decreases in circulating atherogenic lipid levels including LDL-c, Apo B, total cholesterol, and triglycerides. Taken together, PD data indicate that administration of Compound 2 led to robust THR-β target engagement in the liver.

All publications, including patents, patent applications, and scientific articles, mentioned in this specification are herein incorporated by reference in their entirety for all purposes to the same extent as if each individual publication, including patent, patent application, or scientific article, were specifically and individually indicated to be incorporated by reference.

Although the foregoing invention has been described in some detail by way of illustration and example for purposes of clarity of understanding, it is apparent to those skilled in the art that certain minor changes and modifications will be practiced in light of the above teaching. Therefore, the description and examples should not be construed as limiting the scope of the invention.

### Example 19: Effect of 12 Weeks of Mono- and Combination-Treatments with Compound 3 and Compound 2 on Metabolic Parameters, Hepatic Pathology and NAFLD Activity Score Including Fibrosis Stage in Male Biopsy-Confirmed DIO-NASH Mice

### LIST OF ABBREVIATIONS

Acta2 Actin alpha 2 smooth actin
ALP Alkaline phosphatase
ALT Alanine transaminase
ANOVA Analysis of Variation
AST Aspartate transaminase
BW Body weight
CD146 Melanoma cell adhesion molecule
cDNA Complementary DNA
CK18 M30 Cytokeratin 18 M30
Col1a1 Collagen type I alpha 1
DAB 3,3′-Diaminobenzidine
DEG Differentially Expressed Gene
Dhcr7 7-dehydrocholesterol
DIO Diet-induced obesity
DIO-GAN Diet-induced obesity Gubra Amylin NASH model
ELISA Enzyme-linked immunosorbent assay
FFPE Formalin-fixed paraffin-embedded
FPKM Fragments Per Kilobase of transcript per Million mapped reads
FXR Farnesoid X Receptor
g g-force
Gal-3 Galectin-3
GAN Gubra Amylin NASH
H&E Hematoxylin and Eosin staining
HDL-c High-density lipoprotein cholesterol
Hmgcs1 Hydroxymethylgluteraryl-CoA synthase
HPMC hydroxypropyl methylcellulose
HRP Horseradish peroxidase
IHC Immunohistochemistry
kg Kilogram
LDL-c Low-density lipoprotein cholesterol
Lgals3 Galectin-3
mg Milligram
NA Not applicable
NAFLD Nonalcoholic Fatty Liver Disease
NAS NAFLD Activity Score
NASH Nonalcoholic steatohepatitis
NP-40 Nonidet P-40
PO Per os (oral dosing)
PSR Picrosirius red
QD Once daily

### INTRODUCTION

Nonalcoholic steatohepatitis (NASH), a disease manifested by hepatic inflammation and injury in the context of liver steatosis, will likely require combination therapy targeting multiple aspects of the disease to achieve high levels of disease resolution. Small molecule agonists of Farnesoid X Receptor (FXR), a nuclear hormone receptor that maintains homeostasis of metabolic pathways, and thyroid hormone receptor beta (THR-β), a nuclear hormone receptor that regulates metabolic pathways complementary to FXR, are in development for the treatment of NASH. Compound 3, a non-steroidal agonist of FXR, and Compound 2, a liver distributed, selective agonist of THR-β, were evaluated alone and in combination in a diet-induced mouse model of NASH.

Materials and Methods

Animal Handling and Study Design

C57BL/6JRj male mice (n=138) were fed the Gubra Amylin NASH (GAN) diet (40% fat, 22% fructose, 2% cholesterol [D09100310, Research Diets]) or lean chow diet for 35 weeks before treatment start. Prior to treatment all animals underwent liver biopsy for histological confirmation (steatosis score ≥2 and fibrosis stage ≥1) and stratification using the nonalcoholic fatty liver disease (NAFLD) activity scoring (NAS) and fibrosis staging system. The diet-induced obese mice on GAN diet (DIO-GAN) were randomized to 8 treatment groups (Error! Reference source not found.) based on percent-area of picrosirius red (PSR) staining. DIO-GAN mice (n=16 per group) received treatment (PO, QD) for 12 weeks with vehicle (0.5% HPMC+0.2% Tween-80 in Tris buffer [50 mM, pH 8]), Compound 3 (10 mg/kg), Compound 2 (0.3 mg/kg [low], 2 mg/kg [med], or 10 mg/kg [high]), or combination treatments of Compound 3 with Compound 2 (Combo-low, Combo-med or Combo-high). Vehicle-dosed lean chow-fed controls served as healthy controls (n=10). Mice were maintained on their respective diets (GAN or lean chow) for the duration of the study. Within-subject comparisons (pre- vs. post-treatment) were performed for liver biopsy histopathological scores. Terminal quantitative endpoints included plasma/liver biochemistry, liver histomorphometry, and liver transcriptomic analysis by RNAseq.

TABLE 25
Treatment Groups

| | | |
|---|---|---|
| Group | Dose | N |

| | | |
|---|---|---|
| Lean Chow vehicle control | NA | 10 |
| DIO-GAN vehicle control | NA | 16 |

| | | | |
|---|---|---|---|
| Compound 3 | 10 | mg/kg | 16 |
| Compound 2-low | 0.3 | mg/kg | 16 |
| Compound 2-med | 2 | mg/kg | 16 |
| Compound 2-high | 10 | mg/kg | 16 |

| | | |
|---|---|---|
| Combo-low | 10 mg/kg Compound 3 + | 16 |
| | 0.3 mg/kg Compound 2 | |
| Combo-med | 10 mg/kg Compound 3 + | 16 |
| | 2 mg/kg Compound 2 | |
| Combo-high | 10 mg/kg Compound 3 + | 16 |
| | 10 mg/kg Compound 2 | |

Treatment groups assigned to study. DIO-GAN vehicle controls administered vehicle (0.5% HPMC + 0.2% Tween 80 in Tris buffer [pH 8]) once daily.
NA, not applicable.

Liver Biopsy Processing and Scoring

Formalin-fixed paraffin-embedded (FFPE) liver biopsies were prepared by placing liver samples into 10% neutral buffered formalin for ˜24 hours and then transferred to 70% ethanol prior to storage at 4 C. FFPE were placed in the Histokinette to infiltrate prior to embedding in blocks. Biopsy tissues were then cut at 3 μm using a microtome and sections were mounted on slides. Liver sections were stained with Hematoxylin and Eosin (H&E) to assess steatosis, inflammation, and ballooning, and PSR to assess fibrosis. Additionally, slides were processed to detect type I collagen (Col1a1), galectin-3 (Gal-3), and smooth muscle actin (α-SMA) protein expression by immunohistochemistry (IHC). For H&E staining, slides were incubated in Mayer's Hematoxylin (Dako), washed with tap water, stained in Eosin Y solution (Sigma-Aldrich), dehydrated, and coverslipped. For PSR, slides were incubated in Weigert's iron hematoxylin (Sigma-Aldrich), washed in tap water, stained in Picro-sirius red (Sigma-Aldrich), and washed twice in acidified water. Excess water was removed by shaking the slides and the slides were then dehydrated with ethanol, cleared in xylene, and cover-slipped. The NAS and fibrosis stage were scored as described previously (Kleiner et al. 2005). NAS represents the unweighted sum of steatosis, inflammation, and ballooning scores and ranges for 0-8 (Table 26); fibrosis stage ranges from 0 (no fibrosis) to 4 (cirrhosis). For detection of Col1a1, Gal-3 and α-SMA, IHC was performed by standard procedures. Briefly, after antigen retrieval and blocking of endogenous peroxidase activity, slides were incubated with primary antibody (Col1a1: Southern Biotech, Cat. 1310-01; Gal-3: Biolegend, Cat. 125402; α-SMA: Abcam Cat. Ab124964). Primary antibody was detected using a polymeric HRP-linker antibody conjugate. Primary antibody was visualized with DAB as chromogen. Finally, sections were counterstained in hematoxylin and cover-slipped.

Table 26: Histological Scoring of NAS and Fibrosis

| Feature | Degree | Score |
|---|---|---|
| Steatosis | <5% | 0 |
| (Percentage of hepatocytes | 5-33% | 1 |
| with lipid droplets) | >33-66% | 2 |
| | >66% | 3 |
| Lobular inflammation | No foci | 0 |
| | <2 foci | 1 |
| | 2-4 foci | 2 |
| Ballooning degeneration | None | 0 |
| | Few | 1 |
| | Many cells/prominent ballooning | 2 |
| Fibrosis | None | 0 |
| | Perisinusoidal or periportal | 1 |
| | Perisinusoidal & | 2 |
| | portal/periportal | 3 |
| | Bridging fibrosis Cirrhosis | 4 |

Analysis of Liver Enzymes, Plasma Lipids and CK18 M30

Terminal blood was harvested by cardiac puncture from mice anesthetized with isoflurane (2-3%), mixed with anticoagulant, and placed at 4 C prior to centrifugation at 3000×g for 10 minutes. Plasma supernatants were transferred to new tubes and immediately frozen on dry ice and stored at −80 C. Alanine transaminase (ALT), Aspartate transaminase (AST), Alkaline phosphatase (ALP), Triglycerides (TGs), Total Cholesterol (TC), High-density lipoprotein (HDL-c), and Low-density lipoprotein (LDL-c) were measured using commercial kits (Roche Diagnostics) on the Cobas c 501 autoanalyzer according to manufacturer's instructions. Cytokeratin 18 M30 (CK18 M30) was measured from plasma using a commercial ELISA kit (Cusabio) according to the manufacturer's instructions.

Analysis of Liver Lipids

Liver Samples were Homogenized and TGs and TC was Extracted in 5% NP-40 by heating (2×) at 90 C. Samples were centrifuged and the TG and TC content was measured in the supernatant using commercial kits (Roche Diagnostics) on the Cobas c 501 autoanalyzer, according to the manufacturer's instructions.

Liver Transcriptomic Analysis by RNAseq

Tissue was collected and snap-frozen in liquid nitrogen and stored at −80 C until processing. RNA was isolated using a NucleoSpin kit (MACHEREY-NAGEL). A total of 10 ng to 1 μg purified RNA from each sample was used to generate cDNA libraries using the NEBNext Utra II Directional RNA Library Prep Kit for Illumina (New England Biolabs). cDNA libraries were then sequenced on a NextSeq 500 using NextSeq 500/550 High Output Kit V2 (Illumina). Sequencing data was aligned to the mouse genome using the Spliced Transcripts Alignment to a Reference (STAR) software. Differentially expressed genes were identified using the R-package DESeq2.

Analysis of Trough Plasma Compound Levels

Terminal plasma samples were harvested by cardiac puncture approximately 21-24 hours after the last administration of compound(s). Terminal plasma samples were analyzed by high resolution LC-MS/MS using a Triple Quad 6500+ instrument. 20 μL of plasma sample was mixed with 200 μL of internal standard solution (100 ng/mL Labetalol+100 ng/mL Tolbutamide in acetonitrile), vortexed, and centrifuged at 4,000 rpm for 15 min at 4° C. Supernatant (100 μL) was transferred to a sample plate, mixed with water (100 μL), and shaken (800 rpm) for 10 min prior to injection (2 μL) onto a 1.7 μm, 2.1×50 mm ACQUITY UPLC BEH C₁₈ column (Waters) using a gradient (Table 27) mobile phase of 0.1% formic acid in water (Mobile phase A) and 0.1% formic acid in acetonitrile (Mobile phase B) at a flow rate of 0.6 mL/min. The internal standard 1 (retention time: 0.87 min) and standard 2 (retention time: 0.96 min) were used for quantification of Compound 3 (retention time: 0.87 min) and Compound 2 (retention time: 0.97 min), respectively. The internal standard (retention time: 0.76 min), was used for quantification of the glucuronide metabolite of Compound 3 (retention time: 0.77 min). Calibration curves (1-3000 ng/mL) were generated in a matrix of mouse plasma (pooled vehicle control) for each analyte.

TABLE 27
LC-MS/MS Gradient

| Time (min) | Mobile phase B (%) |
|---|---|
| Initial | 35 |
| 1.00 | 98 |
| 1.70 | 98 |
| 1.71 | 35 |
| 2.20 | Stop |

Results

Study Design Overview

The diet-induced obese, Gubra Amylin NASH model (DIO-GAN) recapitulates many of the histopathological features of human NASH (Hansen 2020). The DIO-GAN model was used to assess the efficacy of the FXR agonist, Compound 3, and the THR-β agonist, Compound 2, as single agents and in combination. To induce NASH disease, C57BL/6JRj mice were maintained a on diet high in fat, cholesterol, and fructose (GAN diet) for >35 weeks. Prior to therapeutic intervention, mice were biopsied to assess NASH disease and fibrosis severity; mice with a steatosis score <2 and fibrosis stage <1 were excluded from the study. DIO-GAN mice were then randomized into 8 treatment groups (n=16 per group) based on the percent fractional area of Picrosirius red (PSR) staining of the pre-treatment biopsy as well as body fat tissue mass determined by whole body Echo-magnetic resonance imaging (EchoMRI). In the single agent treatment arms, Compound 3 was administered by oral gavage once daily at a dose of 10 mg/kg, while Compound 2 was administered by oral gavage once daily at dose levels of 0.3 (Compound 2-low), 2 (Compound 2-med), or 10 (Compound 2-high) mg/kg. In the combination treatment arms, a constant dose level of Compound 3 (10 mg/kg) was combined with the low, med, and high doses of Compound 2 (i.e., Combo-low, Combo-med, Combo-high, respectively). DIO-GAN mice administered vehicle by oral gavage once daily served as a control. Mice were treated for a total of 12 weeks and were maintained on GAN diet throughout the study. Lean mice (n=10), maintained on normal chow diet throughout the study served as healthy controls.

Effects of Treatment on Body Weight, Food Intake, and Liver Weight

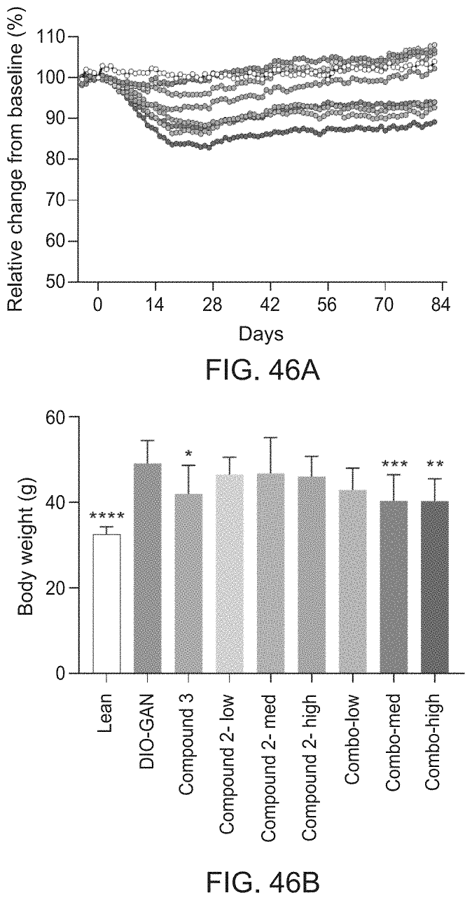

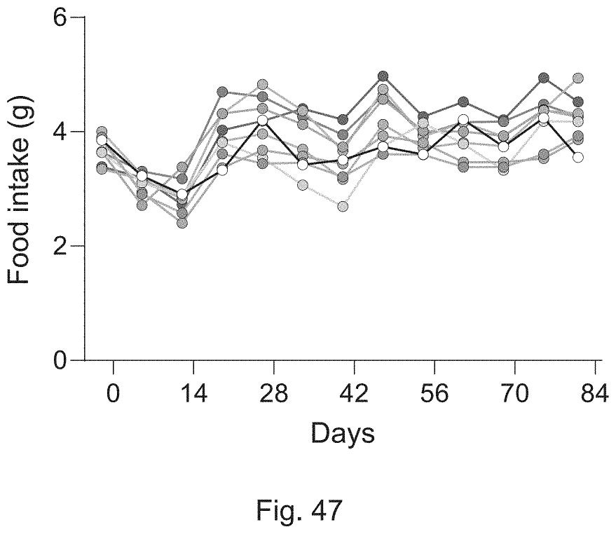

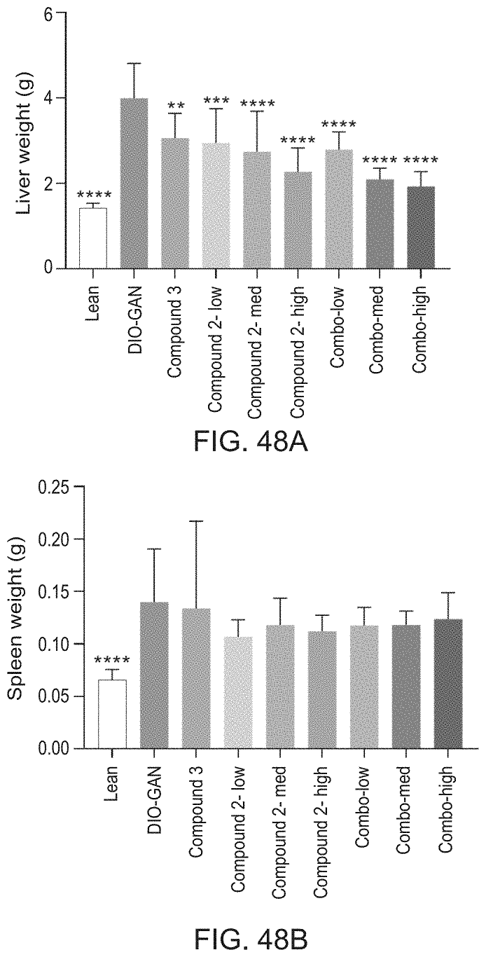

Treatment with Compound 3 alone and in combination with Compound 2 (Combo-low, Combo-med and Combo-high doses) decreased body weight during the study (FIG. 46A). At the end of study, Compound 3, Combo-med and Combo-high treatment groups were significantly lower than the DIO-GAN vehicle control (FIG. 46B). Decreases in body weight did not appear to be associated with decreases in food intake (FIG. 47). All treatment groups significantly improved hepatomegaly (FIG. 48A), with the greatest reductions in liver weight observed in the combination treatment groups (Combo-med and Combo-high); changes in spleen weight were not significant (FIG. 48B).

Effect of Treatment on Body Mass Composition

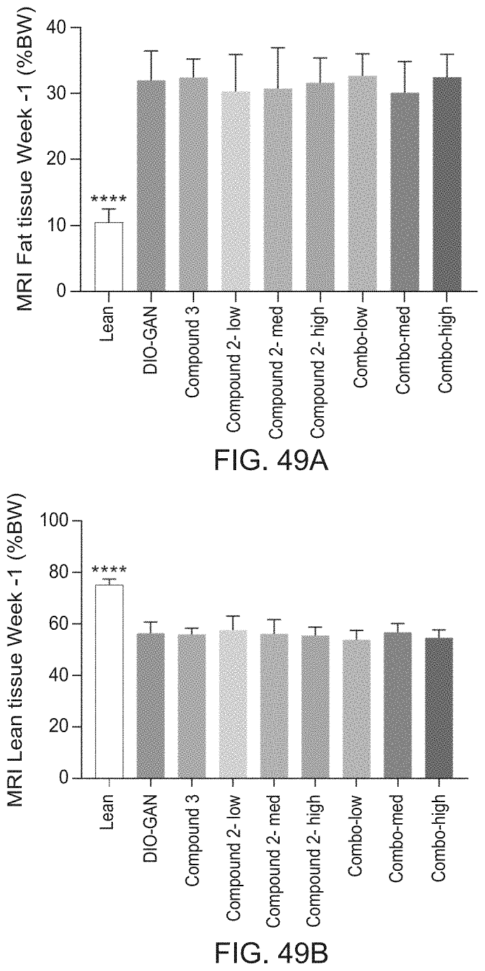

Body composition was determined at baseline (Week −1) and Week 11 of the study by whole body EchoMRI to determine the relative levels lean and fat tissue as a percentage of body weight. Baseline levels of lean and fat tissue were well balanced across treatment groups (FIG. 49A and FIG. 49B). Treatment with Compound 3 and in combination with Compound 2 reduced levels of fat tissue at Week 11 (FIG. 50A); significant increases in relative lean tissue mass were observed in the Compound 3 and combination treatment groups (FIG. 50B).

Effect of Treatment on Plasma and Liver Lipid Levels

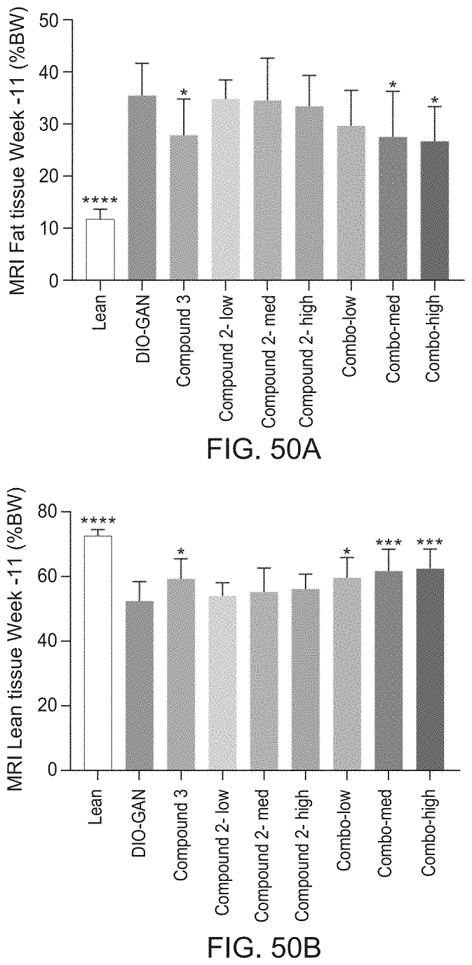

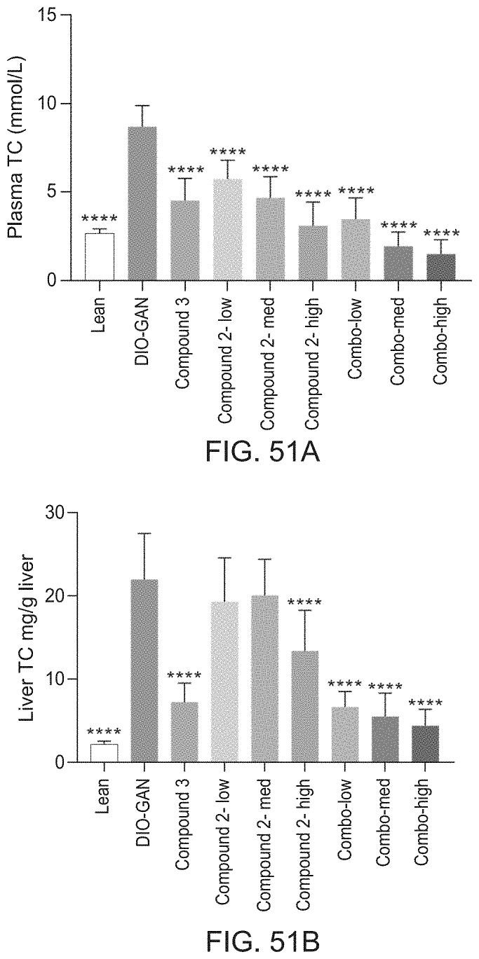

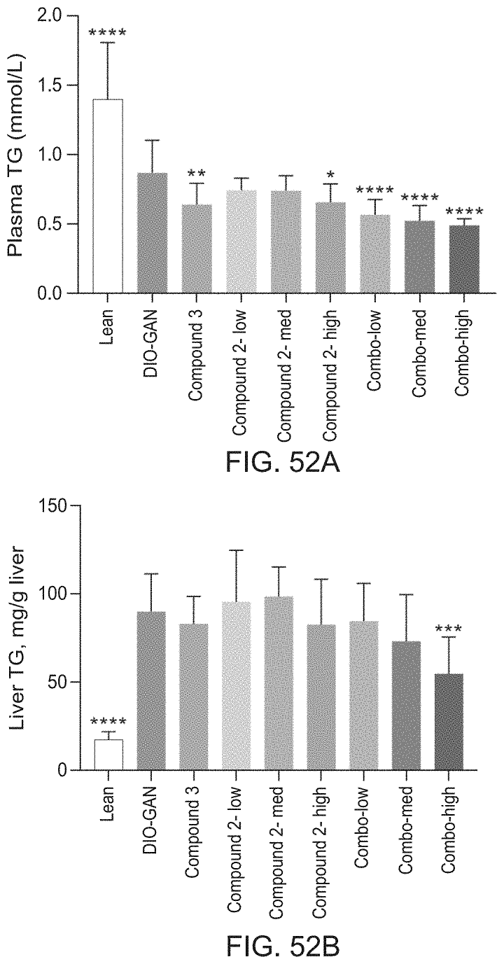

All treatment groups significantly reduced plasma total cholesterol (TC, FIG. 51A), with the greatest reductions seen with combination treatment (Combo-med and Combo-high); similar trends were observed for TC in the liver (FIG. 51B). Reductions in plasma low-density and high-density lipoprotein cholesterols (HDL-c and LDL-c, respectively) were consistent with the effects seen on TC. Compound 3 and Compound 2-high as well as the combination treatments significantly reduced plasma triglycerides (TG, FIG. 52A); liver TG levels were only significantly reduced in the Combo-high group (FIG. 52B).

Effects of Treatment on Live Enzymes

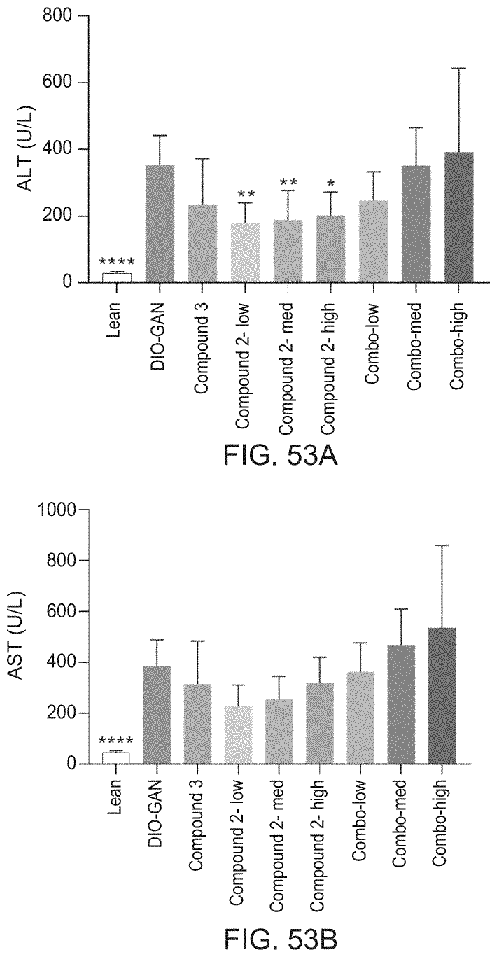

Single agent treatment with Compound 2 (low, med, and high) significantly lowered ALT levels relative to DIO-GAN vehicle control (FIG. 53A); ALT levels were not significantly reduced by combination treatment. AST levels showed a similar trend to ALT although none of the treatment groups were significantly different from the DIO-GAN vehicle control (FIG. 53B). ALP levels were not significantly different from the DIO-GAN vehicle control in any of the treatment groups, although ALP was numerically lower in single agent treatment arms (Compound 3, Compound 2-low and Compound 2-med) and higher in the Combo-med and Combo-high treatments groups relative to DIO-GAN vehicle control (FIG. 54 Error! Reference source not found.).

Liver Histology

NAFLD Activity Score (NAS)

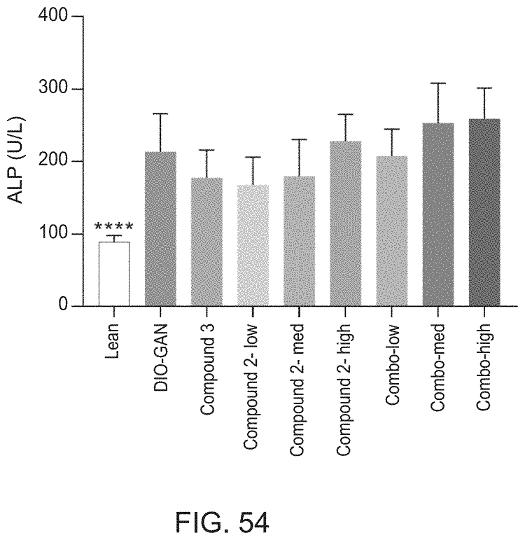

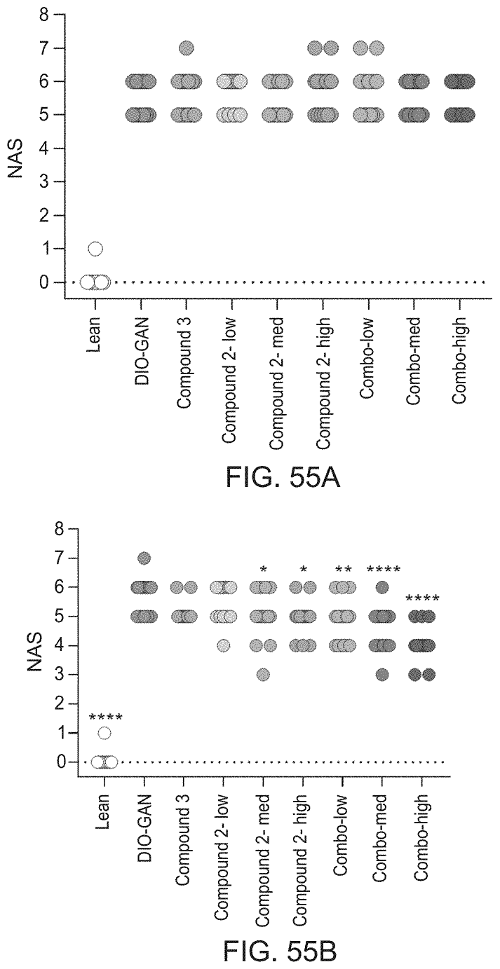

The NAFLD Activity Score (NAS) was used to assess the histological effects of treatment. NAS is defined as the unweighted sum of steatosis, inflammation, and ballooning histological scores and can range from 0-8. NAS was determined for each animal before (baseline) and after 12-weeks of treatment at the end of study. NAS was well balanced across treatment groups with an NAS range of 5-6 at baseline in most mice (FIG. 55A). After 12-weeks of treatment, NAS was significantly improved in most treatment groups with the most profound improvements seen with combination treatment (FIG. 55B). In the Compound 3 treatment group, 56% of the animals showed a ≥1-pt NAS improvement, compared to 31%, 27%, and 62% in the Compound 2-low, Compound 2-med, and Compound 2-high treatment groups, respectively (Table 28). Combination treatment was more effective, with 69%, 81%, and 100% of mice showing ≥1-pt NAS improvement in the Combo-low, Combo-med, and Combo-high combination treatment groups, respectively. In addition, the magnitude of NAS improvement was greater in the combination treatment groups. While 0% of mice in the Compound 3 treatment group improved NAS by ≥1-pt, 19%, 25%, and 43% of mice in the Combo-low, Combo-med, and Combo-high combination arms, respectively, achieved ≥2-pt NAS improvement. These results were superior to the Compound 2 single agent treatment arms, in which 0%, 7%, and 25% of mice achieved ≥2-pt NAS improvement in the Compound 2-low, Compound 2-med, and Compound 2-high dose groups, respectively.

TABLE 28
Effects of Treatment on NAFLD Activity Score (NAS)

| | | | Worsening | | | | | | | |
|---|---|---|---|---|---|---|---|---|---|---|
| | No change | | % (n) | | Improving % (n) | | | | | |
| Treatment (n) | % (n) | | ≥1-pt | | 1-pt | | ≥2-pt | | Total | |
| Lean Control (10) | 80% | (8) | 10% | (1) | 10% | (1) | 0 | | 10% | (1) |
| DIO-GAN Control (16) | 68.75% | (11) | 25% | (4) | 6.25% | (1) | 0 | | 6.25% | (1) |
| Compound 1 (16) | 43.7% | (7) | 0 | | 56.25% | (9) | 0 | | 56.25% | (9) |
| Compound 2-low (16) | 56.25% | (9) | 12.5% | (2) | 31.25% | (5) | 0 | | 31.25% | (5) |
| Compound 2-med (15) | 73.33% | (11) | 0 | | 20% | (3) | 6.7% | (1) | 26.7% | (4) |
| Compound 2-high (16) | 31.25% | (5) | 6.25% | (1) | 37.5% | (6) | 25% | (4) | 62.5% | (10) |
| Combo-low (16) | 18.75% | (3) | 12.5% | (2) | 50% | (8) | 18.75% | (3) | 68.75% | (11) |

| | | | | | | | | | |
|---|---|---|---|---|---|---|---|---|---|
| Combo-med (16) | 18.75% | (3) | 0 | 56.25% | (9) | 25% | (4) | 81.25% | (13) |

| | | | | | | | | |
|---|---|---|---|---|---|---|---|---|
| Combo-high (14) | 0 | 0 | 57.1% | (8) | 42.9% | (6) | 100% | (14) |

Steatosis

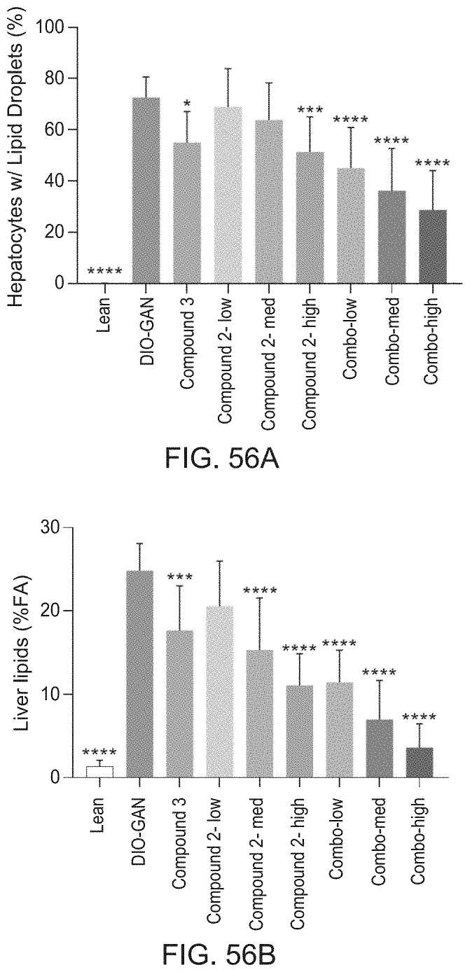

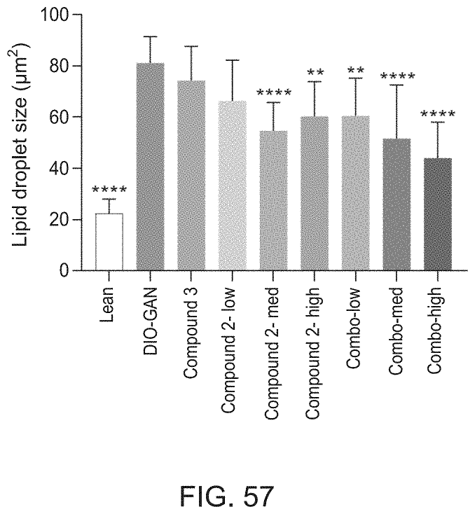

NAS improvements were largely driven by greater reductions in steatosis (Table 29). In the Compound 3 group, 81% showed improved steatosis at the end of treatment, although the maximum improvement was 1-pt. In the Compound 2 single agent treatment groups, dose-dependent increases in the percentage of mice showing steatosis improvement were seen, corresponding to 31%, 47%, and 81% of mice in the Compound 2-low, Compound 2-med, and Compound 2-high dose groups, respectively. In each Compound 2 single agent treatment group one mouse (i.e., ˜6%) showed a ≥2-pt improvement in steatosis. In contrast, 25%, 31%, and 71% of mice in the Combo-low, Combo-med, and Combo-high treatment groups showed ≥2-pt steatosis improvement, respectively. These effects were supported by quantitative liver histomorphometry, which showed a reduced percentage of hepatocytes containing lipid droplets (FIG. 56A) and lower levels of liver lipids (FIG. 56B), as well as smaller lipid droplet size (FIG. 57).

TABLE 29
Effects of Treatment on Histological Steatosis Score

| | No change | | Improving % (n) | | | | | |
|---|---|---|---|---|---|---|---|---|
| Treatment (n) | % (n) | | 1-pt | | >2-pt | | Total | |
| Lean Control (10) | 100% | (10) | 0 | | 0 | | 0 | |
| DIO-GAN Control (16) | 81.25% | (13) | 18.75% | (3) | 0 | | 18.75% | (3) |
| Compound 3 (16) | 18.75% | (3) | 81.25% | (13) | 0 | | 81.25% | (13) |
| Compound 2 Low (16) | 68.75% | (11) | 25% | (4) | 6.25% | (1) | 31.25% | (5) |
| Compound 2 Med (15) | 53.33% | (8) | 40% | (6) | 6.7% | (1) | 46.7% | (7) |
| Compound 2 High (16) | 18.75% | (3) | 75% | (12) | 6.25% | (1) | 81.25% | (13) |
| Combo Low (16) | 6.25% | (1) | 68.75% | (11) | 25% | (4) | 93.75% | (15) |
| Combo Med (16) | 6.25% | (1) | 62.5% | (10) | 31.25% | (5) | 93.75% | (15) |

| | | | | | | | |
|---|---|---|---|---|---|---|---|
| Combo High (14) | 0 | 28.6% | (4) | 72.4% | (10) | 100% | (14) |

The combination of Compound 3 and Compound 2 resulted in greater improvements in liver steatosis. Liver steatosis was determined by histology at baseline and end of treatment for each individual mouse. Table 29 shows the percentage of mice within each treatment group with no change or improving (1-pt and ≥2-pt decrease from baseline) steatosis score. Total represents the percentage of mice in each treatment group showing at least 1-pt steatosis improvement from baseline.

Ballooning, Inflammation, and Fibrosis

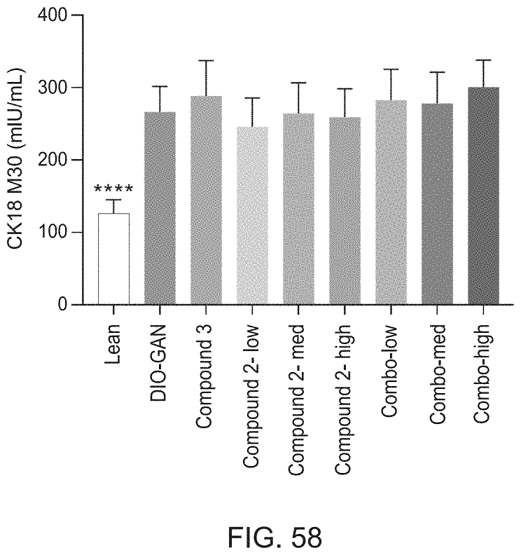

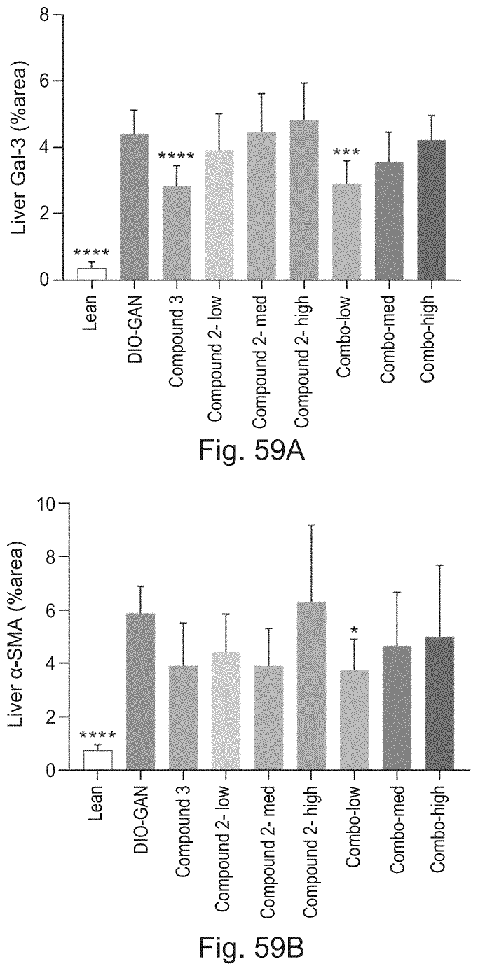

Hepatocellular ballooning, an indicator of apoptosis, was infrequently observed and not significantly changed by any of the treatments. CK18 M30, a plasma biomarker associated with apoptosis, was also not significantly different between treatment groups and the DIO-GAN vehicle control (FIG. 58). Lobular inflammation was not significantly improved by treatment, although improvements in inflammation scores were observed, albeit infrequently, in the Compound 2-low, Compound 2-med, and Compound 2-high treatment groups as well as in the Combo-med combination group (Error! Reference source not found). To further assess inflammation, protein expression Galectin-3 (Gal-3), a marker of inflammatory lymphocyte infiltration, was determined by immunohistochemistry (IHC) staining of the liver. Treatment with Compound 3 alone and in combination with Compound 2 (Combo-low) resulted in lower levels of Gal-3 expression in the liver relative to DIO-GAN vehicle control (FIG. 59A).

TABLE 30
Effects of Treatment on Lobular Inflammation

| | Worsening | | No change | | Improving | |
|---|---|---|---|---|---|---|
| Treatment (n) | % (n) | | % (n) | | % (n) | |
| Lean Control (10) | 10% | (1) | 80% | (8) | 10% | (1) |
| DIO-GAN Control (16) | 31.25% | (5) | 68.75% | (11) | 0 | |
| Compound 3 (16) | 31.25% | (5) | 68.75% | (11) | 0 | |
| Compound 2 Low (16) | 25% | (4) | 68.75% | (11) | 6.25% | (1) |
| Compound 2 Med (15) | 20% | (3) | 73.3% | (11) | 6.7% | (1) |
| Compound 2 High (16) | 31.25% | (5) | 56.25% | (9) | 12.5% | (2) |
| Combo Low (16) | 31.25% | (5) | 68.75% | (11) | 0 | |
| Combo Med (16) | 31.25% | (5) | 62.5% | (10) | 6.25% | (1) |

| | | | | | |
|---|---|---|---|---|---|
| Combo High (14) | 35.7% | (5) | 64.3% | (9) | 0 |

Inflammation was not significantly improved by treatment. Lobular inflammation was determined by histology at baseline and end of treatment. Table 30 shows the percentage of mice within each treatment group with worsening (≥1-pt increase from baseline), no change, or improving (≥1-pt decrease from baseline) lobular inflammation scores.

Improvements in fibrosis stage were more frequently observed in the combination agent treatment groups compared with the single agent arms, although differences did not reach significance (Table 31). Changes in Col1a1 protein expression, as determined by IHC staining of the liver, were not significantly different between treatment groups. Numeric reductions in α-SMA, a marker of hepatic stellate cell activation, were observed in the Compound 3, Compound 2-low, and Compound 2-med treatment group. Significant reductions were only observed in the Combo-low treatment group (FIG. 59B).

TABLE 31
Effects of Treatment on Liver Fibrosis

| | Worsening | | No change | | Improving | |
|---|---|---|---|---|---|---|
| Treatment (n) | % (n) | | % (n) | | % (n) | |
| Lean Control (10) | 0 | | 100% | (10) | 0 | |
| DIO-GAN Control (16) | 31.25% | (5) | 68.75% | (11) | 0 | |
| Compound 3 (16) | 37.5% | (6) | 56.25% | (9) | 6.25% | (1) |
| Compound 2 Low (16) | 31.25% | (5) | 62.5% | (10) | 6.25% | (1) |
| Compound 2 Med (15) | 26.7% | (4) | 60% | (9) | 13.33% | (2) |
| Compound 2 High (16) | 25% | (4) | 68.75% | (11) | 6.25% | (1) |
| Combo Low (16) | 37.5% | (6) | 43.75% | (7) | 18.75 | (3) |
| Combo Med (16) | 18.75% | (3) | 75% | (12) | 6.25% | (1) |
| Combo High (14) | 21.4% | (3) | 50% | (7) | 28.6% | (4) |

Fibrosis improvement more frequently observed with combination treatment. Liver fibrosis stage was determined by histology at baseline and end of treatment. Table 31 shows the percentage of mice within each treatment group with worsening (≥1-stage increase from baseline), no change, or improving (≥1-stage decrease from baseline) fibrosis.

Liver Transcriptomics Analysis by RNAseq

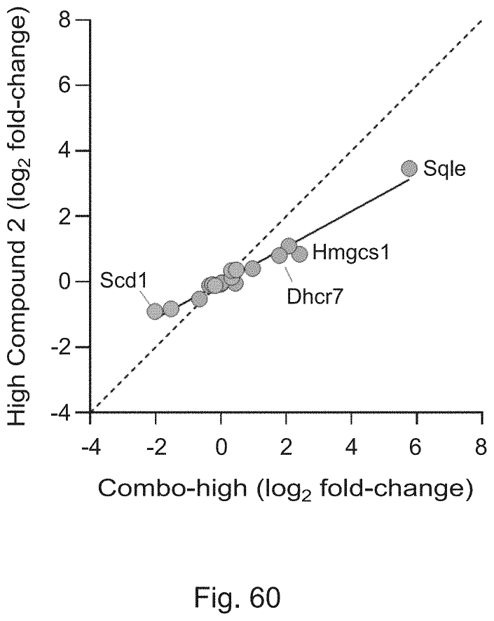

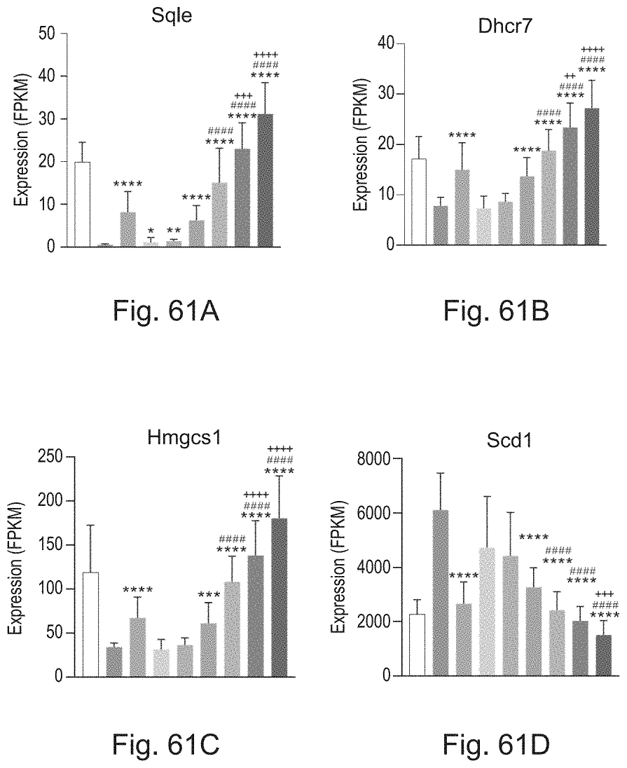

Terminal liver samples (n=10) from treatment groups were processed for transcriptomics analysis by RNAseq. Differentially expressed genes (DEGs) were identified compared to DIO-GAN vehicle control. DEGs were identified in all treatment groups; fewest in Compound 2-low (987) and the largest number of DEGs in Combo-high (3533) treatment group. Comparison of Compound 2-high to Combo-high indicated that genes involved in energy and lipid metabolism were differentially expressed to a greater extent by combination treatment (FIG. 60). This is indicated by comparison of the slopes for the line of identity (dotted line, slope=1) to the linear regression line (solid line, slope=0.55) of fold-change values. Squalene epoxidase (Sqle) and 7-dehydrocholesterol reductase (Dhcr7), enzymes involved in cholesterol metabolism were expressed at significantly higher levels in the combination groups relative to single agent treatment (FIGS. 61A and 61B). Hydroxymethylglutaryl-CoA synthase (Hmgcs1), a key enzyme in energy metabolism, showed a similar pattern of expression (FIG. 61C). Stearoyl-CoA desaturase (Scd1), an enzyme involved in fatty acid metabolism, was reduced by Compound 3 treatment, and further reduced by combination treatment (FIG. 61D).

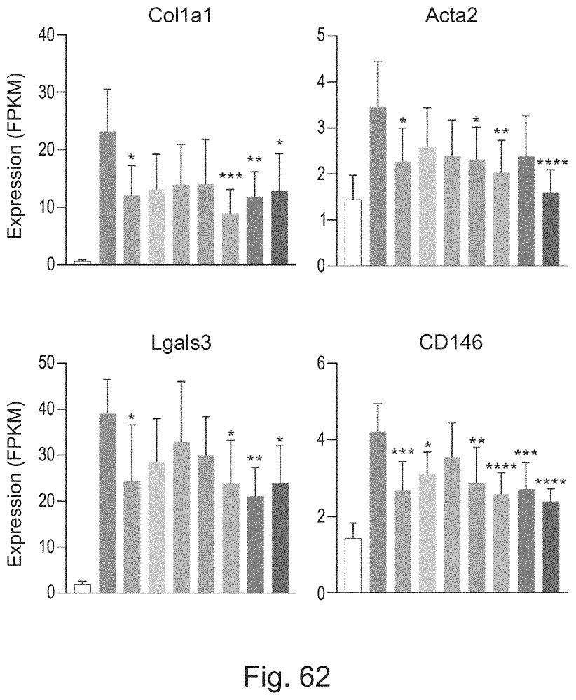

Lastly, we examined the expression of select genes associated with fibrosis and inflammation including collagen type I alpha 1 (Col1a1), actin alpha 2 smooth actin (Acta2), Galectin 3 (Lgals3), and melanoma cell adhesion molecule (CD146). In general, Compound 3 alone and in combination with Compound 2 significantly reduced expression of these genes relative to DIO-GAN vehicle control (FIG. 62); combination treatment arms were not significantly different from Compound 3 treatment alone.

### SUMMARY

The mouse diet-induced obese Gubra-Amylin NASH (DIO-GAN) model was used to evaluate the efficacy of Compound 3 and Compound 2 as single agents and in combination on metabolic and histopathological parameters of NASH and fibrosis. This model has been extensively characterized and recapitulates many aspects of human NASH (Hansen 2020) without the use of hepatotoxic agents to induce disease. In this model, mice were maintained on a diet high in fat, cholesterol, and fructose (GAN diet) for >35 weeks. Prior to therapeutic intervention, mice were biopsied to assess NAFLD activity score (NAS) and fibrosis severity by histology; only mice with a baseline steatosis score of >2 and fibrosis stage >1 were used in the study. Importantly, this preselection step ensures that only mice with significant NAFLD activity were used in the study. In addition, knowledge of the baseline NAS allows for therapeutic responses to be evaluated not only relative to the DIO-GAN vehicle control but also relative to individual baseline values. DIO-GAN mice were treated with Compound 3 and Compound 2 alone and in combination for 12 weeks and maintained on the GAN diet throughout the study. Mice were treated with a single dose level of Compound 3, alone or in combination with 3 dose levels (low [0.3 mg/kg], med [2 mg/kg], and high [10 mg/kg]) of Compound 2 in order to maximize the ability to discern potential additive therapeutic effects.

The combination of Compound 3 and Compound 2 showed greater reductions in NAS relative to single agent treatments both in terms of the percentage of mice showing reduction in NAS and the magnitude of NAS improvement. Improvements in NAS were largely driven by greater reductions in steatosis, which was associated with larger reductions in plasma and liver total cholesterol and triglycerides. The greater overall effects seen with the combination treatment did not appear to be driven by higher exposure of the individual drugs in the combination treatment groups (Table 32). In addition, although changes in body weight may have contributed to NAS improvements, body weight reductions were similar between Compound 3 and combination treatments groups (Combo-low and Combo-med), suggesting that weight loss alone does not fully explain the greater anti-steatotic activity of the combination treatment. Instead, combination treatment had greater effects on the expression of genes related to energy and lipid metabolism. These results suggest that the combination of Compound 3 and Compound 2 appear to have at least an additive effect on these pathways and likely responsible for the greater anti-steatotic activity observed.

Histological improvement in inflammation and fibrosis were not significantly improved by treatment. Evidence of fibrosis improvement was noted, however, including a larger number of mice showing fibrosis improvement with the combination. In addition, transcriptomics analysis identified key markers of fibrosis and inflammation that were reduced by combination treatment. Expression of these genes was also reduced by Compound 3 treatment, suggesting that FXR agonism may be the main driver for effect on fibrosis and inflammation at the gene expression level. In this case, FXR agonism can be considered complementary to the more antisteatotic mechanism of THR-β agonism. Together with the greater antisteatotic effects observed by combining Compound 3 and Compound 2, these results suggest that this combination could address multiple aspects of NASH disease.

### REFERENCES

Kleiner D E, Brunt E M, Van Natta M, Behling C, Contos M J, Cummings O W, Ferrell L D, Liu Y C, Torbenson M S, Unalp-Arida A, Yeh M, McCullough A J, Sanyal A J; Nonalcoholic Steatohepatitis Clinical Research Network. Design and validation of a histological scoring system for nonalcoholic fatty liver disease. Hepatology. 2005 June; 41(6):1313-21.

Hansen, H. H., Ægidius, H. M., Oró, D. et al. Human translatability of the GAN diet-induced obese mouse model of non-alcoholic steatohepatitis. BMC Gastroenterol 20, 210 (2020).

Supplemental

TABLE 32
Trough plasma drug concentrations determined by LC-
MS/MS Mean trough analyte concentration (SD), ng/mL

| | | Compound 3 | |
|---|---|---|---|
| | | glucuronide | |
| Treatment Group (n) | Compound 3 | metabolite | Compound 2 |
| Compound 3 (16) | 7.4 (3.8) | 52.7 (26.0) | ND |
| Compound 2-med (15) | ND | ND | 135.5 (62.0) |
| Compound 2-high (16) | ND | ND | 320.0 (189.9) |
| Combo-med (16) | 5.8 (3.5) | 53.1 (36.8) | 49.5 (42.9) |
| Combo-high (14) | 3.3 (1.8) | 49.0 (23.7) | 143.0 (87.9) |

Terminal plasma samples collected by cardiac puncture 21-24 hours post final treatment dose (i.e., trough) were analyzed by LC-MS/MS. Glucuronide metabolite of Compound 3. Values represent mean and standard deviation (SD). ND, not determined.

### Example 20

A Multi-Center, Randomized, Double-Blind, Dose-Ranging, Placebo-Controlled, Clinical Study to Evaluate the Safety, Pharmacokinetics, Pharmacodynamics, and Preliminary Efficacy of Orally Administered Compound 1 in Patients with Presumed Non-Cirrhotic Non-Alcoholic Steatohepatitis (NASH) is performed. The total duration of study participation is approximately 22 weeks, consisting of a 6-week Screening Period, a 12-week Treatment Period and a 4-week Follow-up Period.

Approximately 80 clinically or histologically diagnosed adult non-cirrhotic presumed NASH patients who meet study eligibility criteria are enrolled and randomized at an overall ratio of 2:1 into 3 dose groups and placebo across 2 parts of the study. Eligibility criteria include: overweight or obese, with NASH by clinical diagnosis or biopsy, ALT≥43 U/L (men) or ≥28 U/L (women), MRI-cT1 liver inflammation >800 ms.

Part 1 of this study assesses 10 mg of Compound 1, the highest dose studied in Example 2. In Example 2, this dose led to >90% suppression of plasma VAP-1/SSAO-specific amine oxidase activity in healthy participants. NASH patients are expected to have a higher baseline level of VAP-1, and thus the PD effect of Compound 1 in NASH patients may differ from healthy participants. A higher dose of up to 20 mg may be enrolled based on assessment of safety and PK with the 10 mg dose, and a lower dose of 4 mg may be enrolled based on observation of a robust PD effect of Compound 1 on plasma VAP-1/SSAO activity in the 10 mg cohort, thus minimizing the number of patients exposed until the PK and PD effects of Compound 1 can be confirmed in Part 1 of the study.

In Part 1, approximately 30 patients will receive 10 mg Compound 1 (n=20) or matching placebo (n=10) orally once daily, for 12 weeks. Approximately 12 randomized patients take part in an intensive PK and PD collection after the first dose (Week 0/Day 1), at Week 6, and after the last dose of study drug (Week 12). Randomization will ensure approximately 8 patients in the Compound 1 group and approximately 4 patients in the placebo group are assigned to the PK/PD sub-study. Patients who are not participating in the PK/PD sub-study will have trough PK/PD sampling only.

An interim analysis is performed once all patients in Part 1 have completed Week 6 assessments. Interim PK and PD data are assessed. Blinded safety data is also reviewed. If robust VAP-1/SSAO activity suppression is observed and available PK from Part 1 suggests that a lower dose may also possibly lead to robust VAP-1/SSAO activity suppression, enrollment in Part 2 with 4 mg Compound 1 may be initiated. If safety data indicates 10 mg Compound 1 is overall safe and well-tolerated, and available PK from Part 1 predicts that targeted exposures for a dose up to 20 mg will be below an upper limit of 5400 ng·hr/mL for AUC_{0-24 hr} and 768 ng/mL for Cₘₐₓ, enrollment in Part 2 with a dose up to 20 mg Compound 1 may be initiated.

In Part 2, approximately 50 patients receive 4 mg Compound 1 (n=20), and/or up to 20 mg Compound 1 (n=20), or matching placebo (n=10) orally once daily, for 12 weeks. Approximately 15 randomized patients take part in an intensive PK and PD collection after the first dose (Week 0/Day 1), at Week 6, and after the last dose of study drug (Week 12). Randomization will ensure approximately 6 patients at each Compound 1 dose level and approximately 3 patients in the placebo group are assigned to the PK/PD sub-study. Patients who are not participating in the PK/PD sub-study will have trough PK/PD sampling only.

### PK/PD Sub-Study and Trough PK/PD Sampling

Plasma samples are collected for measurement of plasma concentrations of study drug and metabolites. Urine samples are collected for measurement of urine concentrations of study drug and metabolites. Patients who are not participating in the PK/PD sub-study undergo trough PK sampling only. Blood collection for NASH/fibrosis markers (CK-18 (M30 and M65), PIIINP, TIMP-1, HA, PRO-C₃, and C₃M) and inflammation markers (hs-CRP, IL-6, ICAM-1, and VCAM-1) are also conducted. The following exploratory fibrosis scores may also be calculated: FIB-4, enhanced liver fibrosis (ELF), and NAFLD. The PRO-C₃/C₃M ratio may be calculated. Patients are monitored for adverse events.

Plasma and urine samples are collected pre-dose for all patients on days 1, 2, 15, 29, 43, 57, and 85. For patients within the PK/PD sub-study: On days 1 (week 0), 43 (week 6), and 85 (week 12), plasma samples are collected at 30 minutes, 1 h, 2 h, 4 h, 6 h, and 8 h post-dose, and total urine collection is performed from 0-8 h post-dose. On days 2 (week 0), 44 (week 6), and 96 (week 12), samples are collected (24 h post-dose). On days 87 and 88, samples are collected (48 and 72 hours post-week 12 dose). Markers including ALT, AST, ALP, and total bilirubin will be monitored.

1. A method of treating non-alcoholic steatohepatitis (NASH) in a patient in need thereof, comprising administering to the patient an SSAO inhibitor and a THR-β agonist, wherein the SSAO inhibitor is a compound of formula (1): 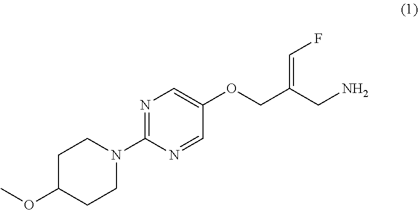 [SMILES: COC1CCN(c2ncc(OC/C(=C/F)CN)cn2)CC1]
or a pharmaceutically acceptable salt thereof.

2. The method of claim 1, wherein the SSAO inhibitor is a tosylate salt of the compound of formula (1).

3. The method of claim 1, wherein the THR-β agonist is a compound of formula (II) 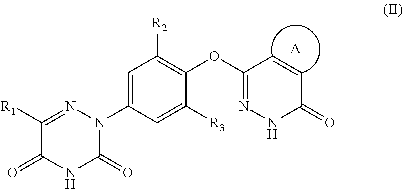
wherein:
R₁ is selected from the group consisting of hydrogen, cyano, substituted or unsubstituted C₁₋₆ alkyl, and substituted or unsubstituted C₃₋₆ cycloalkyl, the substituent being selected from the group consisting of halogen atoms, hydroxy, and C₁₋₆ alkoxy;
R₂ and R₃ are each independently selected from the group consisting of halogen atoms and substituted or unsubstituted C₁₋₆ alkyl, the substituent being selected from the group consisting of halogen atoms, hydroxy, and C₁₋₆ alkoxy;
ring A is a substituted or unsubstituted saturated or unsaturated C₅₋₁₀ aliphatic ring, or a substituted or unsubstituted C₅₋₁₀ aromatic ring, the substituent being one or more substances selected from the group consisting of hydrogen, halogen atoms, hydroxy, —OCF₃, —NH₂, —NHC₁₋₄ alkyl, —N(C₁₋₄ alkyl)₂, —CONH₂, —CONHC₁₋₄ alkyl, —CON(C₁₋₄ alkyl)₂, —NHCOC₁₋₄ alkyl, C₁₋₆ alkyl, C₁₋₆ alkoxy and C₃₋₆ cycloalkyl, and when two substituents are contained, the two substituents can form a ring structure together with the carbon connected thereto; and
the halogen atoms are selected from the group consisting of F, Cl and Br,
or a pharmaceutically acceptable salt thereof.

4. The method of claim 1, wherein the THR-β agonist is a compound of formula (IIa) 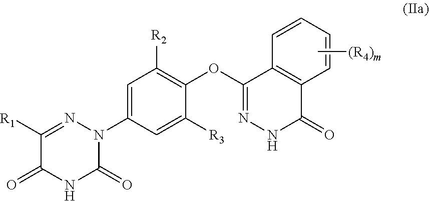
wherein:
R₄ is selected from the group consisting of hydrogen, halogen atoms, hydroxy, —OCF₃, —NH₂, —NHC₁₋₄ alkyl, —N(C₁₋₄ alkyl)₂, —CONH₂, —CONHC₁₋₄ alkyl, —CON(C₁₋₄ alkyl)₂, —NHCOC₁₋₄ alkyl, C₁₋₆ alkyl, C₁₋₆ alkoxy and C₃₋₆ cycloalkyl;
m is an integer from the range 1 to 4; and
the halogen atoms are selected from the group consisting of F, Cl and Br.
or a pharmaceutically acceptable salt thereof.

5. The method of claim 3, wherein R₄ is selected from the group consisting of hydrogen, halogen atoms, hydroxy, —OCF₃, C₁₋₆ alkyl, C₁₋₆ alkoxy and C₃₋₆ cycloalkyl; and
m is an integer from the range 1 to 3.

6. The method of claim 3, wherein R₁ is selected from the group consisting of hydrogen, cyano, and substituted or unsubstituted C₁₋₆ alkyl, the substituent being selected from the group consisting of halogen atoms, hydroxy, and C₁₋₆ alkoxy; and
the halogen atoms are selected from the group consisting of F, Cl and Br.

7. The method of claim 1, wherein the THR-β agonist is a compound of formula (2): 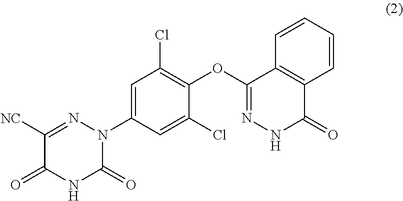 [SMILES: N#Cc1nn(-c2cc(Cl)c(Oc3n[nH]c(=O)c4ccccc34)c(Cl)c2)c(=O)[nH]c1=O]
or a pharmaceutically acceptable salt thereof.

8. The method of claim 7, wherein the THR-β agonist is a potassium salt of the compound of formula (2).

9. The method of claim 1, wherein the SSAO inhibitor and the THR-β agonist are administered:
(i) simultaneously; or
(ii) sequentially.

10. (canceled)

11. The method of claim 1, wherein the patient has liver fibrosis.

12. The method of claim 1, wherein the patient also has:
(i) diabetes mellitus; and/or
(ii) a cardiovascular disorder.

13. (canceled)

14. The method of claim 1, wherein the treatment period is the remaining lifespan of the patient.

15. The method of claim 1, wherein the SSAO inhibitor is administered once daily.

16. The method of claim 15, wherein the SSAO inhibitor is administered to the patient at a dose of from about 1 mg to about 40 mg daily.

17. The method of claim 1, wherein the THR-β agonist is administered once daily.

18. The method of claim 17, wherein the THR-β agonist is administered at a dose from about 0.5 mg to about 90 mg daily, wherein the THR-β agonist is the compound of formula (2) or a pharmaceutically acceptable salt thereof.

19. The method of claim 1, wherein the SSAO inhibitor is administered at a dose from about 1 mg to about 40 mg daily and the THR-β agonist is administered at a dose from about 0.5 mg to about 90 mg daily, wherein the SSAO inhibitor is a tosylate salt of the compound of formula (1) and the THR-β agonist is the compound of formula (2) or a pharmaceutically acceptable salt thereof.

20. The method of claim 1, wherein the administration comprises:
(i) administering the SSAO inhibitor daily for a treatment period of one or more weeks; and/or
(ii) administering the THR-β agonist daily for a treatment period of one or more weeks.

21. (canceled)

22. The method of claim 1, wherein the administration reduces at least one of steatosis, liver inflammation, or liver fibrosis compared to administration with a monotherapy of the SSAO inhibitor or the THR-β agonist.

23. The method of claim 22, wherein the administration:
(i) reduces steatosis compared to administration with a monotherapy of the SSAO inhibitor or the THR-β agonist;
(ii) reduces liver inflammation compared to administration with a monotherapy of the SSAO inhibitor or the THR-β agonist; and/or
(iii) reduces liver fibrosis compared to administration with a monotherapy of the SSAO inhibitor or the THR-β agonist.

24-25. (canceled)

26. A method of reducing hepatic inflammation in a patient in need thereof, comprising administering to the patient a therapeutically effective amount of an SSAO inhibitor and a therapeutically effective amount of a THR-β agonist, wherein the SSAO inhibitor is a compound of formula (1): 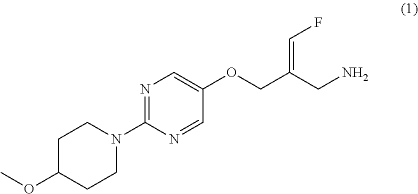 [SMILES: COC1CCN(c2ncc(OC/C(=C/F)CN)cn2)CC1]
or a pharmaceutically acceptable salt thereof, and the THR-β agonist is a compound of formula (2): 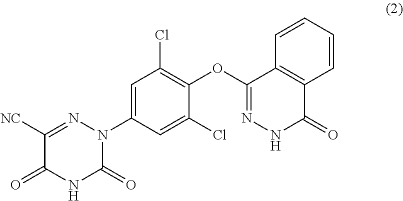 [SMILES: N#Cc1nn(-c2cc(Cl)c(Oc3n[nH]c(=O)c4ccccc34)c(Cl)c2)c(=O)[nH]c1=O]
or a pharmaceutically acceptable salt thereof.

27. The method of claim 26, wherein the SSAO inhibitor is a tosylate salt of the compound of formula (1) and the THR-β agonist is a potassium salt of the compound of formula (2).

28. The method of claim 26, wherein the compound of formula (1), or a pharmaceutically salt thereof, and the compound of formula (2), or a pharmaceutically salt thereof, are each administered once daily to the patient.

29. The method of claim 26, wherein the method reduces hepatic inflammation without increasing LDL-c levels in the patient.

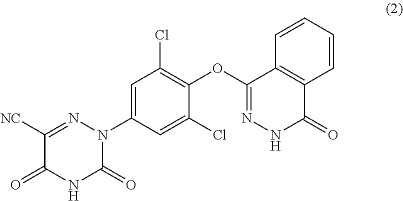 [SMILES: N#Cc1nn(-c2cc(Cl)c(Oc3n[nH]c(=O)c4ccccc34)c(Cl)c2)c(=O)[nH]c1=O]

30. The method of claim 29, wherein the method:
(i) also reduces hepatic steatosis; and/or
(ii) decreases LDL-c levels in the patient.

31-32. (canceled)

33. The method of claim 29, wherein the compound of formula (1), or a pharmaceutically salt thereof, and the compound of formula (2), or a pharmaceutically salt thereof, are each administered once daily to the patient.

34. A fixed-dose pharmaceutical composition for oral administration, comprising a compound of formula (1): 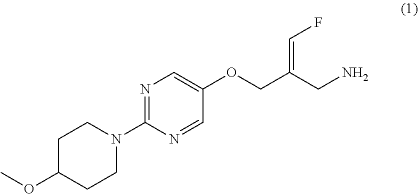 [SMILES: COC1CCN(c2ncc(OC/C(=C/F)CN)cn2)CC1]
or a pharmaceutically acceptable salt thereof, and a compound of formula (2): 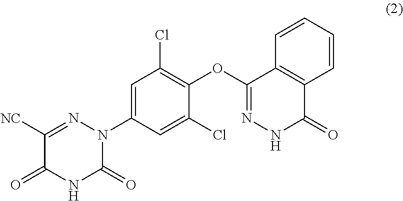 [SMILES: N#Cc1nn(-c2cc(Cl)c(Oc3n[nH]c(=O)c4ccccc34)c(Cl)c2)c(=O)[nH]c1=O]
or a pharmaceutically acceptable salt thereof.

35. The fixed-dose pharmaceutical combination of claim 34, wherein the SSAO inhibitor is a tosylate salt of the compound of formula (1) and the THR-β agonist is a potassium salt of the compound of formula (2).

36. The fixed-dose pharmaceutical composition of claim 33, wherein the composition comprises:
(i) from about 1 mg to about 40 mg of the compound of formula (1), or a pharmaceutically salt thereof, and from about 0.5 mg to about 30 mg of the compound of formula (2), or a pharmaceutically acceptable salt thereof; or
(ii) from about 1 mg to about 40 mg of the compound of formula (1), or a pharmaceutically salt thereof, and from about 5 mg to about 20 mg of the compound of formula (2), or a pharmaceutically acceptable salt thereof.

37. (canceled)
